SEQUENCE LISTING (78 sequences)

SEQ ID NO: 1 - DNA, 1452 bp, Bifidobacterium longum subsp. infantis
tggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgg

SEQ ID NO: 2 - DNA, 1537 bp, Bifidobacterium longum subsp. infantis
tttttgtggagggttcgattctggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctttctacggaga

SEQ ID NO: 3 - DNA, 472 bp, Bifidobacterium longum subsp. infantis
cgctggcggcgtgcttacacatgcaagtcgaacgggatccatcgggctttgcttggtggtgagagtggcgaacgggtgaataatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtagtgtacccgttgaataagcaccggttaactcgtgccagcagccgcgg

SEQ ID NO: 4 - DNA, 1458 bp, Bifidobacterium longum subsp. infantis
  misc_feature (50)..(50): n is a, c, g, or t
  misc_feature (176)..(176): n is a, c, g, or t
  misc_feature (234)..(234): n is a, c, g, or t
  misc_feature (266)..(266): n is a, c, g, or t
  misc_feature (353)..(353): n is a, c, g, or t
  misc_feature (363)..(363): n is a, c, g, or t
  misc_feature (370)..(370): n is a, c, g, or t
  misc_feature (398)..(398): n is a, c, g, or t
  misc_feature (437)..(437): n is a, c, g, or t
  misc_feature (451)..(451): n is a, c, g, or t
  misc_feature (453)..(453): n is a, c, g, or t
  misc_feature (462)..(462): n is a, c, g, or t
  misc_feature (467)..(467): n is a, c, g, or t
  misc_feature (474)..(474): n is a, c, g, or t
  misc_feature (522)..(522): n is a, c, g, or t
  misc_feature (528)..(528): n is a, c, g, or t
  misc_feature (548)..(548): n is a, c, g, or t
  misc_feature (721)..(721): n is a, c, g, or t
  misc_feature (807)..(807): n is a, c, g, or t
  misc_feature (809)..(809): n is a, c, g, or t
  misc_feature (813)..(817): n is a, c, g, or t
  misc_feature (827)..(828): n is a, c, g, or t
  misc_feature (857)..(857): n is a, c, g, or t
  misc_feature (862)..(862): n is a, c, g, or t
  misc_feature (868)..(950): n is a, c, g, or t
  misc_feature (997)..(1009): n is a, c, g, or t
  misc_feature (1013)..(1014): n is a, c, g, or t
  misc_feature (1018)..(1019): n is a, c, g, or t
  misc_feature (1028)..(1028): n is a, c, g, or t
  misc_feature (1032)..(1032): n is a, c, g, or t
  misc_feature (1046)..(1047): n is a, c, g, or t
  misc_feature (1051)..(1051): n is a, c, g, or t
  misc_feature (1054)..(1054): n is a, c, g, or t
  misc_feature (1056)..(1068): n is a, c, g, or t
  misc_feature (1089)..(1089): n is a, c, g, or t
  misc_feature (1097)..(1097): n is a, c, g, or t
  misc_feature (1111)..(1111): n is a, c, g, or t
  misc_feature (1113)..(1114): n is a, c, g, or t
  misc_feature (1121)..(1122): n is a, c, g, or t
  misc_feature (1132)..(1133): n is a, c, g, or t
  misc_feature (1146)..(1146): n is a, c, g, or t
  misc_feature (1187)..(1187): n is a, c, g, or t
  misc_feature (1270)..(1270): n is a, c, g, or t
  misc_feature (1272)..(1272): n is a, c, g, or t
  misc_feature (1276)..(1276): n is a, c, g, or t
  misc_feature (1295)..(1295): n is a, c, g, or t
  misc_feature (1337)..(1337): n is a, c, g, or t
  misc_feature (1340)..(1340): n is a, c, g, or t
  misc_feature (1382)..(1383): n is a, c, g, or t
  misc_feature (1393)..(1393): n is a, c, g, or t
  misc_feature (1406)..(1406): n is a, c, g, or t
  misc_feature (1423)..(1423): n is a, c, g, or t
  misc_feature (1431)..(1431): n is a, c, g, or t
  misc_feature (1439)..(1439): n is a, c, g, or t
tttgtggagggttcgattctggctcaggatgaacgctggcggcgtgcttnacacatgcaagtcgaacgggatccatcgggctttgcttggtggtgagagtggcgaacgggtgagtaatgcgtgaccgacctgccccatacaccggaatagctcctggaaacgggtggtaatgccgnatgttccagttgatcgcatggtcttctgggaaactttcgcggtatgggatggggtcgngtcctatcagcttgacggcggggtaacggccnaccgtggcttcgacgggtagccggcctgagagggcgaccggccacattgggactgagatacggcccagactcctacgggaggcagcngtggggaatnttgcacnatgggcgcaagcctgatgcagcgacgcngcgtgagggatggaggcttcgggttgtaaacctcttttntcggggagcaagcntnagtgagttnacccnttgaatnagcaccggctaactacgtgccagcagccgcggtaatacgtagggtgcnagcgtnatccggaattattgggcgtnaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggcngttactgacgcttaggagctaaagcgttgggagcgaacaggattagataccctggtagtccacgccgtaaacggtggatgctggangngggnnnnnttccacgggnnccgtgtcggacgaacgcgttaagcatccngcctngggagnnnnnnnnnnnnnnnnnnnnnnnnnnnnnnnnnnnnnnnnnnnnnnnnnnnnnnnnnnnnnnnnnnnnnnnnnnnnnnnnnnnaagaaccttacctgggcttgacatgttcccgacgatcccagaggggnnnnnnnnnnnnngggnnggtnnacaggtggngcanggtcgtcgtcagcnngtgncgngnnnnnnnnnnnnnagtcccgcaacgagcgcaacnctcgcccngtgttgccagcggntnntgccggnnactcacgggnnaccgccggggttnactcggaggaaggtggggatgacgtcagatcatcatgcccnttacgtccagggcttcacgcatgctacaatggccggtacaacgggatgcgacgcggcgacgcggagcggatccctgaaaaccngnctcngttcggatcgcagtctgcnactcgactgcgtgaaggcggagtcgctagtaatcgcgaatcngcngcgtcgcggtgaatgcgttcccgggccttgtacacaccgccnntcaagtcatnaaagtgggcagcncccgaagccggtggccnaacccctngtgggatngagccgtctaaggtgaggc

SEQ ID NO: 5 - DNA, 1515 bp, Bifidobacterium longum subsp. infantis
tttgatcatggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctta

SEQ ID NO: 6 - DNA, 658 bp, Bifidobacterium longum subsp. infantis
ccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactg

SEQ ID NO: 7 - DNA, 1447 bp, Bifidobacterium longum subsp. infantis
gatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggact

SEQ ID NO: 8 - DNA, 1530 bp, Bifidobacterium adolescentis
tttgtggagggttcgattctggctcaggatgaacgctggcggcgtgcttaacacatgcaagtcgaacgggatcggctggagcttgctccggccgtgagagtggcgaacgggtgagtaatgcgtgaccgacctgccccatacaccggaatagctcctggaaacgggtggtaatgccggatgctccagttggatgcatgtccttctgggaaagattctatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctccttt

SEQ ID NO: 9 - DNA, 1459 bp, Bifidobacterium animalis subsp. animalis
  misc_feature (29)..(29): n is a, c, g, or t
  misc_feature (61)..(61): n is a, c, g, or t
  misc_feature (64)..(64): n is a, c, g, or t
  misc_feature (69)..(70): n is a, c, g, or t
  misc_feature (75)..(76): n is a, c, g, or t
  misc_feature (82)..(82): n is a, c, g, or t
  misc_feature (85)..(86): n is a, c, g, or t
  misc_feature (108)..(108): n is a, c, g, or t
  misc_feature (155)..(155): n is a, c, g, or t
  misc_feature (167)..(168): n is a, c, g, or t
  misc_feature (175)..(175): n is a, c, g, or t
  misc_feature (178)..(178): n is a, c, g, or t
  misc_feature (191)..(192): n is a, c, g, or t
  misc_feature (222)..(222): n is a, c, g, or t
  misc_feature (277)..(277): n is a, c, g, or t
  misc_feature (305)..(305): n is a, c, g, or t
  misc_feature (325)..(328): n is a, c, g, or t
  misc_feature (346)..(347): n is a, c, g, or t
  misc_feature (419)..(419): n is a, c, g, or t
  misc_feature (426)..(426): n is a, c, g, or t
  misc_feature (434)..(434): n is a, c, g, or t
  misc_feature (440)..(440): n is a, c, g, or t
  misc_feature (459)..(459): n is a, c, g, or t
  misc_feature (514)..(514): n is a, c, g, or t
  misc_feature (528)..(528): n is a, c, g, or t
  misc_feature (545)..(545): n is a, c, g, or t
  misc_feature (554)..(554): n is a, c, g, or t
  misc_feature (726)..(726): n is a, c, g, or t
  misc_feature (728)..(728): n is a, c, g, or t
  misc_feature (746)..(746): n is a, c, g, or t
  misc_feature (771)..(771): n is a, c, g, or t
  misc_feature (775)..(775): n is a, c, g, or t
  misc_feature (783)..(783): n is a, c, g, or t
  misc_feature (821)..(821): n is a, c, g, or t
  misc_feature (863)..(863): n is a, c, g, or t
  misc_feature (889)..(889): n is a, c, g, or t
  misc_feature (895)..(897): n is a, c, g, or t
  misc_feature (900)..(901): n is a, c, g, or t
  misc_feature (911)..(911): n is a, c, g, or t
  misc_feature (919)..(919): n is a, c, g, or t
  misc_feature (925)..(926): n is a, c, g, or t
  misc_feature (940)..(940): n is a, c, g, or t
  misc_feature (949)..(951): n is a, c, g, or t
  misc_feature (959)..(959): n is a, c, g, or t
  misc_feature (1004)..(1005): n is a, c, g, or t
  misc_feature (1009)..(1009): n is a, c, g, or t
  misc_feature (1033)..(1033): n is a, c, g, or t
  misc_feature (1073)..(1073): n is a, c, g, or t
  misc_feature (1086)..(1086): n is a, c, g, or t
  misc_feature (1091)..(1091): n is a, c, g, or t
  misc_feature (1118)..(1119): n is a, c, g, or t
  misc_feature (1126)..(1127): n is a, c, g, or t
  misc_feature (1151)..(1151): n is a, c, g, or t
  misc_feature (1195)..(1195): n is a, c, g, or t
  misc_feature (1209)..(1209): n is a, c, g, or t
  misc_feature (1218)..(1218): n is a, c, g, or t
  misc_feature (1220)..(1220): n is a, c, g, or t
  misc_feature (1275)..(1275): n is a, c, g, or t
  misc_feature (1277)..(1277): n is a, c, g, or t
  misc_feature (1281)..(1281): n is a, c, g, or t
  misc_feature (1300)..(1300): n is a, c, g, or t
  misc_feature (1328)..(1328): n is a, c, g, or t
  misc_feature (1331)..(1331): n is a, c, g, or t
  misc_feature (1379)..(1379): n is a, c, g, or t
  misc_feature (1386)..(1386): n is a, c, g, or t
  misc_feature (1388)..(1388): n is a, c, g, or t
  misc_feature (1390)..(1391): n is a, c, g, or t
  misc_feature (1394)..(1396): n is a, c, g, or t
  misc_feature (1398)..(1398): n is a, c, g, or t
  misc_feature (1411)..(1411): n is a, c, g, or t
  misc_feature (1416)..(1416): n is a, c, g, or t
  misc_feature (1429)..(1429): n is a, c, g, or t
  misc_feature (1452)..(1452): n is a, c, g, or t
ggttcgattctggctcaggatgaacgctngcggcgtgcttaacacatgcaagtcgaacggnatnctggnngcctnnctgccnggnngagagtggcgaacgggtgagtnatgcgtgaccaacctgccctgtgcaccggaatagctcctggaaacgngtggtaataccnnatctccnccnaccgcatggtggnntgggaaatgctttttgcggcatgggatggngtcgcgtcctatcagcttgttggcggggtgatggcccaccaaggcgttgacgggnagccggcctgagagggtgaccggccacnttgggactgagatacggccnnnnctcctacgggaggcagcnntggggaatattgcacaatgggcgcaagcctgatgcagcgacgccgcgtgcgggatggaggcttcgggttgtnaaccgcntttgttcnagggcnaggcacggcttcgggccgngttgagtggattgttcgaataagcaccggctaactacgtgccagcagccgcggtnatacgtagggtgcnagcgttatccggatttnttgggcgtnaagggctcgtaggcggttcgtcgcgtccggtgtgaaagtccatcgctaacggtggatctgcgccgggtacgggcgggctggagtgcggtaggggagactggaattcccggtgtaacggtggaatgtgtagatatcgggaagaacaccaatggcgaaggcaggtctctgggcngntactgacgctgaggagcnaaagcgtggggagcgaacaggattngatnccctggtngtccacgccgtaaacggtggatgctggatgtggggccntttccacgggtcctgtgtcggagccaacgcgttaagcatccngcctggggagtacggccgcaaggctnaaactnnnagnnattgacgggngccgcacnagcggnngagcatgcggattnattcgatgnnncgcgaagnaccttacctgggcttgacatgtgccggatcgccccggaaacgggnngtgncttcggggccggttcacaggtggngcatggtcgtcgtcagctcgtgtcgtgagatgttgggttnagtcccgcaacgngcgcnaccctcgccgcatgttgccagcgggtnntgccggnnactcatgtgggaccgccggggtcnactcggaggaaggtggggatgacgtcagatcatcatgccccttncgtccagggcttcncgcatgctncnatggccggtacaacgcgatgcgacacggtgacgtggggcggatcgctgaaaaccngnctcngttcggatcgcagtctgcnactcgactgcgtgaaggcggagtcgctngtnatcgcggatcagcaacgccgcggtgaatgcgttcccgggccttgtacncaccgcncntnnagnnntnaaagtgggtagcncccgnagccggtggcccnaccctcgtggggggagccgtctnaggtgag

SEQ ID NO: 10 - DNA, 1420 bp, Bifidobacterium animalis subsp. animalis
caggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacgcggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacc

SEQ ID NO: 11 - DNA, 1488 bp, Bifidobacterium bifidum
gatgaacgctggcggcgtgcttaacacatgcaagtcgaacgggatccatcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctg

SEQ ID NO: 12 - DNA, 1520 bp, Bifidobacterium breve
ttcgattctggctcaggatgaacgctggcggcgtgcttaacacatgcaagtcgaacgggatccatcgggctttgcttggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgcgggagggagccgtctaaggtgaggctcgtgattgggactaagtcgtaacaaggtagccgtaccggaaggtgcggctggatcacctcctta

SEQ ID NO: 13 - DNA, 1445 bp, Bifidobacterium catenulatum
tgaacgctggcggcgtgcttaacacatgcaagtcgaacgggatccaggcagcttgctgcctggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccccttgtgggatggagccgtctaaggtgagactcgtgattggg

SEQ ID NO: 14 - DNA, 1464 bp, Bifidobacterium longum subsp. longum
tggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgta

SEQ ID NO: 15 - DNA, 1513 bp, Bifidobacterium pseudocatenulatum
gtttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgtggatggagccgtctaaggtgagactcgtgattgggactaagtcgtaacaaggtagccgtaccggaaggtgcggctggatcacc

SEQ ID NO: 16 - DNA, 1475 bp, Bifidobacterium pseudolongum
  misc_feature (1)..(2): n is a, c, g, or t
  misc_feature (50)..(50): n is a, c, g, or t
  misc_feature (55)..(55): n is a, c, g, or t
  misc_feature (57)..(57): n is a, c, g, or t
  misc_feature (61)..(61): n is a, c, g, or t
  misc_feature (95)..(95): n is a, c, g, or t
  misc_feature (137)..(137): n is a, c, g, or t
  misc_feature (207)..(207): n is a, c, g, or t
  misc_feature (236)..(236): n is a, c, g, or t
  misc_feature (267)..(268): n is a, c, g, or t
  misc_feature (303)..(303): n is a, c, g, or t
  misc_feature (334)..(335): n is a, c, g, or t
  misc_feature (400)..(400): n is a, c, g, or t
  misc_feature (470)..(470): n is a, c, g, or t
  misc_feature (506)..(506): n is a, c, g, or t
  misc_feature (525)..(525): n is a, c, g, or t
  misc_feature (724)..(724): n is a, c, g, or t
  misc_feature (744)..(744): n is a, c, g, or t
  misc_feature (819)..(819): n is a, c, g, or t
  misc_feature (865)..(865): n is a, c, g, or t
  misc_feature (877)..(877): n is a, c, g, or t
  misc_feature (905)..(905): n is a, c, g, or t
  misc_feature (911)..(913): n is a, c, g, or t
  misc_feature (948)..(948): n is a, c, g, or t
  misc_feature (1005)..(1005): n is a, c, g, or t
  misc_feature (1118)..(1118): n is a, c, g, or t
  misc_feature (1133)..(1133): n is a, c, g, or t
  misc_feature (1136)..(1136): n is a, c, g, or t
  misc_feature (1150)..(1150): n is a, c, g, or t
  misc_feature (1169)..(1169): n is a, c, g, or t
  misc_feature (1276)..(1276): n is a, c, g, or t
  misc_feature (1367)..(1367): n is a, c, g, or t
  misc_feature (1386)..(1387): n is a, c, g, or t
  misc_feature (1393)..(1393): n is a, c, g, or t
  misc_feature (1428)..(1428): n is a, c, g, or t
  misc_feature (1453)..(1453): n is a, c, g, or t
nntttgtggagggttcgattctggctcaggatgaacgctggcggcgtgcntaacncntgcnagtcgaacgggatccatcaagcttgcttggtggngagagtggcgaacgggtgagtaatgcgtgaccgacctgcccnatacaccggaatagctcctggaaacgggtggtaatgccggatgttccagttgatcgcatggtcttctggnaaagctttcgcggtatgggatggggtcgngtcctatcagcttgacggcggggtaacggcnnaccgtggcttcgacgggtagccggcctgagagggngaccggccacattgggactgagatacggccnngactcctacgggaggcagcagtggggaatattgcacaatgggcgcaagcctgatgcagcgacgcngcgtgagggatggaggccttcgggttgtaaacctcttttatcggggagcaagcgagagtgagtttacccnttgaataagcaccggctaactacgtgccagcagccncggtaatacgtagggtgcnagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggcngttactgacgctgaggagcnaaagcgtggggagcgaacaggattagataccctggtagtccacgccgtaaacggtggatgctggatgtggggccngttccacgggttccgtgtcggagctaacgcgttaagcatcccgccnggggagtacggncgcaaggctaaaactcaaagaaattgangggggnnngcacaagcggcggagcatgcggattaattcgatgnaacgcgaagaaccttacctgggcttgacatgttcccgacggtcgtagagatacggcntcccttcggggcgggttcacaggtggtgcatggtcgtcgtcagctcgtgtcgtgagatgttgggttaagtcccgcaacgagcgcaaccctcgccccgtgttgccagcggattntgccgggaactcacnggncaccgccggggttnactcggaggaaggtggggntgacgtcagatcatcatgccccttacgtccagggcttcacgcatgctacaatggccggtacaacgggatgcgacgcggcgacgcggagcggatccctgaaaaccggnctcagttcggatcgcagtctgcaactcgactgcgtgaaggcggagtcgctagtaatcgcgaatcagcaacgtcgcggtgaatgcgttcccnggccttgtacacaccgccnntcaagncatgaaagtgggcagcacccgaagccggtggcctnaccccttgtgggatggagccgtctnaggtgaggctcgtgattgggtc

SEQ ID NO: 17 - DNA, 1478 bp, Lactobacillus acidophilus
tcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggc

SEQ ID NO: 18 - DNA, 1520 bp, Lactobacillus antri
ttgatcctggctcaggatgaacgccggcggtgtgcctaatacatgcaagtcgagcgcactggcccaactgaaatgacgtgcttgcacagaatggacgttggattcccagtgagcggcggacgggtgagtaacacgtgggcaacctgccccaaagcgggggataacatttggaaacagatgctaataccgcataagttggaaaaccacatggttttcccatcaaagatggtttcggctatcgctttgggatgggcccgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtaagagtaactgtttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtwaacgatgagtgctaggtgttggagggtttccgcccttcagtgccgaagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaacctcagagatgaggcgttcccttcggggacgcaaagacaggtggtgcatggtcgtcgtcagctcgtgtcgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatggccggtacaacgagcagctaacccgcgagggtgtgcaaatctcttaaagccggtctcagttcggactgcagtctgcaactcgactgcacgaagtcggaatcgctagtaatcgcggatcagcatgccgcggtgaatacgttcccgggccttgtacacaccgcccgtcacaccatggaaktttgcaatgcccaaagtcagtggcctaaccattatggagggasctgcctaaggcagggcagatgactggggtgaagtcgt

SEQ ID NO: 19 - DNA, 1513 bp, Lactobacillus brevis
  misc_feature (570)..(570): n is a, c, g, or t
  misc_feature (714)..(714): n is a, c, g, or t
  misc_feature (789)..(789): n is a, c, g, or t
  misc_feature (957)..(957): n is a, c, g, or t
taagatgagagtttgatcctggctcaggacgaacgctggcggcatgcctaatacatgcaagtcgaacgagcttccgttgaatgacgtgcttgcactgatttcaacaatgaagctagtggcgaactggtgagtaacacgtgggaaatctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcnagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcngtggaatgcgtagatatatggaagaacaccagtggcgaaggcggctgtctagtctgtaactgacgctgaggctcnaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggccngcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagtcgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattaggg

SEQ ID NO: 20 - DNA, 1517 bp, Lactobacillus casei
tcctggctcaggatgaacgctggcggcgtgcctaatacatgcaagtcgaacgagttttggtcgatgaacggtgcttgcactgagattcgacttaaaacgagtggcggacgggtgagtaacacgtgggtaacctgcccttaagtgggggataacatttggaaacagatgctaataccgcataaatccaagaaccgcatggttcttggctgaaagatggcgtcaagctatcgcttttggatggacccgcggcgtattagctagttggtgaggtaacggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcattgagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaag

SEQ ID NO: 21 - DNA, 1564 bp, Lactobacillus coleohominis
agtttgattctggctcaggatgaacgccggcggtgtgcctaatacatgcaagtcgaacgcgttggcccgactgattgatgatgcttgcatcagattgacgacggtttactaacgagtggcggacgggtgagtaacacgtaggcaacctgcccagaagcgggggacaacatttggaaacaagtgctaataccgcataacaacgaaaaccgcctggttttcgtttaaaagatggtttcggctatcacttctggatgggcctgcggcgcattagctagttggtaaggtaacggcttaccaaggcagtgatgcgtagccgagttgagagactgatcggccacaatggaactgagacacgggccatactcctacgggaggcagcagtagggaatcttccacaatgggcgcaagcctgatggagcaacaccgcgtgagtgaagaagggtttcggctcgtaaaactctgttgttgaagaagaacgtgcgtgagagtaactgttcacgcagtacggtattcaaccagaaagtcacggctaactacgtgccagcagccgcggtaatacgtaggtggcaagcgttatccggatttattgggcgtaaagcgagcgcaggcggttttctaagtctgatgtgaaagccttcggcttaaccggagaagggcatcggaaactggataacttgagtgcaagagaggacagtggaactccatgtgtagcggtggaatgcgtagatatatggaagaacaccagtggcgaaggcggctgtctagcttgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaacctcagagatgaggcgttcccttcggggacgcaatgacaggtggtgcatggtcgtcgtcagctcgtgttgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatgggcggtacaacgagcagcgaactcgcgagggtaagctaatctcttaaaaccgttctcagttcggactgcagtctgcaactcgactgcacgaagtcggaatcgctagtaatcgcggatcagcatgccgcggtgaatacgttcccgggccttgtacacaccgcccgtcacaccatgggagtttgcaatgcccaaagccggtggcctaaccttcgggaaggagccgtctaaggcagggcagatgactggggtgaagtcgtaacaaggtagccgtaggagaacctgcggcttgatcaccttcat

SEQ ID NO: 22 - DNA, 1518 bp, Lactobacillus crispatus
gacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcgtaagctgtcgctatgggatggccccgcggtgcattagctagttggtaaggtaaaggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgccatttgtagagatacaaagttcccttcggggacgctaagacaggtggtgcatggctgtcgtcagctcgtgtcgtgagatgttgggttaagtcccgcaacgagcgcaacccttgttattagttgccagcattaagttgggcactctaatgagactgccggtgacaaaccggaggaaggtggggatgacgtcaagtcatcatgccccttatgacctgggctacacacgtgctacaatgggcagtacaacgagaagcgagcctgcgaaggcaagcgaatctctgaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggcagggcagatgactggggtgaagtcgtaacaaggtagccgtaggagaactgc

SEQ ID NO: 23 - DNA, 1734 bp, Lactobacillus curvatus
gacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcgtaagctgtcgctatgggatggccccgcggtgcattagctagttggtaaggtaaaggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccatggaagatattgcaaatcccgagcgtacccggaaaattctcaaacgttacggctttaagtttaaaaagagcttaggccaaaacttcttaaccaacatcgccattttgaaacaaattgttgaagctggtgatatcaccaaagatgacgatgtgattgaaatcggccccggaatcggttctttaacggaacaaattgccagaaaagcgcaccaggtcttgagttttgaaattgatgaacggttaatgccggtcttaaaagatactttgaatcactaccataacgtcacgattttaaaccaagatattcttgaagcggatttaaaaacaattatcgctgaacaattcgatggtaagcataatctaaaaatcgttgcgaacttaccttattacattacgacgccaatcatgttgcacttacttgaagcaggattgccaattgattgcatggtcttaatgatgcaaaaggaagtggcagaacggatcaacgctgaacctggttcaaaggcttacggctcattaagtattgccgttcaacttcattcagaagtgaacttagcctttattgtgcccaagacagcttttatgccacaaccgaatgttgattcagcgattgttgaattagtggggcgcaaagcaccgcttgtaacagttgctaatcaaaccttatttgatcaattggttcgtggcgcatttgcacaacgccgaaagacattatggaataacttgcaaaatcaatttggtaaacaagatgaagtcaaagcagctttaacaacagccttagaagcagtcgagattgcaccgagcgctcgtgctgaacaattgagtatccaacaatttgcccaattaagcgatgtgctgaataatcagccaattttttctaaaaaagctaagtaa

SEQ ID NO: 24 - DNA, 1515 bp, Lactobacillus delbrueckii
gaacgctggcggcgtgcctaatacatgcaagtcgagcgagctgaattcaaagatcccttcggggtgatttgttggacgctagcggcggatgggtgagtaacacgtgggcaatctgccctaaagactgggataccacttggaaacaggtgctaataccggataacaacatgaatcgcatgattcaagtttgaaaggcggcgcaagctgtcactttaggatgagcccgcggcgcattagctagttggtggggtaaaggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgcgctacacctagagataggtggttcccttcggggacgcagagacaggtggtgcatggctgtcgtcagctcgtgtcgtgagatgttgggttaagtcccgcaacgagcgcaacccttgtctttagttgccatcattaagttgggcactctagagagactgccggtgacaaaccggaggaaggtggggatgacgtcaagtcatcatgccccttatgacctgggctacacacgtgctacaatgggcagtacaacgagaagcgaacccgcgagggtaagcggatctcttaaagctgttctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgggataaccttttaggagtcagccgcctaaggcagggcagatgactggggtgaagtcgtaacaaggtagccgtaggagaactgcg

SEQ ID NO: 25 - DNA, 1502 bp, Lactobacillus fermentum
cggcggtgtgcctaatacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcgaactcgcgagggcaagcaaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgtaacacccaaagtcggtggggtaaccttttaggagccagccgcctaaggtgggacagatgattagggtgaagtcgtaacaag

SEQ ID NO: 26 - DNA, 1573 bp, Lactobacillus gasseri
gaaaatgagagtttgatcctggctcaggacgaacgctggcggcgtgcctaatacatgcaagtcgagcgagcttgcctagatgaatttggtgcttgcaccagatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctctgaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaagtcgtaacaaggtagccgtaggagaacctgcggctggatcacctccttt

SEQ ID NO: 27 - DNA, 1487 bp, Lactobacillus johnsonii
ggcggcgtgcctaatacatgcaagtcgagcgagcttgcctagatgattttagtgcttgcactaaatgaaactagatacaagcgagcggcggacgggtgagtaacacgtgggtaacctgcccaagagactgggataacacctggaaacagatgctaataccggataacaacactagacgcatgtctagagtttgaaagatggttctgctatcactcttggatggacctgcggtgcattagctagttggtaaggtaatggcttaccaaggcgatgatgcatagccgagttgagagactgatcggccacattgggactgagacacggcccaaactcctacgggaggcagcagtagggaatcttccacaatggacgaaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaacgcttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcatgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctcttaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaa

SEQ ID NO: 28 - DNA, 1507 bp, Lactobacillus harbinensis
gctggcggcgtgcctaatacatgcaagtcgaacgaggtttggtcagtttgcggtggtgcttgcatcaccaattaccgatcaaaccgagtggcggacgggtgagtaacacgtgggtaacctgcccttcagcaggggataacatttggaaacagatgctaataccgtataaccacggagaccgcatggtctccgggtaaaagatggcgcaagctatcactgaaggatggacccgcggcgtattagccagttggtggggtaacggcctaccaaagcgatgatacgtagccgacctgagagggtaatcggccacattgggactgagacacggcccaaactcctacgggaggcagcagtagggaatcttccacaatggacgcaagtctgatggagcaacgccgcgtgagtgaagaaggctttcgggtcgtaaaactctgttattgaagaagaacgtgtgtgacagtaactggtcatgcagtgacggtattcaatcagaaagtcacggctaactacgtgccagcagccgcggtaatacgtaggtggcaagcgttgtccggatttattgggcgtaaagcgagtgcaggcggtcttttaagtctgatgtgaaagccttcggcttaaccgaagaagggcatcggaaactgggagacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacaggattagataccctggtagtccacgccgtaaacgatgaatactaagtgttggagggtttccgcccttcagtgctgcagctaacgcattaagtattccgcctggggagtacgaccgcaaggttgaaactcaaaggaattgacgggggcccgcacaagcggtggagcatgtggtttaattcgaagcaacgcgaagaaccttaccaggtcttgacatcttctgccaggctgagagatcagctgttcccttcggggacagaatgacaggtggtgcatggttgtcgtcagctcgtgtcgtgagatgttgggttaagtcccgcaacgagcgcaacccttatgatcagttgccagcattcagttgggcactctggtcagactgccggtgacaaaccggaggaaggcggggatgacgtcaaatcatcatgccccttatgacctgggctacacacgtgctacaatgggtggtacaacgagcagcgagaccgcgaggtcaagcgaatctctaaaaaccatcctcagttcggattgcaggctgcaactcgcctgcatgaagctggaatcgctagtaatcgcggatcagcacgccgcggtgaatacgttcccgggccttgtacacaccgcccgtcacaccatgagagtttgtaacacccaaagccggtgagacaaccgcaaggagtcagccgtctaaggtgggacaaatgattagggtgaagtcgtaaacaaggtaaca

SEQ ID NO: 29 - DNA, 1568 bp, Lactobacillus mucosae
agagtttgatcctggctcaggatgaacgccggcggtgtgcctaatacatgcaagtcgaacgcgttggcccaactgattgaacgtgcttgcacggacttgacgttggtttaccagcgagtggcggacgggtgagtaacacgtaggtaacctgccccaaagcgggggataacatttggaaacagatgctaataccgcataacaatttgaatcgcatgattcaaatttaaaagatggcttcggctatcactttgggatggacctgcggcgcattagcttgttggtagggtaacggcctaccaaggctgtgatgcgtagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttagagaagaacgtgcgtgagagcaactgttcacgcagtgacggtatctaaccagaaagtcacggctaactacgtgccagcagccgcggtaatacgtaggtggcaagcgttatccggatttattgggcgtaaagcgagcgcaggcggtttgataagtctgatgtgaaagcctttggcttaaccaaagaagtgcatcggaaactgtcagacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatggacggtacaacgagtcgcgaactcgcgagggcaagctaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgcaacacccaaagtcggtggggtaacccttcggggagctagccgcctaaggtggggcagatgattagggtgaagtcgtaacaaggtagccgtaggagaacctgcggctggatcacctcct

SEQ ID NO: 30 - DNA, 1519 bp, Lactobacillus pentosus
gacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtagggagaac

SEQ ID NO: 31 - DNA, 1517 bp, Lactobacillus plantarum
ckaacgcyggcggcgtgcctaatacatgcaagtcgaacgaactctggtattgattggtgcttgcatcatgatttacatttgagtgagtggcgaactggtgagtaacacgtgggaaacctgcccaggaagcgggggataacacctggaaacakatgctaataccgcataacaacttggaccgcatggtccgagtttgaaagatggcttcggctatcacttttggatggycccgcggcgtattakctakatggtggggtaacggctcaccatggcaatgatacgtacccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtatatatatggaasaacaccagtggcgaaggcggctgtctggttctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaa

SEQ ID NO: 32 - DNA, 1535 bp, Lactobacillus reuteri
agagtttgatcctggctcaggatgaacgccggcagtgtgcctaatacatgcaagtcgtacgcactggcccaactaattgatggtgcttgctgaattgacgatggatcaccagtgagtggcggacgggtgagtaacacgtaggtaacctgccccggagcggggaataacatttggaaacagatgctaataccgcataacaacaaaagccgcatggtttttctggaaagatggctttggctatcactctgggatggacctgcggtgcatttagctagttggtaaggtaacggcttacccaaggcgatgatgcatagccgagttgagagactgatcggccacaatgggaactgagacacggtccataacttctacgggaggcagcagtagggaatcttccacaatgggcgcaagctgatggagcaacaccgcgttattaagaaagggtttcggccgcttaaactctgttgttggagaagaacgtgcgttagagtaactgttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtggaaactcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgcctgttctaacgcattaatgcactccgcctggggagtacgaccgcaaggttgaaactcaaaggaattgacgggggcccgcacaagcggtgaagcatgtggtttaattcgaagctacgcgaagaaccttaccaggtcttgacatcttgcgctaaccttagagataaggcgttcccttcggggacgttaatgacaggtggtgcatggtcgtcgtcagctcgtgtcgtgagatgttgggttaagtcccgcaacgagcgcaacccttgttactagttgccagcattaagttggggactctagtgagactgccggtgacaaaccggaggaaggtggggacgacgtcagatcatcatgccccttatgaccctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagttcggtggcctaacctttatggacgggtaccctaaggcgggacagatgatctggggtgaagtcgtaacaaggta

SEQ ID NO: 33 - DNA, 1521 bp, Lactobacillus rhamnosus
  misc_feature (407)..(407): n is a, c, g, or t
  misc_feature (655)..(655): n is a, c, g, or t
  misc_feature (1476)..(1476): n is a, c, g, or t
grtsaacgctsgcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactctacgggaggcagcagtagggaatcttccacaatggacgcaagtctgatggagcaacgccgcgtgagtnaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtncagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggncaaatgattagggtgaagtcgtaacaaggtagccgtaggagaacc

SEQ ID NO: 34 - DNA, 1561 bp, Lactobacillus sake
aagtttgattattgctcaggacgaacgctggcggcgtgcctaatacatgcaagtcgaacgcactctcgtttagattgaaggagcttgctcctgattgataaacatttgagtgagtggcggacgggtgagtaacacgtgggtaacctgccctaaagtgggggataacatttggaaacagatgctaataccgcataaaacctaacaccgcatggtgtagggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaatgtatctgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggatgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagccggaatcgctagtaatcgcggatcagcatgccgcggtgaatacgttcccgggccttgtacacaccgcccgtcacaccatgagagtttgtaacacccaaagccggtgaggtaacccttcggggagccagccgtctaaggtgggacagatgattagggtgaagtcgtaacaaggtagccgtagagaacctgcggctggatcacctc

SEQ ID NO: 35 - DNA, 1542 bp, Lactobacillus salivarius
agagtttgatcctggctcaggacgaacgctggcggcgtgcctaatacatgcaagtcgaacgaaactttcttacaccgaatgcttgcrttcatcgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggtccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaaggtagccgtaggagaacctgcggctg

SEQ ID NO: 36 - DNA, 1972 bp, Lactobacillus paracasei
agagtttgatcctggctcaggacgaacgctggcggcgtgcctaatacatgcaagtcgaacgaaactttcttacaccgaatgcttgcrttcatcgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggtccaaactcctacgggaggcagcagtagggaatcttccacaatggacgcaagtctgatggagcaacgccgcgtgagtgaagaaggtcttcggatcgtaaaactctgatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacc

SEQ ID NO: 37 - DNA, 1565 bp, Lactobacillus kisonensis
agagtttgatcctggctcaggacgaacgctggcggcgtgcctaatacatgcaagtcgaacgcgtcttggttatcgaagggaagtgcttgcatttccttgacttaacattgagacgagtggcgaactggtgagtaacacgtgggtaacctgcccttgaagtaggggataacacttggaaacaggtgctaataccgtataacaaccaaagccccatgactttggtttaaaagatggcttcggctatcactttaggatggacccgcggcgtattagcttgttggtgaggtaacggctcaccaaggcaatgatacgtagccgacctgagagggtaatcggccacattgggactgagacacggcccaaactcctacgggaggcagcagtagggaatcttccacaatggacgaaagtctgatggagcaacgccgcgtgagtgatgaagggtttcggctcgtaaaactctgttgttggagaagaacgggtgtgagagtaactgttcacatcgtgacggtatccaaccagaaagccacggctaactacgtgccagcagccgcggtaatacgtaggtggcaagcgttgtccggatttattgggcgtaaagcgagcgcaggcggttttttaggtctgatgtgaaagccttcggcttaaccggagaagtgcatcggaaaccgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgatgctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattgttagttgccagcatttagttgggcactctagcaagactgccggtgacaaaccggaggaaggtggggatgacgtcaaatcatcatgccccttatgacctgggctacacacgtgctacaatggacggtacaacgagtcgcgaaaccgcgaggtcaagctaatctcttaaagccgttctcagttcggattgtaggctgcaactcgcctacatgaagttggaatcgctagtaatcgtggatcagcatgccacggtgaatacgttcccgggccttgtacacaccgcccgtcacaccatgagagtttgtaacacccaaagccggtgaggtaaccttcggggaccagccgtctaaggtgggacagatgattagggtgaagtcgtaacaaggtagccgtaggagaacctgcggctggatcacctcc

SEQ ID NO: 38 - DNA, 1522 bp, Lactobacillus paralimentarius
gacgaacgctggcggcatgcctaatacatgcaagtcgaacgaaccatcctgaagattgaagcttgcttcatgattcagaccttggtgagtggcggacgggtgagtaacacgtgggtaacctgcccagaagtgggggataacatttggaaacaagtgctaataccgcataacaacttagatcacatgatctttgtttaaaagatggttttgctatctcttctggatggacccgcggcgtattagctagttggtgaggtaatagctcaccaaggcgatgatacgtagccgacctgagagggtaatcggccacattgggactgagacacggcccaaactcctacgggaggcagcagtagggaatcttccacaatggacgaaagtctgatggagcaatgccgcgtgagtgaagaaggttttcggatcgtaaaactctgttgttgaagaagaacatatgtgagagtaactgttcacgtactgacggtattcaaccagaaagccacggctaactacgtgccagcagccgcggtaatacgtaggtggcaagcgttgtccggatttattgggcgtaaagagaatgtaggcggtttattaagtttgaagtgaaagccctcggctcaaccgaggaagtgcttcgaaaactggtaaacttgagtgcagaagaggaaagtggaactccatgtgtagcggtggaatgcgtagatatatggaagaacaccagtggcgaaggcggctttctggtctgtaactgacgctgagattcgaaagcatgggtagcaaacaggattagataccctggtagtccatgccgtaaacgatgagtgctaagtgttggagggtttccgcccttcagtgctgcagctaacgcattaagcactccgcctggggagtacgatcgcaagattgaaactcaaaggaattgacgggggcccgcacaagcggtggagcatgtggtttaattcgaagcaacgcgaagaaccttaccaggtcttgacataccatgaaaagcttagagataagtctttcccttcggggacatggatacaggtggtgcatggttgtcgtcagctcgtgtcgtgagatgttgggttaagtcccgcaacgagcgcaacccttattatcagttgccagcattcagttgggcactctggtgagactgccggtgataaaccggaggaaggtggggacgacgtcaaatcatcatgccccttatgacctgggctacacacgtgctacaatggtcggtacaacgtgctgcgaactcgcgagggcaagcaaatcacttaaaaccgatctcagttcggattgtaggctgcaactcgcctacatgaagctggaatcgctagtaatcgcggatcagcatgccgcggtgaatacgttcccgggccttgtacacaccgcccgtcacaccatgagagtttgtaacacccaaagtcggtggggtaacccttcggggaactagccgcctaaggtgggacaaatgattagggtgaagtcgtaacaaggtagccgtaggagaacctgc

SEQ ID NO: 39 - DNA, 1554 bp, Lactobacillus perolens
tggctcaggacgaacgctggcggcgtgcctaatacatgcaagtcgaacgaggtttgatcagtttgcggtggtgcttgcatcaccaattaccgattaaaccgagtggcggacgggtgagtaacacgtgggtaacctgcccttcagcaggggataacatttggaaacagatgctaataccgtataaccacggagaccgcatggtctccgggtaaaagatggcgaaagctatcactgagggatggacccgcggcgtattagccagttggtggggtaatggcctaccaaagcgatgatacgtagccgacctgagagggtaatcggccacattgggactgagacacggcccaaactcctacgggaggcagcagtagggaatcttccacaatggacgcaagtctgatggagcaacgccgcgtgagtgaagaaggctttcgggtcgtaaaactctgttattgaagaagaacgtgtgtgagagtaactgctcatgcagtgacggtattcaaccagaaagtcacggctaactacgtgccagcagccgcggtaatacgtaggtggcaagcgttgtccggatttattgggcgtaaagcgagtgcaggcggttttttaagtctgatgtgaaagccttcggcttaaccgaagaagtgcatcggaaagtgggaaacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacaggattagataccctggtagtccacgccgtaaacgatgaatactaagtgttggggggtttccgcccctcagtgctgcagctaacgcattaagtattccgcctggggagtacgaccgcaaggttgaaactcaaaggaattgacgggggcccgcacaagcggtggagcatgtggtttaattcgaagcaacgcgaagaaccttaccaggtcttgacatcttctgccaagctgagagatcagccgttccttcggggacagaatgacaggtggtgcatggttgtcgtcagctcgtgtcgtgagatgttgggttaagtcccgcaacgagcgcaacccttatgattagttgccagcattcagttgggcactctagtcagactgccggtgacaaaccggaggaaggcggggatgacgtcaaatcatcatgccccttatgacctgggctacacacgtgctacaatgggtggtacaacgagcagcgagaccgcgaggtcaagcgaatctctaaaaaccatcctcagttcggattgtaggctgcaactcgcctacatgaagctggaatcgctagtaatcgcggatcagcacgccgcggtgaatacgttcccgggccttgtacacaccgcccgtcacaccatgagagtttgtaacacccaaagccggtaggacaaccgcaaggagtcagccgtctaaggtgggacaaatgattagggtgaagtcgtaacaaggtagccgtaggagaacctgcggctggatcacctcctttct

SEQ ID NO: 40 - DNA, 1428 bp, Lactobacillus apis
tgcagtcgagcgagcaagttaaggaatacttcggtaggaatttaatagcgcgagcggcggatgggtgagtaacacgtgggcaacctgccctttagcttgggataccacttggaaacaggtgctaataccaaataagaagtaagagcgcatgctcaagctatgaaaggcggctttcgagctgtcactaaaggatgggcccgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggacatgggtagtaactgatctatgtttgacggtaatcaaccagaaagtcacggctaactacgtgccagcagccgcggtaatacgtaggtggcaagcgttgtccggatttattgggcgtaaagcgaacgcaggcgggagaacaagtcagctgtgaaagccctcggcttaaccgaggaacggcaactgaaactgtttttcttgagtgcagaagaggagagtggaactccatgtgtagcggtgaaatgcgtagatatatggaagaacaccagtggcgaaggcggctctctggtctgtaactgacgctgaggttcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttaaaactcaaaggaattgacgggggcccgcacaagcggtggagcatgtggtttaattcgaagcaacgcgaagaaccttaccaggtcttgacatctagtgccaaccctagagatagggtgtttccttcgggaacactaagacaggtggtgcatggctgtcgtcagctcgtgtcgtgagatgttgggttaagtcccgcaacgagcgcaacccttattattagttgccagcattaagttgggcactctaatgagactgccggtgacaaaccggaggaaggtggggacgacgtcaagtcatcatgccccttatgacctgggctacacacgtgctacaatggttagtacaacgaggagcgaacctgtgaaggcaagcgaatctcttaaagctaatctcagttcggattgcactctgcaactcgagtgcatgaagctggaatcgctagtaatcgcggatcagcatgccgcggtgaatacgttcccgggccttgtacacaccgcccgtcacaccatgagagtttgtaatacccaaagccggtgagataacctgtaaaggagtcagc

SEQ ID NO: 41 - DNA, 1460 bp, Lactobacillus ghanensis
gcggcgtgcctaatacatgcaagtcgaacgaagttgttttaactggtgcttgcaccgacttaaacaactgagtggcgaacgggtgagtaacacgtgggtaacctgccccaaagcgggggataacatttggaaacagatgctaataccgcataaccacagaaacctcatggtttctgtgtcaaagatggtttcggctatcactttgggatggacccgcggcgtattagctagttggtaaggtaacggcttaccaaggcaatgatacgtagccgaactgagaggttgatcggccacattgggactgagacacggcccaaactcctacgggaggcagcagtagggaatcttccacaatggacgaaagtctgatggagcaacgccgcgtgagtgaagaaggttttcggatcgtaaaactctgttgtcagagaagaacgtgtgcgagagtaactgttcgtgcagtgacggtatctgaccagaaagccacggctaactacgtgccagcagccgcggtaatacgtaggtggcaagcgttgtccggatttattgggcgtaaagggaacgcaggcggttttttaagtctgatgtgaaagccttcggcttaaccgaagtcgtgcattggaaactggagaacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgctgtaaacgatgaatgctaagtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttctgcaagcctgagagatcaggtgttcccttcggggacagaatgacaggtggtgcatggttgtcgtcagctcgtgtcgtgagatgttgggttaagtcccgcaacgagcgcaacccttattgccagttgccagcattcagttgggcactctgacaagactgccggtgacaaaccggaggaaggtggggatgacgtcaaatcatcatgccccttatgacctgggctacacacgtgctacaatggacgatacaacgagttgctagaccgcgaggttaagctaatctcttaaagtcgttctcagttcggattgcaggctgcaactcgcctgcatgaagtcggaatcgctagtaatcgcggatcagcatgccgcggtgaatacgttcccgggccttgtacacaccgcccgtcacaccatgagagtttgtaacacccaaagccggtggggtaaccagtgggaaccagccgtctaaggtgggacaga

SEQ ID NO: 42 - DNA, 1464 bp, Lactobacillus dextrinicus
  misc_feature (394)..(394): n is a, c, g, or t
  misc_feature (397)..(397): n is a, c, g, or t
  misc_feature (781)..(781): n is a, c, g, or t
ctggcggcgtgcctaatacatgcaagtcgaacgagtttgcttttaatgaaggcggtgcttgcaccaactgatttaaaaataaacgagtggcggacgggtgagtaacacgtgggtaacctaccctaaagtgggggataacatttggaaacagatgctaataccgcataatatcaaaaaccacatggtttttaattgaaagacggcgtaagctgtcacttttggatggacccgcggcgtattagctagttggtgaggtaacggctcaccaaggcgatgatacgtagccgacctgagagggtgatcggccacattgggactgagacacggcccaaactcctacgggaggcagcagtagggaatcttccacaatggacgaaagtctgatggagcaacnccncgtgagtgaagaaggttttcggatcgtaaaactctgttattggagaagaacgtatttggtagtaactggccagatagtgacggtatccaatcagaaagccacggctaactacgtgccagcagccgcggtaatacgtaggtggcaagcgttgtccggatttattgggcgtaaagcgagtgcaggcggttttttaagtctgatgtgaaagccttcggcttaaccgaagaaatgcattggaaactgggaaacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacagnattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagcgccaatcctagagataggacgttcccttcggggacgctaagacaggtggtgcatggttgtcgtcagctcgtgtcgtgagatgttgggttaagtcccgcaacgagcgcaacccttattatcagttgccagcatttagttgggcactctggtgagactgccggtgacaaaccggaggaaggtggggatgacgtcaaatcatcatgccccttatgacctgggctacacacgtgctacaatggctagtacaacgagttgcgagaccgcgaggtcaagctaatctctaaaagctagtctcagttcggattgtaggctgcaactcgcctacatgaagttggaatcgctagtaatcgcggatcagcacgccgcggtgaatacgttcccgggccttgtacacaccgcccgtcacaccatgagagtttgtaacacccgaagccggtggagtaacccgtaagggagctagccgtct

SEQ ID NO: 43 - DNA, 1499 bp, Lactobacillus lactis
gacgaacgctggcggcgtgcctaatacatgcaagttgagcgatgaagattggtgcttgcaccaatttgaagagcagcgaacgggtgagtaacgcgtggggaatctgcctttgagcgggggacaacatttggaaacgaatgctaataccgcataacaactttaaacataagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaacaaggtagccgtatcggaagg

SEQ ID NO: 44 - DNA, 1510 bp, Bacteroides vulgatus
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 45 - DNA, 1529 bp, Bacteroides fragilis
acaatgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctggaacacctccttt

SEQ ID NO: 46 - DNA, 1462 bp, Faecalibacterium prausnitzii
gatcctggctcaggcgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtggccgatgtttactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtac

SEQ ID NO: 47 - DNA, 1521 bp, Eubacterium rectale
agagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacct

SEQ ID NO: 48 - DNA, 1539 bp, Streptococcus thermophilus
atgggagagtttgatcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaatggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgggtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcgagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggacagatgattggggtgaagtcgtaacaaggtagccgtatcggaaggtgcggctggatcac

SEQ ID NO: 49 - DNA, 1436 bp, Pediococcus parvulus
  misc_feature (1345)..(1345): n is a, c, g, or t
acatgcaagtcgaacgcactttcgttgaatgaattagaggtgcttgcaccgaagatgattttagctataaagtgagtggcgaacgggtgagtaacacgtgggtaacctgcccagaagtgggggataacacctggaaacagatgctaataccgcataacaaagtaaaccgcatggtttacttttaaaagatggcttcggctatcacttctggatggacccgcggcgtattagctagttggtgagataaaggctcaccaaggcagtgatacgtagccgacctgagagggtaatcggccacattgggactgagacacggcccagactcctacgggaggcagcagtagggaatcttccacaatggacgaaagtctgatggagcaacgccgcgtgagtgatgaaggctttagggtcgtaaaactctgttgttggagaagaacgtgtgtgagagtaactgctcatgcagtgacggtatccaaccagaaagccacggctaactacgtgccagcagccgcggtaatacgtaggtggcaagcgttatccggatttattgggcgtaaagcgagcgcaggcggtcttttaagtctaatgtgaaagccttcggcttaaccgaagaagtgcattggaaactggaagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaagtgttggagggtttccgcccttcagtgctgcagctaacgcattaagcattccgcctggggagtacgaccgcaaggttgaaactcaaaagaattgacgggggcccgcacaagcggtggagcatgtggtttaattcgaagctacgcgaagaaccttaccaggtcttgacatcttctgccaatctaagagattagacgttcccttcggggacagaatgacaggtggtgcatggttgtcgtcagctcgtgtcgtgagatgttgggttaagtcccgcaacgagcgcaacccttattattagttgccagcattaagttgggcactctagtgagactgccggtgacaaaccggaggaaggtggggacgacgtcaaatcatcatgccccttatgacctgggctacacacgtgctacaatggacggtacaacgagttgcgagaccgcgaggtttagctaatctcttaaaaccgttctcagttcggactgcaggctgcaactcgcctgcacgaagttggaatcgctagtaatcgcggatcancatgccgcggtgaatacgttcccgggccttgtacacaccgcccgtcacaccatgagagtttgtaacacccaaagccggtggagtaaccttc

SEQ ID NO: 50 - DNA, 1540 bp, Pediococcus lolii
caagtcgaacgaacttccgttaattgattatgacgtgcttgcactgaatgagattttaacacgaagtgagtggcggacgggtgagtaacacgtgggtaacctgcccagaagcaggggataacacctggaaacagatgctaataccgtataacagagaaaaccgcctggttttcttttaaaagatggctctgctatcacttctggatggacccgcggcgcattagctagttggtgaggtaacggctcaccaaggcgatgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggtaatagggtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctgttagtaccctgctgtcaacgataagagtgattactaagtgttggagggtttccgcccttcagtgctgcagctaacgcattaagttatccgcctggggagtacgaccgcaaggttgaaactcaaagaattgacgggggccgcacaagcgtggagcatgtggttaattcgaagtacgcgaagaacttaccaggtcttgacatctttgccaacctaagagattaggcgttccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagtcgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctttctaaggaataatacggaacc

SEQ ID NO: 51 - DNA, 1540 bp, Pediococcus acidilactici; identical to SEQ ID NO: 50

SEQ ID NO: 52 - DNA, 1492 bp, Pediococcus argentinicus
gatgaacgctggcggcgtgcctaatacatgcaagtcgaacgcacttccgtcgaatgatttcaaggtgcttgcaccgcgaatgaaaatgacatgaagtgagtggcgaacgggtgagtaacacgtgggtaacctgcccagaagtaggggataacacctggaaacagatgctaataccgtataatagagaaaaccgcatggttttcttttgaaagatggctctgctatcacttctggatggacccgcggcgtattagctagttggtgaggtaackgctcaccaaggcagtgatacgtagccgacctgagagggtaatcggccacattgggactgagacacggcccagactcctacgggaggcagcagtagggaatcttccacaatggacgaaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtttcttaagtctaatgtgaaagccttcggctcaaccgaagaagtgcattggaaactgggaaacttgagtgcagaagaggatagtggaactccatgtgtagcggtgaaatgcgtagatatatggaagaacaccagtggcgaaggcggctatctggtctgcaactgacgctgaggctcgaaagcatgggtagcgaacaggattagataccctggtagtccatgccgtaaacgatgaatgctaagtgttggagggtttccgcccttcagtgctgcagctaacgcattaagcattccgcctggggagtacgaccgcaaggttgaaactcaaaagaattgacgggggcccgcacaagcggtggagcatgtggtttaattcgaagctacgcgaagaaccttaccaggtcttgacatcttctgccaatctaagagattagacgttcccttcggggacagaatgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgat

SEQ ID NO: 53 - DNA, 1472 bp, Pediococcus claussenii
tgcaagtcgaacgcatttccgttaatagaatcagaagtgcttgcacggatagatgattttaacaatgaaatgagtggcgaacgggtgagtaacacgtgggtaacctgcccagaagagggggataacacttggaaacaggtgctaataccgcataataaagaaaaccgcatggttttcctttaaaagatggtttcggctatcacttctggatggacccgcggcgtattagctagttggtaaggtaaaggcttaccaaggcagtgatacgtacccgacctgagagggtaatcggccacattgggactgacacacggcccatactcctacgggaggcagcaatagggaatcttccacagtggacgaaagtctgatggagcaacgccgcgtgagtgaagaagggtttcggctcgtaaaactctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatactaagtgttggagggtttccgcccttcagtgctgcagctaacgcattaagtattccgcctggggagtacgaccgcaaggttgaaactcaaaagaattgacgggggcccgcacaagcggtggagcatgtggtttaattcgaagctacgcgaagaaccttaccaggtcttgacatcttctgacattctaagagattagaagttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgaaaccgcgaggtttagctaatctcttaaagccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattaggg

SEQ ID NO: 54 - DNA, 1569 bp, Pediococcus pentosaceus
  misc_feature (120)..(120): n is a, c, g, or t
agagtttgatcatggctcaggatgaacgctggcggcgtgcctaatacatgcaagtcgaacgaacttccgttaattgattatgacgtacttgtactgattgagattttaacacgaagtagntggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggtaaaggcccaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctt

SEQ ID NO: 55 - DNA, 1529 bp, Pediococcus stilesii
gatgaacgctggcggcgtgcctaatacatgcaagtcgaacgaacttccgttaattgattaagcggtacttgtaccaattgagattttaacacgaagtgagtggcgaacgggtgagtaacacgtgggtaayctgcccagaagtaggggataacacctggaaacagatgctaataccgtataatagagaaaaccgcatggttttcttttgaaagatggctctgctatcacttctggatggacccgcggcgcattagctagttggtaaggtaaaggcttaccaaggcagtgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgaatgctaagtgttggagggtttccgcccttcagtgctgcagctaacgcattaagcattccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatgatacaacgagtcgcgaaaccgcgaggtttagctaatctcttaaaatcattctcagttcggactgtaggctgcaactcgccyacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaagaaaacc

SEQ ID NO: 56 - DNA, 20 bp, Bifidobacterium longum subsp. infantis
atacagcagaaccttggcct

SEQ ID NO: 57 - DNA, 20 bp, Bifidobacterium longum subsp. infantis
gcgatcacatggacgagaac

SEQ ID NO: 58 - DNA, 27 bp, Bifidobacterium longum subsp. infantis
  misc_feature (1)..(1): FAM dye
  misc_feature (10)..(10): n = ZEN quencher
  misc_feature (11)..(11): n is a, c, g, or t
  misc_feature (25)..(25): n = IABkFQ quencher
  misc_feature (27)..(27): n is a, c, g, or t
ntttcacggantcaccggaccatacgn

SEQ ID NO: 59 - DNA, 1113 bp, Bifidobacterium longum subsp. infantis
gtgataattggaaaacgtctcggtgaagatcggttcaaattttacaaaaccgtccttgccggaaatacagatcagctgctattcgacgaggaatttaatgcatggtttgtgttcaatgtgcgtaaccttcatactttgataagagatgagcggctcaaagccaacactaaaactgtcatagataaactccctctgaagatagaaactatacggacattaaagaatttttattccaactgggttatgttcaccgacggagagtctcataaacagcttcgtcgcctagtgggaacaatcattaatactcgttatcacgccataaattacacatggccgcaaatcaataaaaattgtgacttcactacagagtatgcaaggccttatgtgtacggtatccttgctcaactggtgggtgtctctgtagaagacatttcaagaatggtctcggcatcggaaaccattaactcgttcttactgcgcgaacggcttacgctagacgatatcgaacaagtcgcgcattcaatcgaatacgcgtatcaggttgtcaaagaaatcgaagataagcatgtaggggagccgctgtacataggcaacgaattgcttgacttgcctcaggagacacgatatcccctcatcattaatctagtaaccgatggttttgccccattcgtagctgcgcttgatttcctggcgttcaatctgttgactcacccatatttagaggaagagctcaatgcaaaagcagggcaaatatcccttgaaagtcttcggttgttccctccattcacgacaataagcaggacatgcgtgcatgaaataccttttaaggaaaaaattattagaccaaggcagttggtaatttttgatttatacagcattaatcgcgacccggaagtttttcccgatccggaaaaatttaaccttgagaacactgcaagagcatactcctttggggctgcgcagcatctatgctccggaaacccgctggtgagaaaagccctggaacaagtaacgcgccaaagcgaatcactatataaatacaagatacagtcttattgcttcaaaaattcatacggttttaccgacatgaatttgtctattgaattaaaataa

SEQ ID NO: 60 - DNA, 333 bp, Bifidobacterium longum subsp. infantis
atggaacccatcaaagactggctgaccggcctcgaccgagaatccaaactgcagatcctcgccgccgtcgaagtcctgaaagaacaaggacccaacctcagacgaccactggtaggcaaaatcgaaggctccaaaatcaaatcgatgaaggaactccgacccggctccgcaggcaaaagcgaaatacgaatactgttcgtgttcgacccgaaccggcaggcgataatgctcgtcggcggagacaagcaaaacaaatggtcgaaatggtacaaaacggccataccggaagcggaagaccggtatgaggcgtggcttaaacagaacaacaaatga

SEQ ID NO: 61 - DNA, 1101 bp, Bifidobacterium longum subsp. infantis
atggacgatgcgcagtacatgggactcgcgatcgagctggcgaagcgaggggccggatacgtgaaccccaatcccatggtcggcgcggtgatcgtcaaggacgatcggatcatcgggcaggggtatcacgagatgttcggcggattgcacgccgagaggaacgcgttgcggcattgcacgcagtcacccgccggggcgacgttgtacgtcacgcttgagccgtgctgccattacggcaaaacgccgccatgcacggaggcgatcgtggaaagcggcatcgcaagggtcgtcgtcggaaccttggactgcaatcccgtggtgtccggcaagggcgtgcgcatgctcgaagaccacggcatacgggtcgatgtgggggtgctggccgacgagtgccggcatctgatacgggtgttcagcaaatacatcaccacgcatacgccgtatgtcatcatgaaatacgccatgacgatggacgggaagatagcgacccataccaatcaatcacggtggatcagcggcgaggagtcgaggcgtcgggtccatcagctccgtcgatccgtcgcggcggtcatggtgggggtgaacacggtgatcgaggacgacccgttgctgacatgccgcatggcacacggaaggaacccggttcgcgtcgtctgcgatacgcgattgaggacgcccctcacctcgcggatcgtgcagacggcgaatgacgtcaggacctatatcgccaccgcctgcgatgacgaacgcaaggcggaggactaccggcggcacggctgcgaaatactcgccgtcggaaggaagggcgaccacgtcgatctggcggacgtggtgcggcgcctgggggatatgcagatggacagcgtgctgctggaaggcgggagcgcgatgaattggagcgccctcgaacagcggatcgtcgatgaggcgcacgtgtacatcgcgccgaagatattcggcggcaccgcgaaaagcccggtgggcggccaaggggtcgccctgccttccgacgccgtcatgctccggccccgcgcctgctcccgagtgggagaggattatctggtggaaagcgaggtggtgtattcatgttcacgggaatag

SEQ ID NO: 62 - DNA, 1638 bp, Bifidobacterium longum subsp. infantis
atgactttcatcaaacagatgatgccgcgttatgtggcgtctatgaccgcaggcatcgtagcggcagcgatggccgcaacatgcgcgtttgcaccggtagcgaatgcggacgccgtttctccgacacaggaaaccatacaatcgaccggtcgccatttcatggtgtactaccgcgcgtggcgtgatgtcacgatgaagggtgtcaacaccgaccttccagacgataactggatttcgatgtacgatattccgtacggcgtcgacgtggtcaatatattcagctatgttccatcgggacaggaagaacaggcgcaaccgttctacgataagctcaaatctgattatgcaccgtacttgcattcgcgtggcatcaagctggttcggggcattgattacaccggcgtggcggtcaatggttttcgtactttcatgaaagagcagaacaaaaccgagagcgaggcgaccgaagctgattatgatgcctatgctaagcaagtaatcgataaatacatgatctccgttggcttggatggtctggatatcgacatggaggcgcacccgaatgatgccgatgtgaagatctccgacaatgtgattcgtgcactgtccaaacacatcggtcccaaatccgccaagccggataccacgatgttcctttatgacacgaatggatcttatctcaatccgttcaagaatgtggcggaatgctttgactacgtggcataccagcagtacggttcttcctccgatcgcaccgctagggccgccgccgattatcagccttatatcggcaacgagtttgtgccgggcctgacgttccccgaagaaggggacatgaacaatcgctggtatgatgccaccgagccgtatgaagaaagccatttctatcaagtggcgtcatatgtgcgtgagcataatcttggcggcatgttcgtttacgcgctcgaccgagacggtcgcaactatgatgaagacctgcgtcggattgtaccgtccaacttgctgtggaccaagaccgccattgcggagagcgagggcatggcgttggatacggcaaagactgcagcgaaccattaccttgatcgcatgtcgttgcgtcaggtgattgacgacaacgccgcatccgcggataaggcgcgggatatggtggggaaggccgctaacctatacgagacaaataaagcagtccttggtggtgactacggagaaggtttctccaacacatatgatccgacattggaagctggattattagggattgacatctccgtgttgcaacagcagattgataaatccagtgagatcattggtgccgatacggcggaatcggatgcaaaaactgcactccgtatggcacgtgatgccgccatcgatggtttgaccggcaagatctacacagcggaccaagtatcagcgtggtcgcaggcattgaaggcggcgcttgatgccaccgtgccggttccgacgccagattccaccgatcagaatggcaaccgtgacaaggttaccaatcacaaagtgcaaggccaaccgaagcaactgagtgcgaccggcatctcgacggatattatcgttgccgtaggcgtgactcttgccatcgcaggggtggctctgtctctatcacgtaagctctcctga

SEQ ID NO: 63 - DNA, 876 bp, Bifidobacterium longum subsp. infantis
atgcagtacgttaagggcaccaatcaggaactattgaccacatcaccgcagttccctgccctggtcatcgcacatcagggattcgacaaagccaggttgcattggcatgaaggcttcgaggtggtatacgtccgacgctgtcgggccacagtgatgaaaggcacccaacgttcgatgcatcatgccggtgatgtagtactgattcctccacgctgcctgcatagcatcgaacttattcaggatcttcggcaaccatcagctgtaccgcaggcgctttccgtaacgatctcgcccacggagttgctaccgtcatatccgtatattacccaaatccaacaacagctggattacgactacattggcgaaaaagatcatcaacaattgctgaaatgctgcgaacaaatgtttgcggcactgatcagtggcaagcagacacggttccttgaagcgaattcctggttctacaccatgcttaccaatgtttttgactacgccaaaccattggctgcagcgaaccaagaagacaccgaggatgaaaccatactccaggatggacgagtcattagacgaatacgccactacgtgcaacgtcattatcgcgagcccctatccaccgcacaggtgtcgcagaaattcggatattcacgagagcatttttcaaggatattcagcaaatattctggagtgacatttaaagactacgtaacgcgtgtacgtctgctggcggcctgcgatttgctcaccaacacggatatgccaatcaatcaaatcgtccgggaatccggattccccagttctcaaaccatgcgaggatcgtttgaccgcgaattcgcatgcacgcccagcgaatatcgctctcgttcgatggaagaataa

SEQ ID NO: 64 - DNA, 696 bp, Bifidobacterium longum subsp. infantis
atgaatactaacgatacttattcgaaacgtcttattgattcgctccacggcacactgatagtcagctgtcaagcatatcccggcgagccaatgcggcatcccgaaaccatggcccaagtcgctcaatcagtagtaatcggcggcgctgccgccattcgatgccaaggtcttgccgatatcagcgccatcaaaggccaagtaaaagttccagtcatcggcatatggaaggaaggcgataacggcgtatacatcacgccaacgttacgccacgccagagcctgcattatggcgggagccgacattgtggcactcgatgctacagatcggcctcggccagacggtctaactctgcagcagaccgcgcggcaactcaaaagcgagggcgcaatactgatggccgactgcggctgcatcgaagactccgatgcagccgtggatgcaggattcgacatcatctccaccacgctggctggatacaccgattcccgcgccaaaacagaaggaccggattacgagctcctcgctcaaatgctggagcggcacccccacgtaccggtgatatgcgagggtaggatacatactccgtccgatgcggccaaagccatagaaatgggtgcctgggcagccgtagtcggtaccgccatcacccaccccatgacgataacgtcatggttcgccgatgcagtaaggtcctga

SEQ ID NO: 65 - DNA, 1020 bp, Bifidobacterium longum subsp. infantis
atgagttttgcaagcataatccatcagcatacggtgatatccgtggacatcggcggcaccaaaatcgccgctggtctcatgtgcgtagacctcgacgaacaaggaagaccgcgggacatgccagcgttgattcgacggtacgtcataccgacgaaagcacgtctcggcggcacctccgtattgcaacgcgtcgttcaagccattcgccagtgtctggacgatggtgagattcctttcccactattggggatcggtgtagcgagcgcaggagtcattgacggcaacggttcggtggtatcagccacttcgttgattcccggttgggccggtattcaactgcaagccgaattatcggcaacgttcaacgtgcctgccatcgtcatcggagatgttcaggcacacgcccttggtgaagcgcactggggatgcggacgacaataccaatcactactggtcgccgccatcggcaccggtatcggaggcgcgataatcatcgacgggaaactggtacgcggcgtccacggagcctgtggccatatcggccatctgccgcatcccgacgccattggtatatcctgctcatgtggatgtgagggccatgtggagtcaatcgcctcaggtaccggcattgccgataattatcgacgtgctctgcaacagaacggacacaatactctccccaccgacatcaatggacgcgctatcgccgaactggccagtcaaggtagtacggaagccgtaaccagtatcactctggccggcacgagtctcggcgatgttcttggcggactaatcaatgcccttgatcccgatgccgtgatcctttccggctccgtagtccattctggagagctatggatgaaatctgtgaaacaaggcatccaaagacagaccttgggcattctttcccgaacaccggttttgacaggaacactcggcggcagagcccctctcatcggagcaacatcagctctgtgcgccgacctcagatcaaataaggagcagaaatatgaatactaa

SEQ ID NO: 66 - DNA, 948 bp, Bifidobacterium longum subsp. infantis
atggaagagaggcgcgagaagtcgattgcggtgactgccctgaatgagggcgggggagggaaatctcatcgctcaggtcgatattccgccgagcggcagccccatgcgtggctgttgctgttcccggccttggcgctgatcaccgtgttcaatctgcttcccctggtgcgtatcttcatcatgtcgctgcagggcgggacgttgacccgtcaacggtttgtgggaatgcgtaattttgttttcgtgttcgcggatcccgaattccgcaaggccatcgccaatacggcgctgttcgcattcgtcgtggtgtcggtcgggctggtgctgtcgatggcggtggccgtggccatcaatggaaagcttcgcggaggcagggtcttcgaaatcctgttcttcatcccctatctgacctcggtgatcgccatcggcatggtgttccggtatctgttcaacggcgattacggtcttgtcaattacgtgttgggtctgtgcggccttggaccctatgattttctcaacgatccgaagttcaatatgcccacgctcatcatcttcggcatatggtcgtccctggcgttcaacatcatcattctgttgtcgggcctgcgcggcatagacaaggaatattacaaggtggccgacatgttcggcgccacggcctgggagaagttccgccggatcacgttgccgcagatggtgccgattctgacgttcctgtccatcgtggatttcatcaattccttcaaggtgtatacggaggtgtacgccttgttcaacgggaaggccggtatcggcgatagcgccacgacggcggtgttctacgtgttcaacaagttctacgtggacaacaagtatgggcagggcatggccgcggcggtggtgctgttcctggtgattctggctttcaccatcatccaaggcatcgtattgaggaggctggcgaagtga

SEQ ID NO: 67 - DNA, 1185 bp, Bifidobacterium longum subsp. infantis
atgacggagaacgggatgatgaatacgaacaatactgtgtgcggcgcgaaccatgacggagcgatgagtctggcggccccaggcgattacggtgtggcctgctaccggattccggcccttgccgaggcgcccaacggctggatcctcgcggcgttcgacgcgcggccccataactgccaggatgccccgcaagccaattcgatcgtgcagcgtatctccaaagacggcggccggtcattcgagccgcagcatgttgtggccgccgggcatgatggcgtcgacaaatacgggtattccgatccgtcctatgtggtggaccggcagacgggagaggtgttcctgttcttcgtcaaatcctatgacgccggtttcggaacctcccaggcgggcgtcgatccctctgcgcgtgaggtgcttcaagccgccgtcaccagttccatcgacaatggcgtgacgtggagcgagccgcgcatcatcaccgccgacatcacgaacagcgaatcatggatttcacggttcgcttcttccggtgccggcatccagctcacgtatggcgagcatgcggggcgcctgatccagcagtacaccatcaaggagctcgacggccgctaccgtgcggtatcggtcttctccgacgatcacggtgcaacctggcatgcaggcacccccgtcggcgatcacatggacgagaacaaagtcgtggaactttccgatggccgtgtgatgctgaactcgcgttcctccgatggaaacggttgccgctatgtcgccatctcccgggacggtggcgccacgtatggtccggtgatccgtgaaacgcagctgcccgatcccgagaacaacgcgcagattgcccgtgcgttccccgatgcccccgaggggtcggcgcaggccaaggttctgctgtattcctcctcgtcgccttcggacaggatcgatggtctggtgcgcgtctcgatcgatgacggcaagacctggagtgccggccgacggttcacgacagggccgatggcgtattcggtgatcgccgcattgagccacaaggccggcggcggctatggcctgctgtatgaaggtgataataataacattatgtacacccgtatctcgctcgactggctcaacggccagctgaacgtcgacggaatcggcggttttccgctgtctggtgagggagggtgctga

SEQ ID NO: 68 - DNA, 438 bp, Bifidobacterium longum subsp. infantis
atgctgaagggaataccgccgatcatacagcccgatctgctgaagatactcagcgagatgggccacggcgatgccatagtccttgcggacgcccactttcccgccgaatcggtgggcgtccgatcccacgtgatcaggtatgacggccagcccatcgagccgctgctcgacgcggtgctgcagctgataccgctggaccaatacacggaacacccggtgctgctgatggacaaggttcccggagacaccgtggacaccccgatatgggaccggtaccgtcaggtcatcgacaggcacgagcccggcaagcaagcgggcatcgggatgctggaacggttcgccttctacgaggaggccggcaggtcctattgcatcgtcgccaccggcgaacaatcgcagtatgcgaacatcatcatcagaaaaggcgtcattcgctaa

SEQ ID NO: 69 - DNA, 705 bp, Bifidobacterium longum subsp. infantis
atgtccgtacgaaaaacgtctatcatgtgcactatggggaacccaaatgaaatcagactggcgatagtcgataacgacgacttcgtgctgatgggtttggcagcgttcttgtcgcgtcatctgccgaatgttcggttagcttggaaggcgaataccggaaccgatgctctggaatatgcgacggatcccgcaaatgaagcggacattctgctggttgacatgagtctggaggacatgcccggagacatggtgtgccgggaaatcagaagtcgtaacaggatgttgccgttgctggcggtgacatcgttcagtttaactcgctatgcgcgacgtgctgctgagggtggtgctcaaggcattgtgtcaaaagctgattttccagtactgtgtaaagcggtcaagctcgtcagcgatggtcatactctctgtgttcgagtaggaggggagactattggattcgaggatgtagatgctgcatatcatcgtctggttcgacttcccgtgaatagaatcgaaagattgtcggaacgggaaaaatatgccatggaactatattcacagtcgtataagcccactcagattgcccggatgatggatgtttcggcagggacggtgaaaacctatcttgaccgtgctcagaacaaactccatcttacttccagagccgaactgattgcctattggtggaggcgggaacgatgctga

SEQ ID NO: 70 - DNA, 1722 bp, Bifidobacterium longum subsp. infantis
atgagaaccatcaccagaagcgactatgcgggcatgttcgggccgaccaccggcgatcgggtgcgcttgggggatacgggtctgctgatcgaggtcgagaaggattacgcgcgttatggcgatgaattgaaattcggcggcggaaaatccttccgggacggcatggggcagtcatccgtccagaaggactccgaatcgccggacacggtcatcaccaacgcgctcgtcgtcgattacaccggcatctacaaagcggatatcggcatcaaggatgggaagatcagcgcgatcggcaaggccggcaacccgcagaccatggacggggtgaccccgggtcttgcagtcggcgcgtgcacggaggcaatcgccggtgaagggctcatcctcaccgccggcggcatcgacacccatattcatttcatcgcgccgcagcaagtccgcacggctcttgccggtggcgtcaccaccatggtcggcggcggcaccgggccggctgacggcaccaacgccaccacatgctcgccgggcgcgttccacatcgcccgcatgatcgaagccgccgaagccatgccggtgaacatcgcgtatctgggcaaaggcaacggatcatcgccggaaccattgcgcgagcagattcgtgcgggagcggccggattgaagatccacgaggattggggcgcgaccccggccgtcatcgacacgtgcctcggtgtcgccgacgacatggacgtgcaggtggccatccatacggacaccctgaatgagggaggatgcgtcgaagacaccatcgccgcattcaagggccggaccatccacacctatcacaccgaaggggcgggcggcggccacgcgcccgacatcatccgcgcggccagtttccccaacgtgctcccgagctccacgaacccgaccatgccgttcacccgtaacaccatcgatgaacatctcgacatgatgatggtcacgcatcatcttgaccgcaatgtgccggaagacatcgccttcgccgattcgcgtattcgccccgagaccatcggggccgaggatgtgcttcatgaccttgggctgatctccatgatgagttcggactcgcaggccatgggacgggtcggagaggtcatcacccgtacctggcagacggccgacaagatgaaaaagcaacgcggcccgctccccgaggatgcgcacgacgacaaccgcaacgacaatttccgtgtgaagcggtacgtgtcgaaatacaccatcaatccggccatcacgcacggcatctccgactacgtcggttccgtcgaagtcgggaaaatggccgatctggtgctttggcagccggcgctgttcggcgccaaaccggagatgatcatcaagggaggatcgatcctccatgcgcgtatgggcgacgcgaatgcgtccatacctactcccgagccggtgctgtaccgcgacatgttcggcgcgatgggcaaagccttgggctcctcatgcgccacattcgtctcgcaggccgcccatgacgacgacatcgccggccggctcggtctcgaacggcaggtgcttccggtgcgccactgccgtggcatcggtaagaaggatctgaagttcaatgacaccatcgcggatattcaggtcaaccccgagacgttccaagtgagcgtcgacggcgagccgattcacagcgaaccggtggcggaactgccgttggcgcagcggtacttcctgttctag

SEQ ID NO: 71 - DNA, 963 bp, Bifidobacterium longum subsp. infantis
atgacaagcacatccatcaatccggcgccggtccgcggcgcggggtcggacgacatcccgttcaacccgaagctgaggcagaggaggagcgtgtccgactgggtcgtcgacatcgtgatctggctgttgatcgcgttggtcgtggtggcgatcgtctatccgatctggttcatcgtgatcgcctcggtctcggaccagacgatggtcagccagggcaaggtgttcctgttgcccgcgaagatgaacttcggcggttacgccaaggtgttcaccgattcgaggatctgggtcggctaccgcaacaccatcttctattcggtggcgggcacggccctgaacatgctggtcacgatcccggcggcgttcgcgttgtctcgccgcgagttcaagccgcgccgcgtgatcctgttcctgatgacgttcacgatgttcttctcgggcggcatgatcccgagcttcctgctgtacaagcagctgggtctgctgaactcggtgtgggtgttcatccttccgggcgcggtgagcgtgtggaacctgatcgtggcgcgttcgttcttcgagagctcgatcccggagagcctgcatgacgccgcgcagatcgacggcctggggtatttcgggtatttcctgcggatcgtgctgccgttgagctccgcgatcctggcggtgatgacgttgtattacttcgtgggtcattggaacgacttcttcactggcttggtgtacatccgcgacgcggacaagctgccgttgcagaacgtgttgcgttccatcctgttgtcgaaccagacgaacatcaccggtcagagttcgggtggcatggacgtggtgcagcagcgtgatttcgcgaaccagatcaagtacggcgtgatcatcgtgtcgacgttgccgttgctggtgttgtatccgttcctgcagaagtatttcaacaagggcgtgatgatcggtgccgtgaagggctga

SEQ ID NO: 72 - DNA, 954 bp, Bifidobacterium longum subsp. infantis
atgtctgtggtgaagcagggtgaagtggtggataacgcgttggcggctcgttccgcgccgttgccggtgcggttggggcgtcatttccgtcggtacggcgcgttgtggctgatgacgttgccggcgttggtgttcgtcggtctgttcgcgtacgtgccgatgtacggtcttcgtctcgcgttctatgatttcgatccggtcgagggtctgatgggcggcaggttcgcggggctgcggtatttcgagcagttcttccggtcgggcatgttcgtgaacatcatggtcaacacgttgcggatcagcctgtggacgctggtcatggggttcatcgccccgatcgtgctggcgttgctgatcaaccagatctcgtcgtcgaagatcaaggggttcgtgcagacggtgacgtacatgccgcacttcatctcgacggtcgtgatcgtgtcgatgatcaacatcttcctgtcgccgagcacgggcatgatcgggcgcctgttcccgggcaccgacctgttgggcgagccggggctgttcacgccgatctactggatcagcgaggtgtggcagcacatgggttggaactgcatcatctatctggcggcgttgagttcggtggacctgtccctgtacgaggccgcgaagatcgacggggccggtcgtctgcagctcatccgctacgtcgacatcccggcgatcatgccgacggtgggcatcatgctgatcatgaacatgggttccgtgctcaacgtgggcttcgagaaggtgctgttgatgcagaacccgatgaacctgtccgtgtcggaggtcatcgcgacgtacacgtaccgcatgggtattctgggcaaccagttcagctacacgacggccataggcctgttcaacaccgtggtcaacttcttcttcctggtgctcgccaacttcatttcgaagagggcgtcggacaccagcatcttctag

SEQ ID NO: 73 - DNA, 1395 bp, Bifidobacterium longum subsp. infantis
atgactgatacacatgatttcacgtcattgcccagattgaaccgcgcagagcacggccgcgccgcagcgccggtgcgcatcatccatcttggtatcggcaatttcaccagggcgcatcaagcctggtacacggaacatgcgccggacaccgcgcagtggggcatcgccggtttcaccggacgttctcgccgaatggtcgacaagctggcccctcaggacaacgtctacacgctgatcacctcggcaccggaaggcgattcgttcgaagtcatctcttcgatttcatcgatgcatgagggcggcgacatggcggcactgcacaagtacttcgcggatccgaacgtgtccatcgtcacctccacggttaccgaagctggatacaaaaggaacgcccacggtgatctagatgtcaacgacaccgacgtgtcggaggacctcgccaaactgcgtaacgatgtgaacgtgtccgaacttgccaccgtgcctgcacgcattgtggccgggctggaggcgcgtcgcgcggcaaacgccggagcgatcactatccttccctgcgacaacctcgcaggcaacggagccgcgttccgtcgcgtcgtcgagcaggccattgaggccgtggaccccaccttgctggaatggactcgcgacaacgtggcatgggccacttcgatggtcgaccgcatcacgccggccaccaccgacgccgaactgcagaccgtggcacaggacgagggatggtacgacgtggcgccggtgcgcacggaaccgttcctcgaatgggtcatccgaggcgacttcccgaagggacgtcctgcatgggacaaggccggtgccgttatcacggatgatgtggaaccctacgagcagcgcaaactgtggatgctcaatggttcgcattccactctcgcctatgtcggcccgctgttcggtcatgagtcggtggccgaggccatcgctgacccgcagctgcgggcatgggtgaatgaatggtgggatctggccggatcatacctgtccgtctccagtgacgactaccgtgccaagctgttggagcgattctccaatccgcgcattcaccatcaccttttccaaatcgccaccgatggttcgcaaaagctgccggtacgaatcgtgcctgtcgccaagcacgcgcttgcggacggtcgcgacatcacggctccggcacgtgctgtcgccgcatggatcgtgttcctgcgtcgatttggaaacacggaggccaaggacgtgaatctcgacgaggtgaggaaacttgccgtcaacgatgacgtcaccccagccgtgtcctacctggacaaggatcttggcgccaacgccactttcaccaacaaggtgagcgaactcgtcaacgagctgtcttcggcgacagccgcgtga

SEQ ID NO: 74 - DNA, 1431 bp, Bifidobacterium longum subsp. infantis
atggtatccgcgctcaatgaagatcgcctattcccaacggatccggaacaacgtttaatcgcacgtcgactgtacgaggcaatcaaggatcgtcccatcatctcccctcacggacacgtccctatcgactggttcgcggaagacaagcatttcaagaaccccaccgatctattcatcactcctgatcactacgtgacccgtatcatgcacggacatagtgttcctttcagcgagctgggtgtgggccagaagaacttcaccgaagaacaatctcgcaacgccttccgcctccttggaaagtactggtatgcctatgctggcacgccgatgcgttactggatggaggattcgctgtccaatgtgttcggcatcaacaagccactcaatgaagacaccgcagacagcatctacgatgagctcaacgagctccttgcctccgacgatttcaccacccgcaagcttgtgaagcgtttcaacatcggcttcatctccaccaccgatgaccccaccgatgacctggtcctgcacgataaggtgcgcgccgacgccaatttcccggcgcgtcttgcaccgtgtttccgtccggatcgctaccttgcggtcgatcgtgtggattgggctcagctctgcgaccagctgggtgaatcggctggcgtaaacaccgccacttatgaaggattcgtagaggccatgcgccgtcgtcgtctgttcttcaagcagcatggcgcagtcgccgccgattacggtgtggatccttcgcttgacgaagtcaattggtccggcgacaccacacgcctgtcggatgatgtggcgattcgcttgtacaccaaggctcgtagtggcagcctgacttccgatgaggcacgcaagctgcacgcccatctgctcaacgaccaggcaaagctcgcgcaggacgacggtctggtaatgaccttgcaccccggcgtgatgcgcaaccactaccgcaagcagctcgtcaactacggtccggactgtggagcggattgcccgatgccggctgattgggctcactggctgcgccccatgctcaacgaatacggcgagaatcctgatttccaccttgtggcattcaccatggatgagaccgcatactcccgcgagctcgctccaatggcggcatactacccggcgctgtacatcggagccccgtggtggttcctggatgcgcctgagccgattctgcgctactacgaggatgtcgttccgtacgctggtttcgccaagctgtcgggattcatcgatgacacccgtgcgctgtgctccatccccgcccgccacgacatgaaccgtcgtctgaccgcacgttacatctccggtctggtcgccgaccaccggttgagttatgaggaaggcgagcagatcgccattcgttccgtggacggtcagccttctgacgtgttcaagctctga

SEQ ID NO: 75 - DNA, 1536 bp, Bifidobacterium longum subsp. infantis
atgggagataagaaacaactgcttgtgaacatggtcgcaagtttggtgaatttcacggtttccgttggaatcggtctggtgcttacgccatatatcgtgcgtagcatcggcgctgaagcctatgggttcgtcggattggcgaacacgttcgtgagctatgcgcagctgcttaccatcgcattgaattcggtggccgggcgcttcatcacggtcgcataccatgaaggcgatgattccaaggcgaacgggtattattcgtctacgcttgcggcaaacggcgtcatggtcgccatactggtggtcgtcgcggttccggtggtgacgttccttgacaagctggtgaacatctctccacatcttgtgggagacgtgaaggctctgttcgtcttcatattcctgaatttcatgctttcgaccatcgccacggtgtattccgtcgccacattcgtgaagaacaagctgtatctgagcagtatcgcgaatctcgcgttttccttggtacgtgtggtggccatggtcgcgcttttcgggattctgccgccaaaggtctactacgtgggcttggccgtatgcttggcgacggcggtgatgacgctgatgaatcgctcgtatacgcgcagattacttcccgacatttccttcgacaggaaatcggtatcgtggacgagcattcgtgagatgctttccgccggcgtgtggaacgtggtcacgaaactccagcagattatgatgttcggattgcagttgctggtggcgaacctgatgatcagcccgtatcttatgggtatgctctccatcgcccagaccgttccgaaccagatcagcggactaatgtggactgtgtcgagcctgttttatcctgaacagacgaaatactatgcgcagggcaagcataaggaactcatcgaagacctcaaatcgggcatgaaggtgaccggtttcttcacgaatattatttttgtggcgatgcttgttgcaggatatgatttcatgagtttgtggcagcatggtcaggatacggagctgttgtatcagttgcttacccttaccatgctgggcttgctgatttccggtgtcgctacgacgttgcagaatctgccgctgatcgtcaaccgtttgaagaagtattctatcggatggctcgtctacagtgcgatatccatggtcgtgctgattgcctttatcgaggttcttccgaagtggggcgttttcctcgtggcggcgattccgcctctgtttgagattttggcaaatgtgacattcgtgcccgtttatgcctccagatgcttgggcatcgggaaattcgagttctatcccatctatattcggtacttcgcctccacagcggtggctgccgccgtctgctggggaatcaggcatgtgttcgcgctggtcgccaatggctgggtctcgttgattctgacgtgttgcctgtatgcattggtcacaatgcttttggatgtagtgttgttgctcggaaagaaagaacgttccatgttggtaggtatgctgaagaagaagctacatatcaactag

SEQ ID NO: 76 - DNA, 897 bp, Bifidobacterium longum subsp. infantis
atgaccagcaacgccatgttcgaaggcgtgttctgcccctccatcaccatcatgaacgcggacggaaccatagattacgacaactggggcaagcacctcgaccacctcgtcgacgcaggcgtcgacggcgtgctgctgttcggcagcatcggcgaattctacgccatcgacgtcaagacgaaggcggaagcggctcgcttcgccgtctcgaaggtcgccggacgcatgaaggtgctcgtcggcgtgggagacaccaacctggacaacgtgaaggcgttggcggcggaaagcgaagcggccggagtcgacgcgctgctcgccgtgtccccgtactatttcggcccgtccccggattgcgcgaaacggtacttctcggcggtggccaaggcgacgaccctgcccgtcatcctgtacaacttcccggcccgaacgggcaacgacctcacgcccgagctggtggccgaactcgccggcgagaacccgaacatcgtcggcatcaaagacaccgtcgacaccatcagtcacaccaggaaggtcatcgcggccgtccgcaaggtgaacccgtcgttcagcgtgctgtccggattcgacgaatactacatcgtcaaccggatcagcggcggcaacggcgtgctgagtggtctgaccaacgtggaacccgagacgttcgtcaaactgcaccgcgcatgggaggccggcgaccacgccgcggtcgtcgaagcggccgagcgcgtctcctacctgatgcgcctgtacgacaccgccgacctgttcatcagcgccatcaagggcgcggtcaaggccaagggactacccatcgacacgtccgtccacgagcccgccgtgcagctgaccgacgagcagtatcgcaccatccgcgccatcctggacaagtga

SEQ ID NO: 77 - DNA, 1266 bp, Bifidobacterium longum subsp. infantis
atgacatttcccgaaggattccagtttggtacggccacggcctcgtatcagatcgaaggcggcgccaccgagggcggccgcgcgccatcgatttgggacacgttctcccacacgcccggcaaaaccgtgaacggcgataccggtgatgtggcctgtgattcctaccatttgtggcagaaggacatcgatttgctcgccgatctgggcgtggactcctaccgtctgtctgtggccatgccgcgtgtgatgccgaccgaggacggcccggtcaacgaggagggccttgactactacgagcgcgtggtcgacgcgttgctcgacaagggcatcaagcccacggtcacgctctaccactgggatctgccgcagtacctcggcgatgggaacggttggctcaaccgcgacaccgcctacaaactggccgactatgcgcgcatcgtggctcaccgtctgggcgaccgcgtcgaaacctacaccacgttgaacgagccgtggtgctcctcctacctgagctacggcgccaccgagcacgcacccggtctgggccttggtcccggtgccttcccggccgtgcatcatctgaatctggctcatggtctgatggcccaggcggtgcgtagcgaggtcggcgacaggtcgcagctgtcggtcacgctgaacctgcagttcaaccgtggcgacgccgacgccgtgcaccgtctcgacctgatcggcaaccgcgtgtggctcgacccgatgctgcgcggctactacccgaacgagctgttcgccatcaccaagggcatctgcgattgggagttcgtcaaggacggcgacctcgaacagatccatcagccgctggacgtgctcggcatcaactactattcctccggcttggtcacgatgagcggacgcccgcagttcccgcagtccacgggcccgagcaccgcgcctggtgccagcgacgtcgactggctgccgacgcccggcgagcacaccgacatgggctggaacatcgatccgaagggcctatatgacctgctgatgcgcgtgcataacgattatccagagattccgctgatggtcaccgaaaacggcatcgcggtcgagggcggcgaccgggtcgtcaccgaggcggatggcaccaaggccgtgcacgaccccaagcgcatcgactacctgaagcggcacttcgaggccgcactcaaggctatcgaggacggcgtggatttgcgcggctacttcgtgtggtccatcgcaccagctgccaagcagtccggctactga

SEQ ID NO: 78 - DNA, 165 bp, Bifidobacterium longum subsp. infantis
atgacattgccgccgcaatcgcgaaaagacgatgactgcggcggcatcctcaacatcactccaacagaaagcatgacccacggcaccacgcgtcaggcgctcagtctcatcaccaatcccatcatcaccatcgtgctttttgcgctcgccgtgttcgcctgctga

(19) United States
(12) Patent Application Publication
(10) Pub. No.: US 2025/0268927 A1
(43) Pub. Date: Aug. 28, 2025

(54) SYNBIOTIC TREATMENT REGIMENS

(71) Applicant: PROLACTA BIOSCIENCE, INC., Duarte, CA (US)

(72) Inventors: Gregory MCKENZIE, Duarte, CA (US); Scott ELSTER, Duarte, CA (US); Julie E. BUTTON, Duarte, CA (US)

(21) Appl. No.: 18/261,012

(22) PCT Filed: Jan. 12, 2022
(86) PCT No.: PCT/US2022/012120

§ 371 Date: Jul. 11, 2023

Related U.S. Application Data
(60) Provisional application No. 63/136,469, filed on Jan. 12, 2021.
(60) Provisional application No. 63/165,549, filed on Mar. 24, 2021.

(51) Int. Cl. A61K 31/702; A61K 35/20; A61K 35/747; A61P 43/00

(52) U.S. Cl. CPC A61K 31/702; A61K 35/20; A61K 35/747; A61P 43/00

(57) ABSTRACT

Provided herein are compositions, methods, strategies, kits, and articles of manufacture that are useful, inter alia, in the treatment or prevention of diseases, disorders, or conditions that may be associated with inflammation, infection, allergy, immune dysfunction, or dysbiosis of the intestinal microbiome, such as graft versus host disease (GVHD). In some aspects, the invention provides a synergistic combination of prebiotics that are synthetic or derived from human milk with a probiotic strain of bacterium, such as a strain capable of internalizing and consuming the prebiotic, e.g., Bifidobacterium longum subsp. infantis.

### CROSS-REFERENCE TO RELATED APPLICATIONS

This application claims priority from U.S. provisional application No. 63/136,469 filed Jan. 12, 2021, entitled “SYNBIOTIC TREATMENT REGIMENS” and U.S. provisional application No. 63/165,549 filed Mar. 24, 2021, entitled SYNBIOTIC TREATMENT REGIMENS”; the contents of both incorporated by reference in their entirety.

### INCORPORATION BY REFERENCE OF SEQUENCE LISTING

The present application is being filed along with a Sequence Listing in electronic format. The Sequence Listing is provided as a file entitled PROL_042_03WO_SeqList_ST25, created Jan. 5, 2022 which is 154 kilobytes in size. The information in the electronic format of the Sequence Listing is incorporated by reference in its entirety.

### FIELD OF THE INVENTION

Provided herein are compositions, methods, strategies, kits, and articles of manufacture that are useful, inter alia, in the treatment or prevention of diseases, disorders, or conditions that may be associated with inflammation, infection, allergy, immune dysfunction, or dysbiosis of the intestinal microbiome, such as graft versus host disease (GVHD). In some aspects, the invention provides a synergistic combination of prebiotics that are synthetic or derived from human milk with a probiotic strain of bacterium, such as a strain capable of internalizing and consuming the prebiotic, e.g., Bifidobacterium longum subsp. infantis.

### BACKGROUND OF THE INVENTION

The microbiome is proposed to be a key modulator of human health, such as to the extent that it has been proposed to be an ‘essential organ’ of the human body. For most individuals, microbial colonies found on or in the body, such as in the gut, are normally benign or beneficial. These beneficial and appropriately sized microbial colonies carry out a series of helpful and necessary functions, such as aiding in digestion or preventing growth of pathogenic microbes. Changes to the microbiome composition, such as from the presence or expansion of pathogenic microorganisms or a loss of the diversity of the microflora, may result in a state of dysbiosis. While microbiome dysbiosis has been described in various diseases, safely promoting a ‘healthy’ microbiome has been difficult, particularly in subjects who may be vulnerable or immunocompromised. Furthermore, microbiomes may vary in healthy individuals, adding to confusion over how a “healthy” microbiome may be defined, let alone promoted or developed.

What is needed in the art are compositions and methods for safely treating or ameliorating dysbiosis of the microbiome, as well as for treating or preventing disorders or diseases involving inflammation, infection, allergy, or immune dysfunction that may be associated with dysbiosis.

### SUMMARY OF THE INVENTION

Provided herein are compositions, kits, articles of manufacture, and methods of use thereof, that contain prebiotics, e.g., non-digestible carbohydrates such as human milk oligosaccharides, and one or more probiotic strains of bacteria capable of consuming the prebiotics. In some aspects, the prebiotics may include one or both of a concentrated human milk permeate composition containing human milk oligosaccharides and one or more synthetic human milk oligosaccharides. The prebiotics, e.g., the concentrated human milk permeate composition and/or the synthetic human milk oligosaccharides, may be administered to facilitate, augment, or maintain the colonization, engraftment, or expansion of the probiotic. The provided compositions, kits, and articles of manufacture are particularly useful for treating or preventing diseases or conditions, such as those involving inflammation, immune disorders, allergy, or dysbiosis of the intestinal microbiome. In some aspects, the provided compositions, kits, and articles of manufacture are useful for treating, preventing, and/or reducing the risk or likelihood of graft versus host disease (GVHD).

Provided herein is a method for treating or preventing a disease, disorder, or condition associated with one or more of dysbiosis of the intestinal microbiome, inflammation, infection, allergy, or immune dysfunction in a subject in need thereof, the method comprising administering to the subject (i) a concentrated human milk permeate composition comprising human milk oligosaccharides; (ii) at least one probiotic strain of bacterium capable of consuming human milk oligosaccharides; and (iii) one or more synthetic human milk oligosaccharides; wherein the one or more synthetic human milk oligosaccharides are administered at least once on a day that occurs after a day wherein one or both of the concentrated human milk permeate composition or the probiotic strain are administered.

Also provided herein is a method for maintaining engraftment of at least one probiotic strain of bacterium in a subject in need thereof to treat or prevent a disease, disorder, or condition associated with one or more of dysbiosis of the intestinal microbiome, inflammation, infection, allergy, or immune dysfunction in the subject, the method comprising administering to the subject one or more synthetic human milk oligosaccharides, wherein the subject has previously been administered the probiotic strain of bacterium and a concentrated human milk permeate composition comprising human milk oligosaccharides, and wherein the probiotic strain of bacterium is capable of consuming human milk oligosaccharides.

In some embodiments, the probiotic strain is capable of internalizing human milk oligosaccharides. In some embodiments, the probiotic strain comprises a bacterial strain of the genus Bifidobacterium. In some embodiments, the probiotic strain comprises a strain of B. longum subsp. infantis, B. longum subsp. longum, B. breve, or B. bifidum. In some embodiments, the probiotic strain comprises B. longum subsp. infantis.

In some embodiments, the concentrated human milk permeate composition comprises at least 10, at least 25, at least 50, or at least 100 human milk oligosaccharides. In some embodiments, the concentrated human milk permeate composition comprises at least 10 human milk oligosaccharides. In some embodiments, the concentrated human milk permeate composition comprises 2′-fucosyllactose, 3-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose. In some embodiments, the concentrated human milk permeate composition is obtained from human milk permeate that results from the ultrafiltration of human skim milk, wherein the human skim milk is obtained by removing cream from pooled human milk, and wherein the pooled human milk is pooled from the milk of multiple human milk donors. In some embodiments, the pooled human milk is pooled from the milk of at least 25, 50, or 100 human milk donors.

In some embodiments, the one or more synthetic human milk oligosaccharides comprises one or more of 2′-fucosyllactose, 3-fucosyllactose, 3′-sialyllactose, 6′-sialyllactose, lacto-N-tetraose, lacto-N-neotetraose, lacto-N-fucopentaose I, lacto-N-fucopentaose II, lacto-N-fucopentaose III, sialyllacto-N-tetraose a, sialyllacto-N-tetraose b, sialyllacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-lacto-N-hexaose a, difucosyl-lacto-N-hexaose b, lactodifucotetraose, 6′galactosyllactose, 3′galactosyllactose, 3-sialyl-3-fucosyllactose, sialylfucosyllacto-N-tetraose, sialyllacto-N-fucopentaose V, disialyllacto-n-fucopentaose II, disialyllacto-n-fucopentaose V, lacto-N-neo-difucohexaose II, 3-fucosyl-sialylacto-N-tetraose c, para-lacto-N-neohexose, lacto-N-octaose, lacto-N-neooctaose, lacto-N-neohexaose, lacto-N-fucopentaose V, iso-lacto-N-octaose, para-lacto-N-octaose, lacto-decaose, or sialyllacto-N-fucopentaose I. In some embodiments, the one or more synthetic human milk oligosaccharides comprises one or more of 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, or disialyllacto-N-tetraose.

In some embodiments, the one or more synthetic human milk oligosaccharides comprises one or more of 2′-fucosyllactose, 3-fucosyllactose, lacto-N-tetraose, or lacto-N-neotetraose. In particular embodiments, the one or more synthetic human milk oligosaccharides comprises two or more of 2′-fucosyllactose, 3-fucosyllactose, lacto-N-tetraose, or lacto-N-neotetraose. In some embodiments, the one or more synthetic human milk oligosaccharides comprises at least three of 2′-fucosyllactose, 3-fucosyllactose, lacto-N-tetraose, or lacto-N-neotetraose. In certain embodiments, the one or more synthetic human milk oligosaccharides comprises 2′-fucosyllactose, 3-fucosyllactose, lacto-N-tetraose, and lacto-N-neotetraose.

In some embodiments, the disease, disorder, or condition comprises one or more of obesity, type II diabetes, a chronic inflammatory disease, an autoimmune disease, an infection, an infectious disease domination, bowel resection, or a condition associated with chronic diarrhea. In some embodiments, the disease, disorder, or condition comprises one or more of irritable bowel syndrome (IBS), inflammatory bowel disease (IBD), short bowel syndrome (SBS), celiac disease, small intestinal bacterial overgrowth (SIBO), gastroenteritis, leaky gut syndrome, pouchitis, or gastric lymphoma.

In some embodiments, the disease, condition, or disorder is graft versus host disease. In some embodiments, the subject has received or will receive an allogenic hematopoietic stem cell transplant.

In some embodiments, the disease, condition, or disorder is associated with an infection. In some embodiments, the infection comprises a bacterial infection or gut domination. In some embodiments, the bacterial infection or gut domination comprises an infection or gut domination by one or more species, subspecies, or strains of Aeromonas, Bacillus, Bordetella, Borrelia, Brucella, Burkholderia, Campylobacter, Chlamydia, Chlamydophila, Citrobacter, Clostridium, Corynebacterium, Coxiella, Ehrlichia, Enterobacter, Enterobacteriaceae, Enterococcus, Escherichia, Francisella, Haemophilus, Helicobacter, Klebsiella, Legionella, Leptospira, Listeria, Morganella, Mycobacterium, Mycoplasma, Neisseria, Orientia, Plesiomonas, Proteus, Pseudomonas, Rickettsia, Salmonella, Shigella, Staphylococcus, Streptococcus, Treponema, Vibrio, or Yersinia, optionally one or more of Aeromonas hydrophila, Bacillus cereus, Campylobacter fetus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enteropathogenic E. coli, enterotoxigenic Escherichia coli, Escherichia coli 0157:H7, Helicobacter pylori, Klebsiellia pneumonia, Lysteria monocytogenes, Salmonella paratyphi, Salmonella typhi, Staphylococcus aureus, Vibrio cholerae, Vibrio parahaemolyticus, Vibrio vulnificus, or Yersinia enterocolitica.

In some embodiments, the bacterial infection or gut domination comprises an infection or gut domination by one or more of Citrobacter freundii, Citrobacter koseri, Enterobacter aerogenes, Enterobacter cloacae, Enterococcus faecalis, Enterococcus faecium, Escherichia coli, Klebsiella oxytoca, Klebsiella pneumoniae, Lactobacillus acidophilus, Morganella morganii, Proteus mirabilis, Serratia marcescens, Staphylococcus aureus, Staphylococcus epidermidis, Streptococcus anginosus, Streptococcus australis, Streptococcus constellatus, Streptococcus cristatus, Streptococcus gordonii, Streptococcus infantis, Streptococcus intermedius, Streptococcus mitis, Streptococcus mutans, Streptococcus oligofermentans, Streptococcus oralis, Streptococcus parasanguinis, Streptococcus peroris, Streptococcus pneumoniae, Streptococcus pseudopneumoniae, Streptococcus salivarius, Streptococcus sanguinis, Streptococcus sobrinus, Streptococcus tigurinus, or Streptococcus vestibularis.

In some embodiments, the bacterial infection or gut domination comprises an infection or gut domination by drug-resistant bacteria. In some embodiments, the drug-resistant bacteria comprise one or more of antibiotic-resistant bacterium (ARB), Antibiotic-resistant Proteobacteria, Carbapenem-resistant Enterobacteriaceae (CRE), Extended Spectrum Beta-Lactamase producing Enterobacteriaceae (ESBL-E), fluoroquinolone-resistant Enterobacteriaceae, extended spectrum beta-lactam resistant Enterococci (ESBL), vancomycin-resistant Enterococci (VRE), multi-drug resistant E. coli, or multi-drug resistant Klebsiella. In some embodiments, the subject has undergone or will undergo an ileal pouch-anal anastomosis (IPAA) surgery, and wherein the disease, condition, or disorder comprises pouchitis.

In some embodiments, the method comprises two or more treatment phases, wherein the two or more treatment phases comprise a first probiotic treatment phase and one or more subsequent treatment phases; wherein the first probiotic treatment phase comprises administering to the subject the at least one probiotic strain and the concentrated human milk permeate composition; and wherein the one or more subsequent treatment phases comprises at least one synthetic prebiotic treatment phase comprising administering to the subject the one or more synthetic human milk oligosaccharides.

In some embodiments, the first probiotic treatment phase comprises administering to the subject the at least one probiotic strain and the concentrated human milk permeate composition at least once every other day for the duration of the treatment phase. In some embodiments, the first probiotic treatment phase comprises administering to the subject the at least one probiotic strain and the concentrated human milk permeate composition at least once daily for the duration of the treatment phase. In some embodiments, the duration of the first probiotic treatment phase is at least 1 day, optionally from about 1 day to about 14 days, about 3 days to about 10 days, or about 7 days. In some embodiments, the synthetic prebiotic treatment phase comprises administering to the subject the one or more synthetic human milk oligosaccharides at least once daily for the duration of the treatment phase. In some embodiments, the duration of the synthetic prebiotic treatment is at least 1 day, at least 7 days, at least 14 days, or at least 30 days.

In some embodiments, the one or more subsequent treatment phases comprises a permeate treatment phase, wherein the permeate treatment phase is subsequent to the first probiotic treatment phase and prior to the synthetic prebiotic treatment phase, wherein the permeate treatment phase comprises administering to the subject the concentrated human milk permeate composition at least once every other day for the duration of the treatment phase. In some embodiments, the duration of the permeate treatment phase is at least 1 day, optionally from about 1 day to about 14 days, about 3 days to about 10 days, or about 7 days.

In some embodiments, the method comprises a first, a second, and a third treatment phase: wherein the first treatment phase comprises administering to the subject the at least one probiotic strain and the concentrated human milk permeate composition at least once every other day for the duration of the first treatment phase; wherein the second treatment phase comprises administering to the subject the concentrated human milk permeate composition at least once every other day for the duration of the second treatment phase; and wherein the third treatment phase comprises administering to the subject the one or more synthetic human milk oligosaccharides at least once daily for the duration of the third treatment phase; wherein the duration of each of the first, second, and third treatment phases is at least 1 day, optionally from about 1 day to about 14 days, about 3 days to about 10 days, or about 7 days.

In some embodiments, the at least one subsequent treatment phase begins immediately after the first probiotic treatment phase. In some embodiments, the at least one probiotic strain is not administered to the subject during at least one subsequent treatment phase. In some embodiments, the concentrated human milk permeate composition is not administered to the subject during at least one subsequent treatment phase.

In some embodiments, the method comprises a first treatment phase, a second treatment phase, and a third treatment phase; wherein the first treatment phase comprises administering to the subject the at least one probiotic strain and the concentrated human milk permeate composition at least once daily; wherein the second phase comprises administering to the subject the concentrated human milk permeate composition at once least daily; and wherein the third treatment phase comprises administering to the subject one or more synthetic human milk oligosaccharides at least once daily.

In some embodiments, the at least one probiotic strain is administered in an amount of at least 5×10⁶ colony forming units (CFU) per day. In some embodiments, the probiotic strain is administered in an amount of at least 8×10⁷ colony forming units (CFU) per day. In some embodiments, the concentrated human milk permeate composition is administered in an amount of at least 500 mg of total human milk oligosaccharides per day. In some embodiments, the concentrated human milk permeate composition is administered in an amount of between 0.5 g and 25 g, 1 g and 5 g, 2 g and 3 g, 3 g and 6 g, 4 g and 5 g, 5 g and 10 g, 8 g and 10 g, 10 g and 20 g, 15 g and 25 g, 15 g and 20 g, or 17 g and 19 g of total human milk oligosaccharides per day. In some embodiments, the one or more synthetic human milk oligosaccharides is administered in an amount of at least 500 mg of total human milk oligosaccharides per day. In some embodiments, the prebiotic mixture is administered in an amount of between 0.5 g and 25 g, 1 g and 5 g, 2 g and 3 g, 3 g and 6 g, 4 g and 5 g, 5 g and 10 g, 8 g and 10 g, 10 g and 20 g, 15 g and 25 g, 15 g and 20 g, or 17 g and 19 g of total human milk oligosaccharides per day.

Also provided herein is a method for treating or preventing a disease, disorder, or condition associated with one or more of dysbiosis of the intestinal microbiome, inflammation, infection, allergy, or immune dysfunction in a subject in need thereof, the method comprising administering to the subject (i) at least one probiotic strain of bacterium capable of consuming human milk oligosaccharides; and (ii) one or more of 2′-fucosyllactose, 3-fucosyllactose, Lacto-N-tetraose, or Lacto-N-neotetraose. In some embodiments, the subject will receive or has received a allogenic hematopoietic stem cell transplant, and the disease, disorder, or condition is graft versus host disease.

Additionally provided is method for maintaining engraftment of at least one probiotic strain of bacterium in a subject in need thereof to treat or prevent a disease, disorder, or condition associated with one or more of dysbiosis of the intestinal microbiome, inflammation, infection, allergy, or immune dysfunction in a subject in need thereof, the method comprising administering to the subject one or more synthetic human milk oligosaccharides comprising at least one of 2′-fucosyllactose, 3-fucosyllactose, Lacto-N-tetraose, or Lacto-N-neotetraose; wherein the subject has previously been administered the probiotic strain and a concentrated human milk permeate composition comprising human milk oligosaccharides, and wherein the probiotic strain is capable of consuming human milk oligosaccharides, optionally wherein the probiotic strain is B. longum subsp. infantis.

Also provided is a method for maintaining engraftment of at least one probiotic strain of bacterium in a subject in need thereof to treat or prevent a disease, disorder, or condition associated with one or more of dysbiosis of the intestinal microbiome, inflammation, infection, allergy, or immune dysfunction in a subject in need thereof, the method comprising administering to the subject one or more synthetic human milk oligosaccharides, wherein the one or more synthetic human milk oligosaccharides comprise one or more of 2′-fucosyllactose, 3-fucosyllactose, Lacto-N-tetraose, or Lacto-N-neotetraose, wherein the subject has previously been administered the at least one probiotic strain and a concentrated human milk permeate composition comprising human milk oligosaccharides, and wherein the probiotic strain of bacterium is capable of consuming human milk oligosaccharides, optionally wherein the probiotic strain is B. longum subsp. infantis.

In some embodiments, the condition, disease, or disorder is an inflammatory disease. In certain embodiments, the inflammatory disease is one or more of inflammatory bowel disease, multiple sclerosis, rheumatoid arthritis, asthma, or a food allergy. In particular embodiments, the subject is in an intensive care unit and/or is a geriatric patient. In some embodiments, the method reduces risk of an infection in the subject. In certain embodiments, the infection is a bacterial infection or gut domination, optionally associated with a drug-resistant or an antibiotic-resistant bacterium.

Also provided herein is a method of treating or preventing hyperammonemia, comprising administering to a subject in need thereof one or more of 2′-fucosyllactose, 3-fucosyllactose, Lacto-N-tetraose, or Lacto-N-neotetraose, and B. longum subsp. infantis.

Provided herein is a method for maintaining engraftment of a probiotic strain of B. longum subsp. infantis in a subject in need thereof to treat or prevent a disease, disorder, or condition in the subject, the method comprising administering to the subject one or more synthetic human milk oligosaccharides, wherein the subject has previously been administered B. longum subsp. infantis and a concentrated human milk permeate composition comprising human milk oligosaccharides.

In some embodiments, the concentrated human milk permeate composition was obtained from human milk permeate that results from ultrafiltration of human skim milk, wherein the human skim milk is obtained by removing cream from pooled human milk, and wherein the pooled human milk is pooled from the milk of at least 50, 100, or 150 human milk donors; and wherein the concentrated human milk permeate composition comprises at least 10, at least 25, at least 50, or at least 100 human milk oligosaccharides.

In some embodiments, the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose. In some embodiments the one or more synthetic human milk oligosaccharides comprise one or more of 2′-fucosyllactose, 3-fucosyllactose, lacto-N-tetraose, or lacto-N-neotetraose. In some embodiments, the one or more synthetic human milk oligosaccharides comprise two or more of 2′-fucosyllactose, 3-fucosyllactose, lacto-N-tetraose, or lacto-N-neotetraose, optionally 2′-fucosyllactose and lacto-N-tetraose.

In some embodiments, the one or more synthetic human milk oligosaccharides are administered at least once every other day for at least 3, 5, 7, 10, 14, 21, or 28 days. In some embodiments, the one or more synthetic human milk oligosaccharides are administered at least once daily for at least 3, 5, 7, 10, 14, 21, or 28 days. In some embodiments, the one or more synthetic human milk oligosaccharides are administered in an amount of at least 2 g, 5 g, 10 g, 15 g, 20 g, 22 g, or 25 g of total human milk oligosaccharides per day. In some embodiments, the one or more synthetic human milk oligosaccharides are administered are an amount of from 10 g to 25 g of total human milk oligosaccharides per day.

In some embodiments, the B. longum subsp. infantis was previously administered to the subject in an amount of at least 5×10⁶ colony forming units (CFU) per day for at least 3 days. In some embodiments, the B. longum subsp. infantis was previously administered to the subject in an amount of at least 1×10⁸ colony forming units (CFU) per day for at least 7 days, 9 days, or 14 days. In some embodiments, the concentrated human milk permeate composition comprises at least 10, at least 25, at least 50, or at least 100 human milk oligosaccharides, and the human milk oligosaccharides comprise 2′-fucosyllactose, 3-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose.

In some embodiments, the concentrated human milk permeate composition was previously administered in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day for at least 3 days. In some embodiments, the concentrated human milk permeate composition was previously administered in an amount from 10 g to 25 g of total human milk oligosaccharides for at least 7, 9, or 14 days. In some embodiments, the B. longum subsp. infantis and the concentrated human milk permeate composition were previously administered to the subject on the same day for at least 3 days, 5 days, 7 days, 9 days, or 14 days.

Particular embodiments provide a method for maintaining engraftment of a probiotic strain of B. longum subsp. infantis in a subject in need thereof to treat or prevent a disease, disorder, or condition associated with one or more of dysbiosis of the intestinal microbiome, inflammation, infection, allergy, or immune dysfunction in the subject, the method comprising administering to the subject one or more synthetic human milk oligosaccharides in an amount of from 10 g to 25 g of total human milk oligosaccharides per day for at least 7 days, wherein the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose; wherein the subject has previously been administered the B. longum subsp. infantis and a concentrated human milk permeate composition comprising human milk oligosaccharides; wherein the subject was previously administered at least 1×10⁸ colony forming units (CFU) per day of the B. longum subsp. infantis for at least 7 days; wherein the subject was previously administered the concentrated human milk permeate composition in an amount of at least 10 g of total human milk oligosaccharides per day for at least 7 days; and wherein the B. longum subsp. infantis and the concentrated human milk permeate composition were previously administered to the subject on the same day for at least 3 days, 5 days, or 7 days. Certain embodiments further comprise administering at least one dose of the concentrated human milk permeate after at least one dose of the one or more synthetic human milk oligosaccharides have been administered. In some embodiments, the at least once of dose of the concentrated human milk permeate composition is administered at least once between doses of the one or more synthetic human milk oligosaccharides.

Also provided herein is a method for treating or preventing a disease, disorder, or condition in a subject in need thereof, the method comprising administering to the subject (i) a concentrated human milk permeate composition comprising human milk oligosaccharides; (ii) at least one probiotic strain of B. longum subsp. infantis; and (iii) one or more synthetic human milk oligosaccharides; wherein the one or more synthetic human milk oligosaccharides are administered at least once on a day that occurs after a day wherein B. longum subsp. infantis is administered; and wherein the one or more synthetic human milk oligosaccharides are administered at least once on a day that the concentrated human milk permeate composition is not administered.

In some embodiments the concentrated human milk permeate composition is obtained from human milk permeate that results from ultrafiltration of human skim milk, wherein the human skim milk is obtained by removing cream from pooled human milk, and wherein the pooled human milk is pooled from the milk of at least 50, 100, or 150 human milk donors; and wherein the concentrated human milk permeate composition comprises at least 10, at least 25, at least 50, or at least 100 human milk oligosaccharides.

In some embodiments, the concentrated human milk permeate composition is administered in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day for at least 3 days. In particular embodiments, the concentrated human milk permeate composition is administered in an amount from 10 g to 25 g of total human milk oligosaccharides per day for at least 7, 9, or 14 days. In some embodiments, the B. longum subsp. infantis is administered to the subject in an amount of at least 5×10⁶ colony forming units (CFU) per day for at least 3 days. In some embodiments, the B. longum subsp. infantis is administered to the subject in an amount of at least 1×10⁸ colony forming units (CFU) per day for at least 7 days, 9 days, or 14 days. In some embodiments, the B. longum subsp. infantis and the concentrated human milk permeate composition are administered to the subject on the same day for at least 3 days, 5 days, 7 days, 9 days, or 14 days.

In some embodiments, the one or more synthetic human milk oligosaccharides comprises one or more of 2′-fucosyllactose, 3-fucosyllactose, 3′-sialyllactose, 6′-sialyllactose, lacto-N-tetraose, lacto-N-neotetraose, or difucosyllactose; optionally one or two or more of 2′-fucosyllactose, 3-fucosyllactose, lacto-N-tetraose, or lacto-N-neotetraose. In some embodiments, the one or more synthetic human milk oligosaccharides are administered at least once every other day or at least once daily for at least 3, 5, 7, 10, 14, 21, or 28 days. In some embodiments, the one or more synthetic human milk oligosaccharides are administered in an amount of at least 2 g, 5 g, 10 g, 15 g, 20 g, 22 g, or 25 g of total human milk oligosaccharides per day, optionally in an amount of from 10 g to 25 g of total human milk oligosaccharides per day.

In addition, provided herein method for treating or preventing a disease, disorder, or condition associated with one or more of dysbiosis of the intestinal microbiome, inflammation, infection, allergy, or immune dysfunction in a subject in need thereof comprising administering a probiotic strain of B. longum subsp. infantis, wherein the method comprises two or more treatment phases comprising at least a colonization phase and at least one subsequent maintenance phase; wherein the colonization phase comprises administering to the subject (i) the B. longum subsp. infantis and (ii) a concentrated human milk permeate composition comprising human milk oligosaccharides; and wherein the at least one maintenance phase comprises administering to the subject one or more synthetic human milk oligosaccharides, wherein the B. longum subsp. infantis can be detected within the subject's intestinal microbiome throughout the duration of the at least one maintenance phase.

Also provided is a method for treating or preventing graft versus host disease in a subject in need thereof comprising administering a probiotic strain of B. longum subsp. infantis, wherein the method comprises two or more treatment phases comprising at least a colonization phase and at least one subsequent maintenance phase; wherein the colonization phase comprises administering to the subject (i) B. longum subsp. infantis and (ii) a concentrated human milk permeate composition comprising human milk oligosaccharide; and wherein the at least one maintenance phase comprises administering to the subject one or more synthetic human milk oligosaccharides, wherein the B. longum subsp. infantis can be detected within the subject's intestinal microbiome throughout the duration of the at least one maintenance phase.

In some embodiments, the subject receives an allogenic hematopoietic stem cell transplant, and wherein the colonization phase takes place beginning at least 7, 14, or 21 days prior to the allogenic hematopoietic stem cell transplant and lasts at least until 7, 14, 21, 28, 35 days after the allogenic hematopoietic stem cell transplant. In some embodiments, the subject receives treatment with antibiotics beginning at least 5 days prior to the allogenic hematopoietic stem cell transplant lasting until at least 5 days after the allogenic hematopoietic stem cell transplant, optionally wherein the antibiotics comprises one or more of a fourth-generation cephalosporins, a glycopeptide, a piperacillin-tazobactam, a carbapenem, an aminoglycoside, or a quinolone; and wherein the colonization phase lasts until at least 10 days after the end of the treatment with antibiotics. In some embodiments, the concentrated human milk permeate composition is obtained from human milk permeate that results from ultrafiltration of human skim milk, wherein the human skim milk is obtained by removing cream from pooled human milk, and wherein the pooled human milk is pooled from the milk of multiple human milk donors, wherein the pooled human milk is pooled from the milk of at least 50, 100, or 150 human milk donors; and wherein the concentrated human milk permeate composition comprises at least 10, at least 25, at least 50, or at least 100 human milk oligosaccharides.

In some embodiments, the colonization phase comprises a duration of at least 3 days, 5 days, 7 days, 9 days, or 14 days. In some embodiments, the B. longum subsp. infantis is administered to the subject at least once every other day or at least once daily during the colonization phase. In some embodiments, the B. longum subsp. infantis is administered in an amount of at least 5×10⁶ colony forming units (CFU) per day during the colonization phase In some embodiments, B. longum subsp. infantis is administered to the subject in an amount of at least 1×10⁸ colony forming units (CFU) per day during the colonization phase. In some embodiments, the concentrated human milk permeate composition is administered to the subject at least three times, five times, seven times, nine times, ten times or fourteen times during the colonization phase. In some embodiments, the concentrated human milk permeate composition is administered at least once every two days or at least once daily during the colonization phase. In some embodiments, the B. longum subsp. infantis and the concentrated human milk permeate composition are administered on the same day at least once, three times, five times, seven times, nine times, or fourteen times during the colonization phase.

In some embodiments, the concentrated human milk permeate composition is administered to the subject in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day. In some embodiments, the B. longum subsp. infantis (i) is detectable within the subject's intestinal microbiome at the end of the colonization phase; and/or (ii) is detectable at a greater amount and/or as a greater portion of the total microbiota of the subject's intestinal microbiome at the end of the colonization phase than what is detectable prior to and/or on the first day of the colonization phase. In some embodiments, the one or more synthetic human milk oligosaccharides are administered at least once during the colonization phase.

In some embodiments, the maintenance phase comprises a duration of at least 3 days, 5 days, 7 days, 9 days, 14 days, 21 days, or 28 days or 3 months. In some embodiments, the maintenance phase comprises administering to the subject the one or more synthetic human milk oligosaccharides at least once every two days or at least once daily. In some embodiments, the one or more synthetic human milk oligosaccharides are administered in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day. In some embodiments, the one or more synthetic human milk oligosaccharides are administered in an amount from 10 g to 25 g of total human milk oligosaccharides. In some embodiments, the one or more synthetic human milk oligosaccharides comprises one or more of 2′-fucosyllactose, 3-fucosyllactose, 3′-sialyllactose, 6′-sialyllactose, lacto-N-tetraose, lacto-N-neotetraose, or difucosyllactose; optionally one or two or more of 2′-fucosyllactose, 3-fucosyllactose, lacto-N-tetraose, or lacto-N-neotetraose. In some embodiments, the concentrated human milk permeate composition is administered at least once, three times, five times, seven times, nine times, ten times, or fourteen times during the maintenance phase. In some embodiments, the B. longum subsp. infantis is administered at least once, three times, five times, seven times, nine times, ten times, or fourteen times during the maintenance phase.

In some embodiments, the colonization phase and the maintenance phase are repeated in two or more cycles, optionally wherein the cycles are repeated after a rest period comprising at least one, three, seven, or fourteen days.

In some embodiments, the bacterial infection or gut domination comprises an infection or gut domination by one or more species, subspecies, or strains of Aeromonas, Bacillus, Bordetella, Borrelia, Brucella, Burkholderia, Campylobacter, Chlamydia, Chlamydophila, Citrobacter, Clostridium, Corynebacterium, Coxiella, Ehrlichia, Enterobacter, Enterobacteriaceae, Enterococcus, Escherichia, Francisella, Haemophilus, Helicobacter, Klebsiella, Legionella, Leptospira, Listeria, Morganella, Mycobacterium, Mycoplasma, Neisseria, Orientia, Plesiomonas, Proteus, Pseudomonas, Rickettsia, Salmonella, Shigella, Staphylococcus, Streptococcus, Treponema, Vibrio, or Yersinia, optionally one or more of Aeromonas hydrophila, Bacillus cereus, Campylobacter fetus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enteropathogenic E. coli, enterotoxigenic Escherichia coli, Escherichia coli 0157:H7, Helicobacter pylori, Klebsiella pneumoniae, Listeria monocylogenes, Salmonella paratyphi, Salmonella typhi, Staphylococcus aureus, Vibrio cholerae, Vibrio parahaemolyticus, Vibrio vulnficus, or Yersinia enterocolitica.

Provided herein is a method for maintaining engraftment of a probiotic strain of B. longum subsp. infantis in a subject in need thereof to treat or prevent a disease, disorder, or condition in the subject, the method comprising administering to the subject one or more synthetic human milk oligosaccharides, wherein the subject has previously been administered B. longum subsp. infantis and a concentrated human milk permeate composition comprising human milk oligosaccharides.

In some embodiments, the concentrated human milk permeate composition was obtained from human milk permeate that results from ultrafiltration of human skim milk, wherein the human skim milk is obtained by removing cream from pooled human milk, and wherein the pooled human milk is pooled from the milk of at least 50, 100, or 150 human milk donors; and wherein the concentrated human milk permeate composition comprises at least 10, at least 25, at least 50, or at least 100 human milk oligosaccharides.

In certain embodiments, the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose. In particular embodiments the one or more synthetic human milk oligosaccharides comprise one or more of 2′-fucosyllactose, 3-fucosyllactose, lacto-N-tetraose, or lacto-N-neotetraose. In some embodiments, the one or more synthetic human milk oligosaccharides comprise two or more of 2′-fucosyllactose, 3-fucosyllactose, lacto-N-tetraose, or lacto-N-neotetraose, optionally 2′-fucosyllactose and lacto-N-tetraose.

In certain embodiments, the one or more synthetic human milk oligosaccharides are administered at least once every other day for at least 3, 5, 7, 10, 14, 21, or 28 days. In particular embodiments, the one or more synthetic human milk oligosaccharides are administered at least once daily for at least 3, 5, 7, 10, 14, 21, or 28 days. In some embodiments, the one or more synthetic human milk oligosaccharides are administered in an amount of at least 2 g, 5 g, 10 g, 15 g, 20 g, 22 g, or 25 g of total human milk oligosaccharides per day. In certain embodiments, the one or more synthetic human milk oligosaccharides are administered are an amount of from 10 g to 25 g of total human milk oligosaccharides per day.

In particular embodiments, the B. longum subsp. infantis was previously administered to the subject in an amount of at least 5×10⁶ colony forming units (CFU) per day for at least 3 days. In some embodiments, the B. longum subsp. infantis was previously administered to the subject in an amount of at least 1×10⁸ colony forming units (CFU) per day for at least 7 days, 9 days, or 14 days. In certain embodiments, the concentrated human milk permeate composition comprises at least 10, at least 25, at least 50, or at least 100 human milk oligosaccharides, and the human milk oligosaccharides comprise 2′-fucosyllactose, 3-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose.

In particular embodiments, the concentrated human milk permeate composition was previously administered in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day for at least 3 days. In some embodiments, the concentrated human milk permeate composition was previously administered in an amount from 10 g to 25 g of total human milk oligosaccharides for at least 7, 9, or 14 days. In certain embodiments, the B. longum subsp. infantis and the concentrated human milk permeate composition were previously administered to the subject on the same day for at least 3 days, 5 days, 7 days, 9 days, or 14 days.

Particular embodiments provide a method for maintaining engraftment of a probiotic strain of B. longum subsp. infantis in a subject in need thereof to treat or prevent a disease, disorder, or condition associated with one or more of dysbiosis of the intestinal microbiome, inflammation, infection, allergy, or immune dysfunction in the subject, the method comprising administering to the subject one or more synthetic human milk oligosaccharides in an amount of from 10 g to 25 g of total human milk oligosaccharides per day for at least 7 days, wherein the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose; wherein the subject has previously been administered the B. longum subsp. infantis and a concentrated human milk permeate composition comprising human milk oligosaccharides; wherein the subject was previously administered at least 1×10⁸ colony forming units (CFU) per day of the B. longum subsp. infantis for at least 7 days; wherein the subject was previously administered the concentrated human milk permeate composition in an amount of at least 10 g of total human milk oligosaccharides per day for at least 7 days; and wherein the B. longum subsp. infantis and the concentrated human milk permeate composition were previously administered to the subject on the same day for at least 3 days, 5 days, or 7 days. Certain embodiments further comprise administering at least one dose of the concentrated human milk permeate after at least one dose of the one or more synthetic human milk oligosaccharides have been administered. In particular embodiments, the at least once of dose of the concentrated human milk permeate composition is administered at least once between doses of the one or more synthetic human milk oligosaccharides.

Also provided herein is a method for treating or preventing a disease, disorder, or condition in a subject in need thereof, the method comprising administering to the subject (i) a concentrated human milk permeate composition comprising human milk oligosaccharides; (ii) at least one probiotic strain of B. longum subsp. infantis; and (iii) one or more synthetic human milk oligosaccharides; wherein the one or more synthetic human milk oligosaccharides are administered at least once on a day that occurs after a day wherein B. longum subsp. infantis is administered; and wherein the one or more synthetic human milk oligosaccharides are administered at least once on a day that the concentrated human milk permeate composition is not administered.

In some embodiments the concentrated human milk permeate composition is obtained from human milk permeate that results from ultrafiltration of human skim milk, wherein the human skim milk is obtained by removing cream from pooled human milk, and wherein the pooled human milk is pooled from the milk of at least 50, 100, or 150 human milk donors; and wherein the concentrated human milk permeate composition comprises at least 10, at least 25, at least 50, or at least 100 human milk oligosaccharides.

In certain embodiments, the concentrated human milk permeate composition is administered in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day for at least 3 days. In particular embodiments, the concentrated human milk permeate composition is administered in an amount from 10 g to 25 g of total human milk oligosaccharides per day for at least 7, 9, or 14 days. In particular embodiments, the B. longum subsp. infantis is administered to the subject in an amount of at least 5×10⁶ colony forming units (CFU) per day for at least 3 days. In some embodiments, the B. longum subsp. infantis is administered to the subject in an amount of at least 1×10⁸ colony forming units (CFU) per day for at least 7 days, 9 days, or 14 days. In certain embodiments, the B. longum subsp. infantis and the concentrated human milk permeate composition are administered to the subject on the same day for at least 3 days, 5 days, 7 days, 9 days, or 14 days.

In particular embodiments, the one or more synthetic human milk oligosaccharides comprises one or more of 2′-fucosyllactose, 3-fucosyllactose, 3′-sialyllactose, 6′-sialyllactose, lacto-N-tetraose, lacto-N-neotetraose, or difucosyllactose; optionally one or two or more of 2′-fucosyllactose, 3-fucosyllactose, lacto-N-tetraose, or lacto-N-neotetraose. In some embodiments, the one or more synthetic human milk oligosaccharides are administered at least once every other day or at least once daily for at least 3, 5, 7, 10, 14, 21, or 28 days. In certain embodiments, the one or more synthetic human milk oligosaccharides are administered in an amount of at least 2 g, 5 g, 10 g, 15 g, 20 g, 22 g, or 25 g of total human milk oligosaccharides per day, optionally in an amount of from 10 g to 25 g of total human milk oligosaccharides per day.

In addition, provided herein method for treating or preventing a disease, disorder, or condition associated with one or more of dysbiosis of the intestinal microbiome, inflammation, infection, allergy, or immune dysfunction in a subject in need thereof comprising administering a probiotic strain of B. longum subsp. infantis, wherein the method comprises two or more treatment phases comprising at least a colonization phase and at least one subsequent maintenance phase; wherein the colonization phase comprises administering to the subject (i) the B. longum subsp. infantis and (ii) a concentrated human milk permeate composition comprising human milk oligosaccharides; and wherein the at least one maintenance phase comprises administering to the subject one or more synthetic human milk oligosaccharides, wherein the B. longum subsp. infantis can be detected within the subject's intestinal microbiome throughout the duration of the at least one maintenance phase.

Also provided is a method for treating or preventing graft versus host disease in a subject in need thereof comprising administering a probiotic strain of B. longum subsp. infantis, wherein the method comprises two or more treatment phases comprising at least a colonization phase and at least one subsequent maintenance phase; wherein the colonization phase comprises administering to the subject (i) B. longum subsp. infantis and (ii) a concentrated human milk permeate composition comprising human milk oligosaccharide; and wherein the at least one maintenance phase comprises administering to the subject one or more synthetic human milk oligosaccharides, wherein the B. longum subsp. infantis can be detected within the subject's intestinal microbiome throughout the duration of the at least one maintenance phase.

In particular embodiments, the subject receives an allogenic hematopoietic stem cell transplant, and wherein the colonization phase takes place beginning at least 7, 14, or 21 days prior to the allogenic hematopoietic stem cell transplant and lasts at least until 7, 14, 21, 28, 35 days after the allogenic hematopoietic stem cell transplant. In some embodiments, the subject receives treatment with antibiotics beginning at least 5 days prior to the allogenic hematopoietic stem cell transplant lasting until at least 5 days after the allogenic hematopoietic stem cell transplant, optionally wherein the antibiotics comprises one or more of a fourth-generation cephalosporins, a glycopeptide, a piperacillin-tazobactam, a carbapenem, an aminoglycoside, or a quinolone; and wherein the colonization phase lasts until at least 10 days after the end of the treatment with antibiotics. In certain embodiments, the concentrated human milk permeate composition is obtained from human milk permeate that results from ultrafiltration of human skim milk, wherein the human skim milk is obtained by removing cream from pooled human milk, and wherein the pooled human milk is pooled from the milk of multiple human milk donors, wherein the pooled human milk is pooled from the milk of at least 50, 100, or 150 human milk donors; and wherein the concentrated human milk permeate composition comprises at least 10, at least 25, at least 50, or at least 100 human milk oligosaccharides.

In particular embodiments, the colonization phase comprises a duration of at least 3 days, 5 days, 7 days, 9 days, or 14 days. In some embodiments, the B. longum subsp. infantis is administered to the subject at least once every other day or at least once daily during the colonization phase. In certain embodiments, the B. longum subsp. infantis is administered in an amount of at least 5×10⁶ colony forming units (CFU) per day during the colonization phase In particular embodiments, B. longum subsp. infantis is administered to the subject in an amount of at least 1×10⁸ colony forming units (CFU) per day during the colonization phase. In some embodiments, the concentrated human milk permeate composition is administered to the subject at least three times, five times, seven times, nine times, ten times or fourteen times during the colonization phase. In certain embodiments, the concentrated human milk permeate composition is administered at least once every two days or at least once daily during the colonization phase. In particular embodiments, the B. longum subsp. infantis and the concentrated human milk permeate composition are administered on the same day at least once, three times, five times, seven times, nine times, or fourteen times during the colonization phase.

In some embodiments, the concentrated human milk permeate composition is administered to the subject in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day. In certain embodiments, the B. longum subsp. infantis (i) is detectable within the subject's intestinal microbiome at the end of the colonization phase; and/or (ii) is detectable at a greater amount and/or as a greater portion of the total microbiota of the subject's intestinal microbiome at the end of the colonization phase than what is detectable prior to and/or on the first day of the colonization phase. In particular embodiments, the one or more synthetic human milk oligosaccharides are administered at least once during the colonization phase.

In some embodiments, the maintenance phase comprises a duration of at least 3 days, 5 days, 7 days, 9 days, 14 days, 21 days, or 28 days or 3 months. In certain embodiments, the maintenance phase comprises administering to the subject the one or more synthetic human milk oligosaccharides at least once every two days or at least once daily. In particular embodiments, the one or more synthetic human milk oligosaccharides are administered in an amount of at least 1 g, 2 g, 3 g, 4 g, 4.5 g, 5 g, 6 g, 7 g, 8 g, 9 g, 10 g, 12 g, 15 g, 18 g, 20 g, 22 g, or 25 g of total human milk oligosaccharides per day. In some embodiments, the one or more synthetic human milk oligosaccharides are administered in an amount from 10 g to 25 g of total human milk oligosaccharides. In certain embodiments, the one or more synthetic human milk oligosaccharides comprises one or more of 2′-fucosyllactose, 3-fucosyllactose, 3′-sialyllactose, 6′-sialyllactose, lacto-N-tetraose, lacto-N-neotetraose, or difucosyllactose; optionally one or two or more of 2′-fucosyllactose, 3-fucosyllactose, lacto-N-tetraose, or lacto-N-neotetraose. In particular embodiments, the concentrated human milk permeate composition is administered at least once, three times, five times, seven times, nine times, ten times, or fourteen times during the maintenance phase. In some embodiments, the B. longum subsp. infantis is administered at least once, three times, five times, seven times, nine times, ten times, or fourteen times during the maintenance phase.

In certain embodiments, the colonization phase and the maintenance phase are repeated in two or more cycles, optionally wherein the cycles are repeated after a rest period comprising at least one, three, seven, or fourteen days.

### BRIEF DESCRIPTION OF THE DRAWINGS

FIG. 1 provides a graph displaying optical density measurements at 600 nm (OD600) collected at regular intervals spaced 30 minutes apart from cultures of three exemplary strains of B. longum subsp. infantis incubated in the presence of 2′-fucosyllactose and lacto-N-neotetraose.

### DETAILED DESCRIPTION

Provided herein are compositions, kits, and articles of manufacture, as well as methods of use thereof, that contain one or more prebiotics, e.g., synthetic human milk oligosaccharides and concentrated human milk permeate compositions that contain human milk oligosaccharides, and at least one probiotic strain of bacterium, e.g., a Bifidobacterium such as B. longum subsp. infantis, capable of consuming human milk oligosaccharides. In certain aspects, the provided compositions, kits, and articles of manufacture are particularly useful in the treatment or prevention of diseases or conditions associated with inflammation, allergies, or immune disorders. In some aspects, the provided compositions, kits, and articles of manufacture are useful to treat or prevent dysbiosis, e.g., of the intestinal microbiome, as well as diseases or disorders that may originate from, cause, or are otherwise associated with dysbiosis. In some embodiments, the provided compositions, kits, and articles of manufacture are useful for the treatment or prevention of graft versus host disease (GVHD).

In certain aspects, the maintenance of a healthy human metabolism depends on a symbiotic consortium among bacteria, archaea, viruses, fungi, and host eukaryotic cells throughout the human gastrointestinal tract. For example, microbial communities may provide enzymatic machinery and metabolic pathways that contribute to food digestion, xenobiotic metabolism, and production of a variety of bioactive molecules. Disturbances to the microbiome may result in a microbial imbalance (dysbiosis) characterized by phylum-level changes in the microbiota composition, including a marked decrease in the representation of obligate anaerobic bacteria and an increased relative abundance of facultative anaerobic bacteria. While dysbiosis is associated with numerous diseases and conditions, successfully treating dysbiosis is difficult, particularly in vulnerable or immunocompromised patients.

The provided compositions, methods, kits, and articles of manufacture address these needs. In particular, the present invention includes specific combinations of prebiotics, e.g., synthetic human milk oligosaccharides and/or concentrated human milk permeate compositions containing human milk oligosaccharides, and one or more probiotic strains of bacteria, such as strains that internalize and consume human milk oligosaccharides, e.g., Bifidobacterium longum subspecies (subsp.) infantis (also referred to herein as B. longum subsp. infantis or B. infantis). The synbiotic combinations of prebiotics and probiotics are particularly safe and effective for treating, ameliorating, or reducing dysbiosis in the gut microbiome as well as effective in treating, ameliorating, or preventing diseases or disorders that may be accompanied by dysbiosis, such including but not limited to diseases associated with immune disorders, inflammatory disorders, or infection.

In particular embodiments, at least one probiotic strain of bacterium (e.g., B. longum subsp. infantis) is administered in combination with a prebiotic, e.g., a concentrated human milk permeate composition containing human milk oligosaccharides, to a subject in need thereof. In some embodiments, the prebiotics selectively promote the engraftment and expansion of the probiotic strain within the subject's gut and/or intestinal microbiome. In certain embodiments, prebiotics, e.g., synthetic human milk oligosaccharides, are administered, e.g., daily, after the probiotic strain has been administered to maintain the presence, engraftment, growth, and/or viability of the probiotic within the subject's gut and/or intestinal microbiome.

In certain aspects, administration of the provided prebiotics and probiotics promotes an environment capable of promoting or allowing the growth or expansion of other beneficial microbiota within the subject's microbiome and/or preventing the growth or expansion of potentially pathogenic bacteria. For example, in some aspects, the provided prebiotics and probiotics, e.g., the probiotic strain and a concentrated human milk permeate composition and/or synthetic human milk oligosaccharides, are administered for a limited period of time, during which time the probiotic may generate or promote an environment, such as by influencing pH and/or producing short chain fatty acids, which impairs growth of pathogenic microbiota while promoting beneficial microbiota. Prebiotics, e.g., synthetic human milk oligosaccharides, may then be administered or continue to be administered to maintain the presence, colonization, or engraftment of the probiotic strain within the subject's gut and/or intestinal microbiome. In certain aspects, after such a period of time, administration of the prebiotics may be withdrawn, thereby reducing the presence of the provided probiotic in the subject's microbiome. In some such aspects, the expanded presence of the beneficial microbiota may continue to sustain this healthy environment even when the provided probiotic is no longer detectable, thereby maintaining a healthy microbiome and/or preventing dysbiosis after the treatment is completed.

In various aspects, the provided combination of prebiotics and probiotics have several advantages over alternative treatments that target the microbiome. For example, in some aspects, the provided prebiotics, e.g., the synthetic human milk oligosaccharides and the concentrated human milk permeate composition, provide a selective carbon and/or energy source for the beneficial probiotic strain, e.g., B. longum subsp. infantis, that is not typically present in the healthy adult microbiome. Thus, in contrast to treatments such as fecal microbiota transplants (FMTs), the engraftment, expansion, and presence of the probiotic strain in the subject's microbiome may be controlled by the concurrent or subsequent administration of the provided prebiotics. For example, in some aspects, the expansion of the probiotic strain may be increased by increasing or extending the administration of the provided prebiotics. In some aspects, the duration of time that the probiotic strain is present within the subject's microbiome may be controlled by withdrawing or terminating administration of the prebiotic, without any need for antibiotics.

In some aspects, the probiotic strain is or includes B. longum subsp. infantis. In infants, breast feeding may result in the expansion of B. longum subsp. infantis and a subsequent reduction in other potentially deleterious species, e.g. species or strains of Enterobacteriaceae. However, B. longum subsp. infantis is not typically present in the healthy adult microbiome, nor are human milk oligosaccharides typically present in an adult diet. In certain aspects, prior to the instant invention it was not clear what, if any, benefits of B. longum subsp. infantis engraftment could have for adult health. The present invention relates, at least in part, to the surprising finding that B. longum subsp. infantis can indeed be engrafted in the adult intestinal microbiome when administered along with human milk oligosaccharides. Engraftment of B. longum subsp. infantis through this manner results in surprisingly beneficial effects for adult diseases and conditions, at least in part through one or more of a reduction of deleterious bacterial species, an increased production of short chain fatty acids, and reduction of inflammation or pro-inflammatory factors.

In some embodiments, provided herein are combinations of prebiotics, e.g., synthetic human milk oligosaccharides and concentrated human milk permeate compositions containing human milk oligosaccharides, and probiotics, e.g., strains of Bifidobacterium such as B. longum subsp. infantis. In some embodiments, this synbiotic prebiotic and probiotic combination synergistically (i) promotes engraftment and expansion of the probiotic; (ii) improves, reduces, treats, or ameliorates dysbiosis; (iii) promotes diversity (e.g., alpha and/or beta diversity) of the gut microbiome; (iv) promotes production of short chain fatty acids; and/or (v) reduces, improves, treats, or ameliorates inflammation or conditions associated with auto- or hyper-immunity. In certain aspects, such effects may be achieved, inter alia, by the production of lactate or acetate; reduction of intestinal pH; and/or cross-feeding of butyrate producers by the probiotic strain as selectively promoted by the prebiotic.

In some embodiments, the probiotic strain, e.g., B. longum subsp. infantis, is capable of internalizing human milk oligosaccharides, such as for internal metabolism and/or hydrolysis of all or some of the prebiotics. In some aspects, probiotic strains capable of internalizing human milk oligosaccharides may have endogenous transport or import molecules as well as glycosyl hydrolases to deconstruct oligosaccharides having certain specific glycosidic bonds or linkages found in human milk oligosaccharides. In some aspects, the ability to internalize and metabolize human milk oligosaccharides may allow for these probiotic strains to be uniquely successful in colonizing the gut of a subject administered human milk oligosaccharides, e.g., because human milk oligosaccharides are uniquely consumed by these bacteria as opposed to other bacteria present within the subject's microbiome. Because the human milk oligosaccharides are internalized within the cells, breakdown products, e.g., monosaccharides, do not diffuse and/or are not consumed by other bacteria. Thus, in some embodiments, the prebiotics, e.g., of human milk oligosaccharides, selectively promote growth and expansion of the probiotic strain over other bacteria, e.g., present in the gut and/or microbiome.

In some aspects, an advantage of the provided compositions is that the human milk oligosaccharides selectively promote the growth and expansion of the probiotic strain, e.g., B. longum subsp. infantis, in the human gut or intestinal microbiome in vivo. Particular embodiments contemplate that, in some cases, the probiotic strain may consume or internalize certain oligosaccharides in some environments, such as in vitro assays or in the gut or microbiome of non-human animals in vivo, but not in a human gut or microbiome. Thus, in some embodiments, while many kinds of oligosaccharides may promote the growth of the probiotic strain in vitro, the provided human milk oligosaccharides promotes the engraftment, growth, and expansion of the provided probiotic stain in the human gut or intestinal microbiome in vivo.

In certain aspects, the provided compositions and methods successfully treat a subject with dysbiosis or a disorder or disease related to or associated with dysbiosis, through the combination of one or more probiotic strains and prebiotics that are selectively consumed by the probiotic strain, thereby promoting or facilitating engraftment in the subject's, e.g., the adult subject's, intestinal microbiome. Surprisingly, administration of the prebiotic compositions provided herein, e.g., concentrated human milk permeate compositions and/or synthetic human milk oligosaccharides, result in engraftment, colonization, and/or an expansion of the probiotic strain that may be detected days or weeks after the probiotic strain has been administered, and further, that may be maintained for prolonged periods so long as the prebiotics continue to be administered. Thus, in certain embodiments, the provided prebiotic compositions are surprisingly effective at supporting the engraftment, growth, colonization, expansion, and/or the persistence of the probiotic strain in the subject's gut or intestinal microbiome.

In certain embodiments, provided herein is an improved strategy for treating diseases or conditions, e.g., those relating to inflammation, immune dysfunction, dysbiosis of the intestinal microbiome, by pairing the administration of a probiotic with a prebiotic, e.g., carbon source, that is selectively utilized by the probiotic with respect to microflora typically present in a healthy or dysbiotic human intestinal microbiome. Particular aspects contemplate that this strategy may be achieved with any combination of probiotic bacteria and prebiotics that are selectively consumed by the probiotic, providing that the probiotic has one or more features discussed herein, e.g., SCFA production, pH regulation, etc., thought to treat, reduce, or ameliorate dysbiosis of the intestinal microbiome or conditions or diseases, e.g., relating to dysbiosis, inflammation, or immune dysfunction, with a prebiotic that is selectively consumed by the probiotic. Certain embodiments contemplate that additional prebiotic/probiotic combinations not explicitly disclosed herein may be identified by routine methods and techniques along with the guidance provided herein.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. METHODS OF TREATING DISEASES ASSOCIATED WITH DYSBIOSIS

In particular embodiments, provided herein are methods for treating, preventing, or ameliorating one or more diseases, disorders, or conditions in a subject in need thereof, e.g., diseases that arise from and/or are associated with dysbiosis of the intestinal microbiome. In certain embodiments, the methods are or include synbiotic treatment regimens, where at least one probiotic strain of bacteria, e.g., B. longum subsp. infantis, and one or more prebiotics that are selectively consumed by the at least one probiotic strain are administered. In particular embodiments, the at least one probiotic strain is any described herein, e.g., in Section II-C or listed in Table I. In some embodiments, the prebiotics are or include human milk oligosaccharides. In certain embodiments, the human milk oligosaccharides are or include those purified or isolated from human milk, e.g., such as a concentrated human milk permeate composition that contains human milk oligosaccharides, such as any described herein, e.g., in Section II-A. In particular embodiments, the human milk oligosaccharides are synthetic, such as any of the synthetic human milk oligosaccharides described herein e.g., in Section II-B.

In some embodiments, the methods are or include steps for administering to the subject a concentrated human milk permeate composition, such as any described herein, e.g., in Section II-A; one or more synthetic oligosaccharides, such as one or more synthetic human milk oligosaccharides described herein e.g., in Section II-B; and at least one probiotic strain of bacterium, such as a probiotic strain described herein, e.g., in Section II-C or listed in Table 1. In some embodiments, the at least one probiotic strain and one or both of the concentrated human milk permeate composition and the synthetic human milk oligosaccharides are administered. In certain embodiments, the concentrated human milk permeate composition and the synthetic human milk oligosaccharides are administered together. In particular embodiments, the concentrated human milk permeate composition and the synthetic human milk oligosaccharides are administered separately, e.g., in separate doses and/or on separate days, or during different dosing or treatment phases, such as during a treatment regimen.

In particular embodiments, the concentrated human milk permeate composition containing human milk oligosaccharides and/or the one or more synthetic human milk oligosaccharides are administered along with at least one probiotic strain, e.g., a probiotic bacterium capable of consuming human milk oligosaccharides, to a subject in need thereof to treat or prevent a disease, condition, or disorder. In various embodiments, the disease, disorder, or condition is any one or more of those described herein, e.g., in Section-III. In certain embodiments, one or more synthetic human milk oligosaccharides and a probiotic bacterium capable of consuming human milk oligosaccharides are administered to a subject to treat or prevent the disease, disorder, or condition.

In certain embodiments, provided herein are methods for treating, preventing, or ameliorating one or more diseases, disorders, or conditions that are or may be associated with dysbiosis, e.g., of the intestinal microbiome, in a subject in need thereof. In certain embodiments, the methods provide administering the provided prebiotic and probiotic compositions to a subject in need thereof.

In some aspects, the intestinal microbiome is involved in or associated with a number of physiological functions including digestion, metabolism, extraction of nutrients, synthesis of vitamins, prevention of pathogen colonization, and immune modulation. In some such aspects, alterations or changes in composition and biodiversity of the intestinal microbiome may be associated with or exacerbate various metabolic states, gastrointestinal disorders, and other pathophysiological conditions. In some aspects, conditions, diseases, or disorders with inflammatory components or components relating to infection, allergy, or immune dysfunction may be exacerbated by dysbiosis or may have an underlying contribution of dysbiosis to the pathology. Thus, in certain aspects, targeting the microbiome with the provided prebiotic and probiotic compositions may successfully treat, alleviate, or prevent a wide range of conditions, diseases, and disorders.

In some embodiments, provided herein is a method for treating, reducing, ameliorating, or preventing dysbiosis. In particular embodiments, the method is or includes steps for administering to the subject a concentrated human milk permeate composition, such as any described herein e.g., in Section II-A; one or more synthetic oligosaccharides, e.g., such as any of the oligosaccharides described in Section II-B; and at least one probiotic strain, such as a probiotic strain described herein, e.g., in Section II-C or listed in Table 1.

In some embodiments, the one or more diseases or conditions is, includes, or is associated with dysbiosis, e.g., of the intestinal microbiome. In certain embodiments, the microbiome is an intestinal microbiome of a human. In certain embodiments, the microbiome is a gut or intestinal microbiome of an adult human. In certain embodiments, the one or more diseases or conditions is, includes, or is associated with inflammation. In particular embodiments, the one or more diseases or conditions is, includes, or is associated with an autoimmune disease. In particular embodiments, the one or more diseases is or is associated with an allergy. In certain embodiments, the prebiotics, e.g., the concentrated human milk permeate composition and/or the one or more synthetic human milk oligosaccharides, and the at least one probiotic strain, e.g., B. longum subsp. infantis, are administered to prevent a disease, disorder, or condition. In some embodiments, the prebiotics and the at least one probiotic strain prevent a condition described herein, e.g., in Section III. In particular embodiments, the prebiotics and the at least one probiotic strain reduce the risk, likelihood, or probability of the disease, disorder, or condition, and/or of experiencing one or more symptoms associated with the disease, disorder, or condition. In some embodiments, the risk, likelihood, or probability is reduced by at least 10%, 20%, 25%, 30%, 40%, 50%, 60%, 70%, 75%, 80%, 90%, 99%, or 99.9% as compared to alternative treatments or no treatments, or as compared to administration of the at least one probiotic strain or prebiotics alone.

As used herein, “subject” and “subject in need thereof” are used interchangeably. In particular embodiments, the subject is a human. In some embodiments, the subject is an infant, a child, a juvenile, or an adult. In certain embodiments, the subject is at least 1 month, 3 months, 6 months, 12 months, 18 months, or 24 months of age. In certain embodiments, the subject is at least 1 year, 2 years, 5 years, 10 years, 12 years, 16 years, or at least 18 years of age. In some embodiments, the subject is at least 12 years old. In certain embodiments, the subject is at least 18 years old. In some embodiments, the subject is an adult. In certain embodiments, the subject is elderly, e.g., at least 65, 70, or 75 years of age. In certain embodiments, the subject has, is suspected of having, or is at risk for a condition, disease, or disorder described herein, e.g., in Section III.

In some embodiments, the administration of the prebiotics, e.g., the concentrated human milk permeate composition and/or the synthetic oligosaccharides, allows for the engraftment and expansion of the at least one probiotic strain, e.g., B. longum subsp. infantis. In certain embodiments, the at least one probiotic strain is exogenous to the subject's gut or intestinal microbiome. In particular embodiments, the at least one probiotic strain is not present within the subject's gut or intestinal microbiome prior to its administration. In certain embodiments, the prebiotics, e.g., the concentrated human milk permeate composition and/or the one or more synthetic oligosaccharides, are administered concurrently with and/or subsequently to administration of the at least one probiotic strain. In some embodiments, the at least one probiotic strain is present and/or expands within the subject's microbiome during a time period in which the prebiotics are administered. In certain embodiments, the presence or amount of the at least one probiotic strain within the subject's gut or intestinal microbiome is reduced when administration of the prebiotics ends, is ceased, or is terminated. In particular embodiments, the probiotic strain is absent and/or undetectable following the termination or end of administration of the prebiotics, e.g., within days or weeks of the termination or end. In certain embodiments, the presence of the probiotic strain, e.g., B. longum subsp. infants, is transient and is regulated by administration of the prebiotics.

In some embodiments, the prebiotics, e.g., the concentrated human milk permeate composition and/or the synthetic oligosaccharides, provide an energy and/or a carbon source selectively or exclusively to the probiotic strain, e.g., B. longum subsp. infantis, such that it promotes growth or expansion of the probiotic strain, e.g., in vivo in the gut or within the microbiome. In certain embodiments, the prebiotics and the at least one probiotic strain are administered in a manner sufficient for the probiotic strain to engraft, grow, expand, or establish itself within the microbiome of the subject. In particular embodiments, the administration of the prebiotics and the at least one probiotic strain results in an increase in the levels and/or production of lactate, acetate, and/or short chain fatty acid (SCFA) in the gut that is synergistic, e.g., greater than what would be expected based on administration of the prebiotics or probiotic strain alone.

In certain embodiments, the expansion, level, or amount of the at least one probiotic strain, e.g., B. longum subsp. infantis, may be regulated by the administration of the prebiotics, e.g., the concentrated human milk permeate composition and/or the one or more synthetic oligosaccharides. Thus, in some aspects, the at least one probiotic strain is administered to the subject, and the concurrent or subsequent administration of the concentrated human milk permeate composition and/or the synthetic human milk oligosaccharides may be adjusted to provide a therapeutic response, e.g., to promote growth or expansion of beneficial microbiota and/or reduce symptoms associated with a disease, disorder, or condition relating to dysbiosis. In some embodiments, the dosage and/or duration of treatment with the concentrated human milk permeate composition and/or the synthetic human milk oligosaccharides can depend on several factors, including severity and responsiveness of the disease, route of administration, time course of treatment (days to months to years), and time to amelioration of the disease. In certain embodiments, the at least one probiotic strain is administered to the subject along with the concentrated human milk permeate composition, which supports or promotes engraftment of the probiotic strain, then, after one or more doses, the one or more synthetic oligosaccharides are administered to maintain engraftment of the probiotic.

In particular embodiments, the provided methods are or include a treatment regimen. In some embodiments, the concentrated human milk permeate composition and the at least one probiotic strain, e.g., B. longum subsp. infantis, are administered together to the subject. In certain embodiments, the concentrated human milk permeate composition and the at least one probiotic strain are administered together once to the subject. In particular embodiments, the concentrated human milk permeate composition and the at least one probiotic are administered together multiple times to the subject. In some embodiments, the concentrated human milk permeate composition and the at least one probiotic strain are administered once, twice, three times, four times, five times or more than five times per month; once, twice, three times, four times, five times, six times, seven times, or more than seven times per week; or once, twice, or more than twice daily. In some embodiments, the concentrated human milk permeate composition and the at least one probiotic strain are administered multiple times during a regimen lasting for, for about, or for at least, one week, two weeks, three weeks, four weeks, five weeks, ten weeks, one month, two months, three months, six months, or twelve months.

In particular embodiments, the concentrated human milk permeate composition and the at least one probiotic strain, e.g., B. longum subsp. infantis, are administered to a subject in need thereof after the subject has underwent an allogenic transplant, e.g., an allogenic bone marrow transplant (BMT) or hematopoietic stem cell transplant (HSCT). In some embodiments, the subject undergoes a treatment with antibiotics, and the prebiotics, e.g., the concentrated human milk permeate composition and/or the one or more synthetic oligosaccharides, and the at least one probiotic strain, e.g., B. longum subsp. infantis, are administered to a subject immediately after the treatment with antibiotics is completed. In certain embodiments, the subject undergoes a treatment with antibiotics, and treatment with the prebiotics and the at least one probiotic strain is initiated during the antibiotic treatment.

In particular embodiments, the concentrated human milk permeate composition and the at least one probiotic strain, e.g., B. longum subsp. infantis, are administered separately to the subject. In certain embodiments, the at least one probiotic strain and the concentrated human milk permeate composition are administered both together and separately during the same treatment regimen. For example, in some embodiments the at least one probiotic strain and the prebiotics are administered together initially over one or more days, e.g., treatment days, and then one or both of the at least one probiotic strain and the prebiotics are administered alone over one or more subsequent days, e.g., treatment days. In some instances, the at least one probiotic strain is administered with the concentrated human milk permeate composition initially during the treatment regimen (such as during an initial or first treatment phase), and later in the regimen the concentrated permeate composition is administered in the absence of the at least one probiotic strain (such as during a second or subsequent treatment phase).

In certain embodiments, one or more synthetic oligosaccharides are administered after the at least one probiotic strain, e.g., B. longum subsp. infantis, has been administered, e.g., to prolong or maintain engraftment of the probiotic strain. In some embodiments, the one or more synthetic oligosaccharides are administered after the concentrated human milk permeate composition has been administered, e.g., to prolong or maintain engraftment of the probiotic strain. For example, in some embodiments, the at least one probiotic strain and the concentrated human milk permeate composition are administered together initially over one or more treatment days (such as during a first treatment phase), and then one or more synthetic oligosaccharides are administered over one or more subsequent treatment days (such as during a subsequent treatment phase).

### A.) Maintaining Colonization or Engraftment

In certain embodiments, diseases, disorders, or conditions are treated or prevented by prolonging or maintaining the colonization or engraftment of an administered probiotic. Particular aspects contemplate that prolonging the colonization or engraftment of the at least one probiotic strain may promote the growth or expansion of beneficial gut microbiota, impair growth or expansion of pathogenic bacteria, and/or reduce inflammation. In certain embodiments, the engraftment or colonization of an administered probiotic, e.g., B. longum subsp. infantis, treats, ameliorates, prevents, or reduces the likelihood or severity of any disease, disorder, or condition, and/or any one or more symptoms thereof, of any of those described herein, e.g., in Section-III.

In some embodiments, the at least one probiotic strain and the concentrated human milk permeate composition are administered to a subject to promote engraftment of the probiotic strain, e.g., within the subject's gut or intestinal microbiome. In some embodiments, the administration of the concentrated human milk permeate composition may be continued for a period of time after the at least one probiotic strain has been administered, to promote or establish engraftment of the probiotic strain. In some embodiments, once engraftment of the probiotic strain is established, one or more synthetic human milk oligosaccharides are administered, optionally in the absence of the concentrated human milk permeate composition, to maintain the engraftment of the probiotic strain, e.g., within the subject's gut or intestinal microbiome. In certain embodiments, the one or more synthetic human milk oligosaccharides are administered over a period or treatment phase of at least 1 day, 3 days, 7 days, 14 days, 28 days, 1 month, 3 months, 6 months, or longer to maintain the engraftment.

Particular embodiments contemplate that engraftment may be detected or determined by routine methods; non-limiting examples include detection of nucleic acids having a sequence of a portion of the probiotic strain's genomic DNA in stool of the subject (e.g., by quantitative PCR). In some aspects, B. longum subsp. infantis is considered to be exogenous to the adult human microbiome. In some aspects, colonization and/or engraftment may be determined by by detection of the probiotic strain (e.g., by quantitative PCR) in stool samples collected from the subject at levels higher than what would be expected based on the dose administered to the subject, and/or the detection or presence of the probiotic strain in stool collected on a day when the probiotic strain was not administered. Routine methods may include, but are not limited to, any of those described herein, e.g., in the Examples.

In some embodiments, the one or more synthetic oligosaccharides, e.g., synthetic human milk oligosaccharides, are administered to a subject who has previously been administered the at least one probiotic strain, e.g., B. longum subsp. infantis. In certain embodiments, the subject was previously administered the at least one probiotic strain and the concentrated human milk permeate composition. In some embodiments, the at least one probiotic strain and/or the concentrated human milk permeate composition were administered to the subject over one or more days. In some embodiments, the at least one probiotic strain was previously administered to subject, e.g., at least once every other day or daily, for at least 1, 3, 5, 7, 10, 14, or 21 days, or at least 2, 3, 4, 6, or 8 weeks. In some embodiments, the at least one probiotic strain was previously administered in amounts of at least 1×10³, 1×10⁴, 1×10⁵, 1×10⁶, 5×10⁶, 1×10⁷, 1×10⁷, 5×10⁷, 1×10⁸, or 5×10⁸, 1×10⁹, 5×10⁹, 8×10⁹, 1×10¹⁰, 5×10¹⁰, or 1×10¹¹ colony forming units (CFU) per dose or per day. In particular embodiments, the at least one probiotic strain was administered in an amount of at least 5×10⁶ CFU per dose or per day. In certain embodiments, the concentrated human milk permeate composition was previously administered to the subject e.g., at least once every other day or daily, for at least 1, 3, 5, 7, 9, or 14 days or at least 1, 2, 3, 4, 6, or 8 weeks. In particular embodiments, the concentrated human milk permeate composition was administered in an amount of about or of at least 2 g, 4 g, 4.5 g, 5 g, 9 g, 10 g, 15 g, 18 g, 20 g, 22 g, 25 g, or 50 g per day, e.g., by total weight of the human milk oligosaccharides of the composition.

In certain embodiments, the one or more synthetic human milk oligosaccharides are administered to a subject who has previously been administered B. longum subsp. infantis in an amount of at least 1×10⁸ CFU per dose or per day for at least 3, 5, 7, 10, or 14 days. In particular embodiments, the one or more synthetic human milk oligosaccharides are administered to a subject who has previously been administered B. longum subsp. infantis and a concentrated human milk permeate composition in an amount from 10 g to 25 g total human milk oligosaccharides per day for at least 3, 5, 7, 10, or 14 days.

In certain embodiments, the one or more synthetic oligosaccharides, e.g., human milk oligosaccharides, and the concentrated human milk permeate composition are administered to a subject who has previously been administered the at least one probiotic strain, e.g., B. longum subsp. infantis, e.g., to maintain or prolong colonization or engraftment. In certain embodiments, the one or more synthetic oligosaccharides and the concentrated human milk permeate composition are administered in alternating doses or on alternating days. In particular embodiments, the synthetic oligosaccharides are administered every day except for every two, three, four, five, seven, or fourteen days or more, when the concentrated human milk permeate composition is administered. In some embodiments, the one or more synthetic oligosaccharides, e.g., human milk oligosaccharides, are administered every day except for every two, three, four, five, seven, or fourteen days or more, in an amount of at least 2 g, 4 g, 4.5 g, 5 g, 9 g, 10 g, 15 g, 18 g, 20 g, 22 g, 25 g, or 50 g per day, e.g., by total weight of the human milk oligosaccharides. In particular embodiments, the one or more synthetic oligosaccharides, e.g., human milk oligosaccharides, are administered in an amount of 10 g to 25 g total human milk oligosaccharides per day. In some embodiments, on alternate days or every two, three, four, five, seven, or fourteen or more days, the concentrated human milk permeate composition is administered in an amount of at least 2 g, 4 g, 4.5 g, 5 g, 9 g, 10 g, 15 g, 18 g, 20 g, 22 g, 25 g, or 50 g per day, e.g., by total weight of the human milk oligosaccharides. In particular embodiments, the concentrated human milk permeate composition is administered in an amount of 10 g to 25 g total human milk oligosaccharides per day.

In some embodiments, the concentrated human milk permeate composition is continued to be administered to the subject after administration of the probiotic strain has ended or been stopped, discontinued, ceased, or terminated, e.g., continued for at least 1 day, 3 days, 7 days, or 14 days. In particular embodiments, the at least one probiotic strain is continued to be administered to the subject after administration of the concentrated human milk permeate composition had ended or been stopped, discontinued, ceased, or terminated, e.g., continued for at least 1 day, 3 days, 7 days, or 14 days.

In certain embodiments, administration of the synthetic oligosaccharides, e.g., synthetic human milk oligosaccharides, is initiated after the administration of the at least one probiotic strain has ended or has been stopped, discontinued, ceased, or terminated. In particular embodiments, administration of the synthetic oligosaccharides is initiated after the administration of the concentrated human milk permeate composition has ended or has been stopped, discontinued, ceased, or terminated. In particular embodiments, administration of the synthetic oligosaccharides, e.g., synthetic human milk oligosaccharides, is initiated after the administration of the at least one probiotic strain and the concentrated human milk permeate composition has ended or has been stopped, discontinued, ceased, or terminated. In various embodiments, administration of the synthetic oligosaccharides is initiated after the concentrated human milk permeate composition has ended or has been stopped, discontinued, ceased, or terminated, but prior to when administration of the at least one probiotic strain has ended or has been stopped, discontinued, ceased, or terminated.

In some embodiments, synthetic oligosaccharides, e.g., synthetic human milk oligosaccharides such as those described herein e.g., in Section II-B, are administered to a subject to prolong or maintain the engraftment or colonization of a previously administered probiotic, e.g., B. longum subsp. infantis. In some embodiments, the subject has or is suspected of having a disease, condition, or disorder associated with dysbiosis of the intestinal microbiome, inflammation, infection, allergy, or immune dysfunction. In particular embodiments, the probiotic was administered to treat, prevent, and/or reduce the severity, risk, and/or likelihood of the disease, condition, or disorder and/or one or more symptoms of the disease, disorder, or condition. In certain embodiments, the disease, disorder, or condition is one or more of any of those described herein, e.g., in Section III. In some aspects, the synthetic oligosaccharides, e.g., synthetic human milk oligosaccharides, are administered to prolong or maintain the efficacy and/or the duration of the treatment. In certain aspects, the synthetic oligosaccharides, e.g., synthetic human milk oligosaccharides, are administered to prolong or maintain the efficacy of the probiotic, such as to promote the growth of beneficial bacterial and/or suppress the growth the pathogenic bacteria.

In some embodiments, the one or more synthetic oligosaccharides are synthetic human milk oligosaccharides, such as those described herein e.g., in Section II-B. In some embodiments, the one or more synthetic human milk oligosaccharides are administered to the subject to prolong or maintain the engraftment or colonization of a previously administered probiotic, e.g., B. longum subsp. infantis. In certain embodiments, the subject was previously administered the probiotic, e.g., as described herein such as in Section I-B. In particular embodiments, the subject was previously administered the probiotic and human milk oligosaccharides. In certain embodiments, the subject was previously administered the probiotic in conjunction with concentrated human milk permeate composition, e.g., as described herein such as in Section I-B.

In particular embodiments, the synthetic oligosaccharides, e.g., synthetic human milk oligosaccharides, are administered for at least 1, 2, 3, 4, 5, 6, 7, 10, 14, 21, or 28 days, or for at least 3 weeks, 4 weeks, 5 weeks, 6 weeks, 7 weeks, 8 weeks, 9 weeks, 10 weeks, or at least 2 months, 3 months, 4 months, 5 months, 6 months or more, e.g., to prolong or maintain engraftment or colonization of the probiotic (e.g., B. longum subsp. infantis). In some embodiments, the synthetic oligosaccharides are administered at least once a week, at least twice a week, at least three times a week, at least every other day, or at least daily, e.g., to prolong or maintain engraftment or colonization of the probiotic.

In some embodiments, the prolonging or maintenance of engraftment or colonization of the at least one probiotic strain with the subject's gut or intestinal microbiome may be confirmed through the measurement or detection of the probiotic strain at higher levels and/or greater amounts during a period of time in which the at least one probiotic strain is no longer administered to the subject than what would be measured in a different subject that was never administered the at least one probiotic strain and/or than what was or would have been measured in the subject prior to any administration of the at least one probiotic strain. Particular embodiments contemplate that for probiotic strains such as B. longum subsp. infantis that are exogenous to the subject's gut or intestinal microbiome, the detectable and/or identifiable presence of the probiotic during a time period, e.g., days or weeks, in which the at least one probiotic strain is no longer administered to the subject is sufficient to demonstrate the maintenance or prolonging of the probiotic strain's engraftment or colonization. In some such aspects, the presence of the probiotic strain within the subject's gut or intestinal microbiome, as well as the amount or level in which it is present, may be measured, detected, or identified directly or indirectly by routine methods, including any of those described herein, e.g., PCR based techniques described in the Examples.

In some embodiments, at least 1 g, 2 g, 3 g, 4 g, 4.5 g, 5 g, 6 g, 7 g, 8 g, 9 g, 10 g, 12 g, 15 g, 18 g, 20 g, 22 g, or 25 g per day of the one or more synthetic oligosaccharides, e.g., human milk oligosaccharides, are administered, e.g., to prolong or maintain engraftment or colonization of the at least one probiotic strain (e.g., B. longum subsp. infantis). In particular embodiments, from 0.5 g to 50 g, 1 g to 25 g, 2.5 g to 10 g, 5 g to 10 g, 10 g to 30 g, 10 g to 15 g, 15 g to 20 g, 20 g to 25 g, or 17.5 g to 22.5 g per day of the one or more synthetic oligosaccharides are administered, e.g., to maintain engraftment or colonization of the probiotic. In certain embodiments, at least or about 2 g, 4.5 g, 9 g, 18 g, or 22 g per day of one or more synthetic oligosaccharides are administered, e.g., to prolong or maintain engraftment or colonization of the probiotic. In some embodiments, from 10 g to 25 g per day of the synthetic human milk oligosaccharides are administered to prolong or maintain engraftment or colonization of the probiotic.

In particular embodiments, the synthetic oligosaccharides, e.g., human milk oligosaccharides, include any of those described herein, e.g., in Section II-B. In certain embodiments, the synthetic oligosaccharides are or include one or more of 2′-fucosyllactose, 3-fucosyllactose, Lacto-N-tetraose, or Lacto-N-neotetraose. In some embodiments, one or more of 2′-fucosyllactose, 3-fucosyllactose, Lacto-N-tetraose, or Lacto-N-neotetraose are administered at least once daily for at least 7, 10, 14, 21, 28, or 35 days, e.g., to maintain or prolong engraftment or colonization of the probiotic. In certain embodiments, at least or about 5 g, 10 g, 15 g, 18 g, 20 g, or 22 g of a mixture of one or more of 2′-fucosyllactose, 3-fucosyllactose, lacto-N-tetraose, or lacto-N-neotetraose are administered at least once daily for at least 7, 10, or 14 days. In particular embodiments, the one or more human milk oligosaccharides are 2′-fucosyllactose and lacto-N-neotetraose.

In some embodiments, the synthetic oligosaccharides, e.g., synthetic human milk oligosaccharides, are or include one or more of 2′-fucosyllactose, 3′-sialyllactose, 6′-sialyllactose, lacto-N-tetraose, lacto-N-neotetraose, and difucosyllactose. In certain embodiments, one or more of 2′-fucosyllactose, 3′-sialyllactose, 6′-sialyllactose, lacto-N-tetraose, lacto-N-neotetraose, and difucosyllactose are administered at least once daily for at least 7, 10, or 14 days, e.g., to maintain or prolong engraftment or colonization of the probiotic. In certain embodiments, at least or about 5 g, 10 g, 15 g, 18 g, 20 g, or 22 g of a mixture of one or more of 2′-fucosyllactose, 3′-sialyllactose, 6′-sialyllactose, lacto-N-tetraose, lacto-N-neotetraose, and difucosyllactose are administered at least once daily for at least 7, 10, 14, 21, 28, or 35 days.

### B.) Treatment Regimens

In certain embodiments, provided herein are methods for treating, preventing, or reducing the severity, risk, or likelihood of a disease, disorder, or condition, such as any described herein, e.g., in Section III. In particular embodiments, the provided methods are or include a treatment regimen. In certain embodiments, the treatment regimen is or includes one or more treatment phases where different combinations of one or more of the probiotic strain, e.g., B. longum subsp. infantis, a concentrated human milk permeate composition, and one or more synthetic oligosaccharides, e.g., human milk oligosaccharides, are administered. In certain aspects, steps, methods, or treatment phases that prolong or extend engraftment of the administered probiotic strain, e.g., B. longum subsp. infantis, augment, improve, and/or increase the efficacy of the treatment or prevention of the condition, disease, or disorder.

In some embodiments, the subject receives a medicament for the disease, disorder, or condition, e.g., any of those described herein such as in Section-III, and/or to reduce the likelihood or severity of one or more additional symptoms or complications of or resulting from the disease, disorder, or condition. In some embodiments, the medicament may be or include antibiotics. In certain embodiments, the treatment regimen begins after the final dose of the medicament, e.g., the antibiotic, has been administered. In certain embodiments, the treatment regimen begins while the medicament, e.g., antibiotics, are administered.

In particular embodiments, the treatment regimen has more than one treatment phase. In some aspects, treatment phases may differ and/or be distinguished by the presence, absence, dosages, and/or timing for administration of one or more of the at least one probiotic strain, the concentrated human milk permeate composition, and the one or more synthetic oligosaccharides. In certain embodiments, the treatment regimen contains one or more treatment phases that are or include a colonization phase, wherein the at least one probiotic strain, e.g., B. longum subsp. infantis, is administered. In some embodiments, a prebiotic, e.g., the concentrated human milk permeate composition and/or the one or more synthetic oligosaccharides, is administered in addition to the at least one probiotic strains during the colonization phase. In some embodiments, the first or initial treatment phase occurring within a treatment regimen is a colonization phase.

In some embodiments, the at least one probiotic strain, e.g., B. longum subsp. infantis, and a prebiotic is administered during a colonization phase to promote or establish engraftment or colonization of the at least one probiotic strain within the subject's gut and/or intestinal microbiome. In particular aspects, the colonization phase may serve as a portion of a treatment regimen and/or a method of treatment that results in an increase in the level or amount of the at least one probiotic strain within the subject's gut or intestinal microbiome. In certain embodiments, the at least one probiotic strain is exogenous to the subject's gut or intestinal microbiome, and the colonization phase may serve as a portion of a treatment regimen and/or method of treatment that results in the detection, identification, or measurement of the at least one probiotic strain within the subject's gut or intestinal microbiome. In some embodiments, the at least one probiotic strain is not detectable within the subject's gut or intestinal microbiome at the beginning of the colonization phase but is detectable within the subject's gut or intestinal microbiome during and/or by the end of the colonization phase, e.g., on a day when the at least one probiotic strain has not been administered.

In certain embodiments, the concentrated human milk permeate composition and the at least one probiotic strain are administered at least once to the subject, e.g., during a colonization phase and/or during a first or initial treatment phase of the treatment regimen. In some embodiments, the at least one probiotic strain is administered multiple times to the subject during the treatment phase, e.g., the colonization phase. In certain embodiments, the at least one probiotic strain is administered once, twice, three times, four times, five times, or more than five times per month; once, twice, three times, four times, five times, six times, seven times, or more than seven times per week; or once, twice, or more than twice daily during the treatment phase, e.g., the colonization phase. In some embodiments, the at least one probiotic strain is administered at least once per day during the colonization phase. In certain embodiments, the concentrated human milk permeate composition is administered at least once per day during the colonization phase. In some embodiments, the colonization phase is a probiotic treatment phase.

In particular embodiments, the at least one probiotic strain is administered at least once every two days or daily for at least 2, 3, 4, 5, 7, 10, 14, 21, or 28 days, e.g., consecutive days, during a treatment phase, e.g., a first or initial treatment phase and/or a colonization phase. In some embodiments, the at least one probiotic strain is administered in an amount of at least 1×10³, 1×10⁴, 1×10⁵, 1×10⁶, 5×10⁶, 1×10⁷, 1×10⁷, 5×10⁷, 1×10⁸, or 5×10⁸, 1×10⁹, 5×10⁹, 8×10⁹, 1×10¹⁰, 5×10¹⁰, or 1×10¹¹ colony forming units (CFU) per dose or per day during a treatment phase, e.g., a first or initial treatment phase and/or a colonization phase. In some embodiments, the at least one probiotic strain is administered in an amount of, of about, or at least 5×10⁶ colony forming units (CFU) per dose or per day during a treatment phase, e.g., a first or initial treatment phase and/or a colonization phase. In some embodiments, the at least one probiotic strain is administered in an amount of, of about, or at least 1×10⁸ colony forming units (CFU) per dose or per day during a first or initial treatment phase and/or a colonization phase. In some embodiments, the at least one probiotic strain is administered in an amount from 1×10⁸ to 1×10¹⁰ colony forming units (CFU) per dose or per day during a first or initial treatment phase and/or a colonization phase.

In particular embodiments, a prebiotic is administered in addition to the at least one probiotic strain during a treatment phase, e.g., a first or initial treatment phase and/or a colonization phase. In particular embodiments, the prebiotic is a concentrated human milk permeate composition. In certain embodiments, the concentrated human milk permeate composition is administered at least once every two days or daily for at least 2, 3, 4, 5, 7, 10, 14, 21, or 28 days, e.g., consecutive days, in addition to the at least one probiotic strain during the treatment phase, e.g., the first or initial treatment phase and/or the colonization phase. In certain embodiments, the concentrated human milk permeate composition is administered in an amount of about or of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day, e.g., by total weight of the human milk oligosaccharides of the composition, in addition to the at least one probiotic strain during the treatment phase, e.g., the first or initial treatment phase and/or the colonization phase. In some embodiments, the concentrated human milk permeate composition is administered in an amount of from 0.1 g to 50 g; 0.5 g to 25 g, 1 g to 20 g, 2 g to 18 g, 1 g to 5 g, 2 g to 3 g, 3 g to 6 g, 4 g to 5 g, 5 g to 10 g, 8 g to 10 g, 10 g to 20 g, 15 g to 20 g, 17 g to 19 g, or 20 g to 25 g total human milk oligosaccharides per day in addition to the at least one probiotic strain during the treatment phase, e.g., the first or initial treatment phase and/or the colonization phase. In some embodiments, the concentrated human milk permeate composition is administered in an amount of, of about, or of at least 9 g, 10 g, 12 g g, 15 g, 18 g, 20 g, 22 g, or 25 g total human milk oligosaccharides per day in addition to the at least one probiotic strain during a treatment phase, e.g., the first or initial treatment phase and/or the colonization phase.

In some embodiments, the concentrated human milk permeate composition is administered in addition to the at least one probiotic strain during a portion of a colonization phase. In certain embodiments, the concentrated human milk permeate composition is administered at least once every two days or daily for the first or initial 2, 3, 4, 5, 7, 10, or 14 days of the colonization phase in addition to the at least one probiotic strain. In particular embodiments, no additional prebiotics are administered after treatment with the concentrated human milk permeate has been completed, ceased, or ended. In some embodiments, one or more synthetic oligosaccharides, e.g., synthetic human milk oligosaccharides, are administered after treatment with the concentrated human milk permeate has been completed, ceased, or ended. In particular embodiments, the one or more synthetic oligosaccharides are administered at least once every two days or at least once daily for the remainder of the colonization phase. In certain embodiments, the one or more synthetic oligosaccharides, e.g., synthetic human milk oligosaccharides, are administered in an amount of from 0.1 g to 50 g; 0.5 g to 25 g, 1 g to 20 g, 2 g to 18 g, 1 g to 5 g, 2 g to 3 g, 3 g to 6 g, 4 g to 5 g, 5 g to 10 g, 8 g to 10 g, 10 g to 20 g, 15 g to 20 g, 17 g to 19 g, or 20 g to 25 g per day in addition to the at least one probiotic strain for the remainder of the colonization phase. In certain embodiments, the one or more synthetic oligosaccharides, e.g., synthetic human milk oligosaccharides, are administered in an amount of, of about, or of at least 9 g, 10 g, 12 g, 15 g, 18 g, 20 g, 22 g, or 25 g per day in addition to the at least one probiotic strain during the remainder of the colonization phase.

In certain embodiments, the one or more synthetic oligosaccharides, e.g., human milk oligosaccharides, and the concentrated human milk permeate composition are administered during the colonization phase, e.g., in addition to the at least one probiotic strain. In certain embodiments, the one or more synthetic oligosaccharides and the concentrated human milk permeate composition are administered in alternating doses or on alternating days. In particular embodiments, the synthetic oligosaccharides are administered every day except for every two, three, four, five, seven, or fourteen days or more during the colonization phase, when the concentrated human milk permeate composition is administered. In some embodiments, the one or more synthetic oligosaccharides, e.g., human milk oligosaccharides, are administered every day except for every two, three, four, five, seven, or fourteen days or more, in an amount of at least 2 g, 4 g, 4.5 g, 5 g, 9 g, 10 g, 15 g, 18 g, 20 g, 22 g, 25 g, or 50 g per day, e.g., by total weight of the human milk oligosaccharides during the colonization phase. In particular embodiments, the one or more synthetic oligosaccharides, e.g., human milk oligosaccharides, are administered in an amount of 10 g to 25 g total human milk oligosaccharides per day. In some embodiments, on alternate days or every two, three, four, five, seven, or fourteen days or more, the concentrated human milk permeate composition is administered in an amount of at least 2 g, 4 g, 4.5 g, 5 g, 9 g, 10 g, 15 g, 18 g, 20 g, 22 g, 25 g, or 50 g per day, e.g., by total weight of the human milk oligosaccharides during the colonization phase. In particular embodiments, the concentrated human milk permeate composition is administered in an amount of 10 g to 25 g total human milk oligosaccharides per day.

In some embodiments, the treatment regimen includes treatment phases where the at least probiotic strain, e.g., B. longum subsp. infantis, is not administered. In certain embodiments, the treatment regimen includes one or more treatment phases where a prebiotic, e.g., the concentrated human milk permeate composition and/or the one or more synthetic oligosaccharides, but not the at least one probiotic strain are administered. In certain aspects, such administration of the prebiotic in the absence of and/or without administration at least the one probiotic strain to a subject may serve to maintain, prolong, or extend the engraftment or colonization of a probiotic strain when the probiotic strain was previously administered to the subject. Thus, in some aspects, a treatment phase where a prebiotic, but not the at least one probiotic strain, is administered occurs subsequent to and/or after colonization phase. Treatment phases that include administration of a prebiotic but not administration of the at least one probiotic may be referred to herein as a “prebiotic treatment phase.”

In particular embodiments, the provided methods of treatment and/or treatment regimens are or include multiple treatment phases that include at least one maintenance phase. In particular embodiments, a maintenance phase is a treatment phase that occurs after a treatment phase that is or includes administration of the at least one probiotic strain and/or after a colonization phase. In some embodiments, the maintenance phase is or includes a treatment phase wherein one or more prebiotics are administered, e.g., to maintain or prolong engraftment and/or colonization of the at least one probiotic strain. In certain embodiments, the at least one probiotic strain is detectable within the subject's gut and/or intestinal microbiome throughout the duration of the maintenance phase, e.g., such as including on days where the at least one probiotic strain has not been administered. In particular embodiments, the at least one probiotic strain is detectable within the subject's gut and/or intestinal microbiome at the beginning, e.g., on the first day, of the maintenance phase. In certain embodiments, the at least one probiotic strain is detectable at the end, e.g., on the last day, of the maintenance phase.

In particular embodiments, the at least one probiotic strain is not administered during a maintenance phase. In some embodiments, the prebiotic, e.g., concentrated human milk permeate composition and/or the one or more synthetic human milk oligosaccharides, is administered at least once every two days or daily for at least 2, 3, 4, 5, 7, 10, 14, 21, or 28 days, e.g., consecutive days when the at least one probiotic strain is not administered, such as during a treatment phase, e.g., during a subsequent treatment phase occurring after a colonization phase and/or a maintenance phase. In some embodiments, the maintenance phase is a prebiotic phase.

In certain embodiments, the at least one probiotic strain is administered with less frequency and/or in lower doses or amounts than the frequency, doses, and/or amounts of a colonization phase. In certain embodiments, the at least one probiotic strain may be administered during a maintenance phase, e.g., at least once, twice, three times, five times, or more. In certain embodiments, the at least one probiotic strain may be administered for several consecutive days, e.g., at least 1, 2, 3, 4, or 5 days at the beginning of the maintenance phase. In certain embodiments, the at least one probiotic strain is administered at least once every 4 weeks, once every 2 weeks, once every week, or at least once every three days or every two days during a maintenance phase.

In certain embodiments, the one or more synthetic oligosaccharides, e.g., synthetic human milk oligosaccharides, are administered in an amount of about or of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day during a treatment phase, e.g., a maintenance phase and/or a treatment phase subsequent to a colonization phase. In some embodiments, the one or more synthetic oligosaccharides, e.g., synthetic human milk oligosaccharides, are administered in an amount of about or of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 22 g, 25 g, or 50 g per day during a treatment phase, e.g., a maintenance phase and/or a treatment phase subsequent to a colonization phase. In some embodiments, the one or more synthetic oligosaccharides, e.g., synthetic human milk oligosaccharides, are administered in an amount of from 0.1 g to 50 g; 0.5 g to 25 g, 1 g to 20 g, 2 g to 18 g, 1 g to 5 g, 2 g to 3 g, 3 g to 6 g, 4 g to 5 g, 5 g to 10 g, 8 g to 10 g, 10 g to 20 g, 15 g to 20 g, 17 g to 19 g, or 20 g to 25 g per day during a treatment phase e.g., a maintenance phase and/or a treatment phase subsequent to a colonization phase. In particular embodiments, the one or more synthetic oligosaccharides, e.g., synthetic human milk oligosaccharides, are administered in an amount of, of about, or of at least at least 9 g, 10 g, 12 g, 15 g, 18 g, 20 g, 22 g, or 25 g per day during a treatment phase, e.g., a maintenance phase and/or a treatment phase subsequent to a colonization phase.

In particular embodiments, the concentrated human milk permeate composition is administered during a maintenance phase. In some aspects, a maintenance phase where the concentrated human milk permeate is administered takes place between (i) a colonization phase wherein the at least one probiotic strain and the concentrated human milk permeate composition are administered and (ii) a maintenance phase wherein the one or more synthetic oligosaccharides, e.g., synthetic human milk oligosaccharides, are administered. In certain embodiments, the concentrated human milk permeate composition is administered during the maintenance phase at the same doses or amounts and/or the same frequency as during the colonization phase.

In certain embodiments, the concentrated human milk permeate composition is administered at least once every two days or daily for at least 2, 3, 4, 5, 7, 10, 14, 21, or 28 days, e.g., consecutive days, during a treatment phase, e.g., a maintenance phase and/or a treatment phase subsequent to a colonization phase. In some embodiments, the concentrated human milk permeate composition is administered in an amount of about or of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day, e.g., by total weight of the human milk oligosaccharides of the composition during a treatment phase, e.g., a maintenance phase and/or a treatment phase subsequent to a colonization phase. In some embodiments, the concentrated human milk permeate composition is administered in an amount of about or of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 22 g, 25 g, or 50 g total human milk oligosaccharides per day during a treatment phase, e.g., a maintenance phase and/or a treatment phase subsequent to a colonization phase. In some embodiments, the concentrated human milk permeate composition is administered in an amount of from 0.1 g to 50 g; 0.5 g to 25 g, 1 g to 20 g, 2 g to 18 g, 1 g to 5 g, 2 g to 3 g, 3 g to 6 g, 4 g to 5 g, 5 g to 10 g, 8 g to 10 g, 10 g to 20 g, 15 g to 20 g, 17 g to 19 g, or 20 g to 25 g total human milk oligosaccharides per day during a treatment phase, e.g., a maintenance phase and/or a treatment phase subsequent to a colonization phase. In certain embodiments, the concentrated human milk permeate composition is administered in an amount of, of about, or of at least 9 g, 10 g, 12 g, 15 g, 18 g, 20 g, 22 g, or 25 g total human milk oligosaccharides per day during a treatment phase, e.g., a maintenance phase and/or a treatment phase subsequent to a colonization phase.

In some embodiments, the one or more synthetic oligosaccharides, e.g., human milk oligosaccharides, and the concentrated human milk permeate composition are administered during the maintenance phase. In particular embodiments, the one or more synthetic oligosaccharides and the concentrated human milk permeate composition are administered in alternating doses or on alternating days. In certain embodiments, the synthetic oligosaccharides are administered every day except for every two, three, four, five, seven, or fourteen or more days during the colonization phase, when the concentrated human milk permeate composition is administered. In some embodiments, the one or more synthetic oligosaccharides, e.g., human milk oligosaccharides, are administered every day except for every two, three, four, five, seven, or fourteen days or more, in an amount of at least 2 g, 4 g, 4.5 g, 5 g, 9 g, 10 g, 15 g, 18 g, 20 g, 22 g, 25 g, or 50 g per day, e.g., by total weight of the human milk oligosaccharides during the maintenance phase. In certain embodiments, the one or more synthetic oligosaccharides, e.g., human milk oligosaccharides, are administered in an amount of 10 g to 25 g total human milk oligosaccharides per day. In particular embodiments, on alternate days or every two, three, four, five, seven, or fourteen days or more, the concentrated human milk permeate composition is administered in an amount of at least 2 g, 4 g, 4.5 g, 5 g, 9 g, 10 g, 15 g, 18 g, 20 g, 22 g, 25 g, or 50 g per day, e.g., by total weight of the human milk oligosaccharides during the maintenance phase. In certain embodiments, the concentrated human milk permeate composition is administered in an amount of 10 g to 25 g total human milk oligosaccharides per day.

In some embodiments, a treatment phase may last for a duration of time that is at least 1 day, 2 days, 3 days, 4 days, 5 days, 6 days, 7 days, 8 days, 9 days, 10 days, 1 week, 2 weeks, 3 weeks, 4 weeks, 6 weeks, 8 weeks, 1 month, 2 months, or 3 months in length. In some embodiments, the at least one probiotic strain and/or the prebiotics, e.g., the concentrated human milk permeate composition and/or the one or more synthetic oligosaccharides, are administered at least once, twice, or at least three, four, five, seven, ten, fourteen, or twenty-one times during a treatment phase. In certain embodiments, the at least one probiotic strain and/or the prebiotics, e.g., the concentrated human milk permeate composition and/or the one or more synthetic oligosaccharides, are administered at least once or twice weekly, once every other day, or at least once or twice a day during the treatment phase. In some embodiments, a subsequent treatment phase begins immediately after the earlier treatment phase is completed. In particular embodiments, the subsequent treatment phase begins after a delay after the earlier treatment regimen is completed. In some embodiments, the delay is at least 1 day, 2 days, 3 days, 4 days, 5 days, 6 days, 7 days, 8 days, 9 days, 10 days, 1 week, 2 weeks, 3 weeks, 4 weeks, 6 weeks, 8 weeks, 1 month, 2 months, or 3 months in length.

In some embodiments, the treatment phases, e.g., a colonization phase and a subsequent maintenance phase, may be cycled or repeated, either in a full or truncated form, during the treatment regimen. In some embodiments, the provided methods may repeat a treatment regimen in multiple cycles, for example, a subject may complete a treatment regimen, and after a pause in treatment, the regimen may be performed again. In some embodiments, a subsequent treatment regimen begins immediately after the earlier regimen is completed. In some embodiments, the subsequent treatment regimen begins after a delay after the earlier treatment regimen is completed. In certain embodiments, the delay is at least 1 day, 2 days, 3 days, 4 days, 5 days, 6 days, 7 days, 8 days, 9 days, 10 days, 1 week, 2 weeks, 3 weeks, 4 weeks, 6 weeks, 8 weeks, 1 month, 2 months, or 3 months in length.

### C.) Exemplary Treatment Regimens

In some embodiments, provided herein are methods of administering prebiotics, e.g., the concentrated human milk permeate composition and/or one or more synthetic oligosaccharides, and at least one probiotic, e.g., a strain of Bifidobacterium such as B. longum subsp. infantis, to treat or prevent a disease, disorder, or condition associated with one or more of inflammation, infection, allergy, immune dysfunction, or dysbiosis of the intestinal microbiome in a subject in need thereof. In certain embodiments, the probiotic strain is capable of consuming (e.g., hydrolyzing) the prebiotics. In particular embodiments, the probiotic strain is capable of internalizing and consuming (e.g., hydrolyzing) the prebiotics. In various embodiments, the probiotic strain is capable of internalizing and consuming (e.g., hydrolyzing) human milk oligosaccharides, e.g., the human milk oligosaccharides of the concentrated human milk permeate composition and the one or more synthetic human milk oligosaccharides. In particular embodiments, a probiotic strain that is capable of consuming, internalizing, and/or hydrolyzing a prebiotic is capable of consuming, internalizing, and/or hydrolyzing the prebiotic in vivo such as within the human gut.

In some embodiments, the treatment regimen is or includes multiple treatment phases that are or include at least one colonization phase and one or more subsequent maintenance phases. In certain embodiments, the treatment regimen includes a colonization phase wherein the at least one probiotic strain, e.g., B. longum subsp. infantis, is administered and a maintenance phase that occurs after the colonization phase. In some embodiments, the treatment regimen includes (i) a colonization phase wherein the at least one probiotic strain and the concentrated human milk permeate composition is administered and (ii) one or more maintenance phases that occur subsequent to or after the colonization phase. In particular embodiments, the one or more synthetic oligosaccharides, e.g., synthetic human milk oligosaccharides, are administered during one or more of the maintenance phases. In particular embodiments, the treatment regimen includes (i) a colonization phase wherein the at least one probiotic strain and the concentrated human milk permeate composition is administered, and (ii) a maintenance phase wherein the one or more synthetic oligosaccharides, e.g., synthetic human milk oligosaccharides, are administered. In particular embodiments, the treatment regimen includes (i) a colonization phase wherein the at least one probiotic strain and the concentrated human milk permeate composition is administered, (ii) a maintenance phase wherein the concentrated human milk permeate composition is administered, and (iii) a maintenance phase wherein the one or more synthetic oligosaccharides, e.g., synthetic human milk oligosaccharides, are administered.

In certain embodiments, the treatment regimen is or includes one or more treatment phases that include a first or initial treatment phase that is a colonization phase and one or more subsequent treatment phases that are or include one or more maintenance phases. In certain embodiments, the colonization phase has a duration of and/or lasts for at least 3 days, 5 days, 7 days, 10 days, 1 week, 2 weeks, 3 weeks, or 4 weeks. In particular embodiments, the at least one probiotic strain is B. longum subsp. infantis. In certain embodiments, B. longum subsp. infantis is administered in a dose of at least 1×10⁸ CFU at least once every two days or at least once daily during the colonization phase. In some embodiments, doses of the concentrated human milk permeate composition from 10 g to 25 g by weight of total human milk oligosaccharides are administered at least once every two days or at least once daily during the colonization phase. In some embodiments, the treatment regimen has one or more maintenance phases that are subsequent to the colonization phase. In particular embodiments, the maintenance phase lasts for at least 3 days, 5 days, 7 days, 10 days, 1 week, 2 weeks, 3 weeks, or 4 weeks. In certain embodiments, the at least one maintenance phase is or includes a maintenance phase that includes administering one or more synthetic human milk oligosaccharides. In particular embodiments, a dose from 10 g to 25 g of the synthetic human milk oligosaccharides are administered at least once every other day or at least once daily during a maintenance phase. In certain embodiments, the synthetic human milk oligosaccharides include one or more of one or more of 2′-fucosyllactose, 3-fucosyllactose, lacto-N-tetraose, or lacto-N-neotetraose. In certain embodiments, the one or more synthetic human milk oligosaccharides are or include 2′-fucosyllactose, 3′-sialyllactose, 6′-sialyllactose, lacto-N-tetraose, lacto-N-neotetraose, and difucosyllactose. Colonization and maintenance phases may be performed once during a treatment regiment, or, in some embodiments, may be repeated or cycled through multiple times during a treatment regimen. In certain embodiments, treatment phases may be truncated when they are repeated in a treatment regimen, e.g., relative to when they are first performed during the treatment regimen.

In some embodiments, the treatment regimen is or includes multiple treatment phases that include at least a first or initial treatment phase that is a colonization phase and at least two subsequent treatment phases that are maintenance phases. In certain embodiments, the colonization phase lasts for at least 3 days, 5 days, 7 days, 10 days, 1 week, 2 weeks, 3 weeks, or 4 weeks. In some embodiments, the at least one probiotic strain is B. longum subsp. infantis and is administered in a dose of at least 1×10⁸ CFU at least once every two days or at least once daily during the colonization phase. In some embodiments, doses of the concentrated human milk permeate composition from 4.5 g to 25 g or 15 g to 25 g by weight of total human milk oligosaccharides are administered at least once every two days or at least once daily during the colonization phase. In some embodiments, the treatment regimen has a second treatment phase that is a maintenance phase that lasts for at least 3 days, 5 days, 7 days, 10 days, 1 week, 2 weeks, 3 weeks, or 4 weeks, wherein the concentrated human milk permeate is administered at a dose from 4.5 g to 25 g or 10 g to 25 g by weight of total human milk oligosaccharides at least once every other day or at least once daily. In some embodiments, the treatment regimen has a third treatment phase that is a maintenance phase that lasts for at least 3 days, 5 days, 7 days, 10 days, 1 week, 2 weeks, 3 weeks, or 4 weeks, wherein the one or more synthetic human milk oligosaccharides are administered in an amount from 4.5 g to 25 g or 15 g to 25 g per day every other day or every day during the treatment phase. In some embodiments, the one or more synthetic human milk oligosaccharides are or include 2′-fucosyllactose, 3′-sialyllactose, 6′-sialyllactose, lacto-N-tetraose, lacto-N-neotetraose, and difucosyllactose. In certain embodiments, the synthetic human milk oligosaccharides include one or more of one or more of 2′-fucosyllactose, 3-fucosyllactose, lacto-N-tetraose, or lacto-N-neotetraose. The treatment phases, e.g., colonization or maintenance phases, may be performed once during a treatment regimen, or in some embodiments they may be repeated or cycled multiple times during a treatment regimen. In certain embodiments, treatment phases may be truncated when they are repeated in a treatment regimen, e.g., relative to when they are first performed during the treatment regimen.

In particular embodiments, the subject has received or will receive a transplant, e.g., a solid organ transplant or an allogenic hematopoietic stem cell transplant. In certain embodiments, the treatment regimen begins at least 3 days, 5 days, 7 days, or 14 days prior to the transplant. In certain embodiments, the colonization phase begins at least 3 days, 5 days, 7 days, or 14 days prior to the transplant. In particular embodiments, the treatment colonization phase begins after the transplant, such as within 14 days, 10 days, 7 days, 5 days, 3 days, 1 day, or immediately after the transplant. In certain embodiments, the colonization phase begins prior to the transplant and persists or lasts until at least 3 days, 5 days, 7 days, 10 days, or 14 days after the transplant. In particular embodiments, the subject undergoes treatment with antibiotics prior to, during, and/or after the transplant. In some embodiments, the subject undergoes treatment with antibiotics beginning at least 3, 5, 7, or 14 days prior to the transplant. In certain embodiments, the treatment with antibiotics persists or lasts until at least 3 days, 5 days, 7 days, 10 days, or 14 days after the transplant. In particular embodiments, the treatment regimen begins within the last 7 days, 5 days, 3 days, or 1 day of the antibiotic treatment. In some embodiments, the treatment regimen begins immediately after the antibiotic treatment is ceased, ended, or completed. In certain embodiments, the colonization phase begins during the treatment with the antibiotics and persists until at least 3 days, 5 days, 7 days, 10 days, or 14 days after the antibiotic treatment is ceased, ended, or completed.

In some embodiments, the antibiotic treatment is or includes administration of of a cephalosporin, e.g., a fourth-generation cephalosporin such as cefpirome or cefepime. In certain embodiments, the antibiotic treatment is or includes administration of a glycopeptide antibiotic, such as one or more of vancomycin, teicoplanin, telavancin, ramoplanin and decaplanin, corbomycin, complestatin, or bleomycin). In certain embodiments, the antibiotic treatment is or includes administration of a beta-lactamase inhibitor, e.g., piperacillin-tazobactam. In particular embodiments, the antibiotic treatment is or includes a treatment with a carbapenem, e.g., one or more of doripenem, ertapenem, imipenem, or meropenem. In some embodiments, the antibiotic treatment is or includes administration of an aminoglycoside, such as one or more of paromomycin, amikacin, plazomicin, tobramycin, neomycin, kanamycin, gentamicin, or amikacin lipsome. In certain embodiments, the antibiotic treatment is or includes administration of a quinolone antibiotic, such as one or more of ciprofloxacin, delafloxacin, gemifloxacin, levofloxacin, moxifloxacin, or ofloxacin. In particular embodiments, the antibiotic treatment is or includes administration of one or more of vancomycin, polymyxin B, metronidazole, ciprofloxacin. In certain embodiments, the antibiotic treatment is or includes administration of rifaximin.

In particular embodiments, the treatment regimen includes a first treatment phase that is a colonization phase and at least one subsequent treatment phase that is a maintenance phase. In certain embodiments, the colonization phase has a duration of at least 9 days, 10 days, or 14 days. In some embodiments, B. longum subsp. infantis is administered at a dose of at least at least 1×10⁸ CFU per day, and the concentrated human milk permeate composition is administered at a dose from 10 g to 25 g total human milk oligosaccharides by weight per day, daily throughout the colonization phase. In some embodiments, the maintenance phase has a duration of at least 14 days. In certain embodiments, one or more synthetic human milk oligosaccharides are administered daily at a dose from 10 g to 25 g total human milk oligosaccharides per day during the maintenance phase. In some embodiments, the one or more synthetic human milk oligosaccharides are or include one or more of 2′-fucosyllactose, 3-fucosyllactose, 3′-sialyllactose, 6′-sialyllactose, lacto-N-tetraose, lacto-N-neotetraose, or difucosyllactose. In particular embodiments, the one or more synthetic human milk oligosaccharides are or include one or more of 2′-fucosyllactose, 3-fucosyllactose, lacto-N-tetraose, or lacto-N-neotetraose. In some embodiments, the one or more synthetic human milk oligosaccharides are 2′-fucosyllactose and lacto-N-neotetraose.

Particular embodiments contemplate that the effectiveness of the provided methods relate, at least in part, to interactions, e.g., synergistic interactions, among the different compositions. For example, in some aspects, the provided methods successfully treat, correct, or ameliorate dysbiosis and related disorders to a much greater extent than what would be expected from an alternative treatment, e.g., alternative medicaments, alternative probiotics, or alternative prebiotics, or from treatment with the prebiotics or the probiotic strain alone. In certain embodiments, administration of a probiotic strain of B. longum subsp. infantis and the prebiotics successfully treat, correct, or ameliorate dysbiosis and related disorders to a much greater extent than what would be expected from treatment with an alternative treatment, e.g., alternative medicaments, alternative probiotics, or alternative prebiotics, or from treatment with B. longum subsp. infantis or prebiotics alone.

### II. COMPOSITIONS, KITS, AND ARTICLES OF MANUFACTURE

Provided herein are compositions, kits, and articles of manufacture that are or include at least one strain of probiotic bacterium (also referred to herein as a probiotic strain of bacteria, a probiotic strain, or a probiotic) and one or more prebiotic compositions. In certain embodiments, the prebiotics are or include a concentrated human milk permeate composition that contains human milk oligosaccharides, e.g., a plurality of at least 10, 25, 50, or more human milk oligosaccharides. In particular embodiments, the prebiotics are or include one or more synthetic oligosaccharides, e.g., one or more human milk oligosaccharides that are synthesized or produced from a non-human milk source. In some embodiments, the provided compositions, kits, and article of manufacture are or include both the at least one probiotic strain and one or more prebiotics.

In certain embodiments, the at least one probiotic strain and the prebiotics are included in separate compositions, e.g., are administered separately to a subject. Thus, in certain embodiments, provided herein are kits and articles of manufacture that include both of (i) a composition that is or includes at least one probiotic strain and (ii) a composition that is or includes prebiotics, e.g., a concentrated human milk permeate composition and/or one or more synthetic oligosaccharides. Also provided are kits and articles of manufacture that are or include one or more compositions that each contain both the at least one probiotic strain and the prebiotics.

In certain embodiments, the provided compositions, kits, and articles of manufacture contain or include any of the concentrated human milk permeate compositions that are described herein, such as in Section II-A. In some embodiments, the provided compositions, kits, and articles of manufacture contain or include one or more synthetic oligosaccharides, e.g., synthetic human milk oligosaccharides, that are described herein, such as in Section II-B. In particular embodiments, the provided compositions, kits, and articles of manufacture contain or include any of the probiotic strains, e.g., of Bifidobacterium, described herein, such as those described in Section II-C. In some aspects, the provided kits and articles of manufacture may also include labels or instructions for use. In some embodiments, such labels or instructions for use may describe any of the uses or methods provided herein, such as those described in Section I.

In some embodiments, the at least one probiotic strain is capable of internalizing the prebiotics, e.g., the concentrated human milk permeate composition and/or synthetic oligosaccharides. In some embodiments, the prebiotics are formulated to promote the growth or expansion of the at least one probiotic strain in vivo, e.g., in the gut of a human. In certain embodiments the prebiotics selectively or exclusively serve as a carbon source for the at least one probiotic strain. In some embodiments, human milk oligosaccharides selectively or exclusively serve as an energy source for the probiotic strain(s).

Various embodiments contemplate that the administration of the prebiotics, e.g., the concentrated human milk permeate compositions and/or synthetic oligosaccharides, and the one or more probiotic strains synergistically prevent or reduce the likelihood, probability, or risk of a disease, disorder, or condition, e.g., an inflammatory or autoimmune related disease, disorder, or condition (such as any of those described herein, e.g., in Section III), in a subject to a greater degree than what would be expected based on the administration of either the one or more probiotic strains or the prebiotics alone.

In certain embodiments, administration of the prebiotics and the one or more probiotic strains synergistically prevent or reduce the likelihood, probability, or risk of dysbiosis, e.g., of the human intestinal microbiome, to a greater degree than what would be expected based on the administration of either the one or more probiotic strains or the prebiotics alone. In some embodiments, administration of the prebiotics and the one or more probiotic strains synergistically treat, reduce, or ameliorate dysbiosis, and/or one or more symptoms of a disease, disorder, or condition that may be associated with dysbiosis, to a greater degree than what would be expected based on the administration of either the one or more probiotic strains or the prebiotics alone. Particular embodiments contemplate that the degree of dysbiosis, as well as a reduction or decrease of dysbiosis, may be determined by those of skill in the art by routine methods, including but not limited to routine genetic techniques (e.g., 16S sequencing) to determine the presence, portion, or amount of different microbiota genera, species, and/or strains.

Particular embodiments contemplate that the administration of the prebiotics and the at least one probiotic strain synergistically prevent or reduce the likelihood, probability, or risk of graft versus host disease in a subject receiving a transplant, e.g., a bone marrow transplant or an allogeneic stem cell transplantation (allo-HSCT), to a greater degree than what would be expected based on the administration of either the one or more probiotic strains or the prebiotics alone. In particular embodiments, it is contemplated that the administration of the probiotic strain and the prebiotics synergistically reduces, ameliorates, treats, alleviates or prevents the severity of one or more symptoms associated with GVHD, e.g., to a greater degree than what would be expected based on the administration of either the probiotic strain or the prebiotics alone.

In some embodiments, a concentrated human milk permeate composition containing human milk oligosaccharides, one or more synthetic oligosaccharides, and at least one probiotic strain of bacterium capable of consuming human milk oligosaccharides are administered to a subject in need thereof. In certain embodiments, the human milk permeate composition and the at least one probiotic strain is administered to the subject, e.g., to establish or promote engraftment of the at least one probiotic strain within the subject's gut or intestinal microbiome, and then one or more synthetic oligosaccharides are administered, e.g., to maintain the presence or engraftment of the probiotic strain within the subject's gut or intestinal microbiome.

### A.) Concentrated Human Milk Permeate Compositions

In some embodiments, the prebiotic is or includes a concentrated human milk permeate composition, e.g., containing human milk oligosaccharides, that promotes the growth or expansion of the at least one probiotic strain, e.g., in vivo such as within the human gut and/or within the human intestinal microbiome. In certain embodiments, the concentrated human milk permeate composition, promotes, e.g., selectively or exclusively, the colonization, expansion, extension, engraftment, or increased presence of the at least one probiotic strain within the microbiome. In particular embodiments, the concentrated human milk permeate composition promotes the growth or expansion of a Bifidobacterium probiotic strain such as B. longum subsp. infantis, e.g., in vivo such as in the human gut. In certain embodiments, the concentrated human milk permeate composition contains a plurality of oligosaccharides, e.g., HMOs, that promote, e.g., selectively or exclusively, the colonization, expansion, extension, or increased presence of one or more strains of Bifidobacterium, e.g., B. longum subsp. infantis, within the microbiome.

In particular embodiments, the concentrated human milk permeate composition contains a plurality of human milk oligosaccharides. In particular embodiments, the concentrated human milk permeate composition is obtained, derived, or procured by a method described herein such as in Section II-A-(i).

In some embodiments, the concentrated human milk permeate composition is or includes human milk oligosaccharides. In some embodiments, the concentrated human milk permeate composition is or includes human milk oligosaccharides capable of being internalized by one or more strain of Bifidobacterium such as a strain of B. longum subsp. infantis.

In some embodiments, all or a portion of the oligosaccharides of the concentrated human milk permeate composition are human milk oligosaccharides, e.g., at least 25%, 50%, 75%, 90%, 95%, or 99% of the oligosaccharides by either i) the percentage of oligosaccharide species present in the composition or ii) by total weight of the oligosaccharides in the composition. In certain embodiments, all or essentially all of the oligosaccharides of concentrated human milk permeate composition are human milk oligosaccharides.

In some aspects, the human milk oligosaccharides are oligosaccharides that are present or found in human milk. In certain aspects, all HMOs are composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of sialic acid. In certain aspects, HMO biosynthesis appears to follow a basic blueprint: all HMOs contain lactose (Galβ1-4Glc) at their reducing end, which can be elongated by the addition of β1-3- or β1-6-linked lacto-N-biose (Galβ1-3GlcNAc-, type 1 chain) or N-acetyllactosamine (Galβ1-4GlcNAc-, type 2 chain). Elongation with lacto-N-biose appears to terminate the chain, whereas N-acetyllactosamine can be further extended by the addition of one of the two disaccharides. A 01-6 linkage between two disaccharide units introduces chain branching. Branched structures are designated as iso-HMO; linear structures without branches as para-HMO. Lactose or the elongated oligosaccharide chain can be fucosylated in α1-2, α1-3, or α1-4 linkage and/or sialylated in α2-3 or α2-6 linkage. Particular embodiments contemplate that HMOs structures are known and identifiable, and are described, e.g., in Bode, Glycobiology (2012) 22(9): 1147-1162; Prudden et al. PNAS (2017) 114(27): 6954-6959; Kobata, Pro. Jpn. Acad., Ser B (2010) 86:731-747; and Smilowitz et al Annu Rev Nutr. (2014) 34: 143-169.

In some embodiments, the concentrated human milk permeate composition is not human milk (e.g., breastmilk or whole human milk). In certain embodiments, the concentrated human milk permeate composition may be derived from or obtained from human milk, such as with one or more steps to separate or remove macronutrients, e.g., fat, protein, and/or carbohydrates, while retaining human milk oligosaccharides. In particular embodiments, the concentrated human milk permeate composition is not a human milk fortifier. In certain embodiments, the concentrated human milk permeate composition has less than 2 g per 100 mL of protein and/or has less than 3 g per 100 mL of fat). In various embodiments, the concentrated human milk permeate composition is or includes less than 2%, 1.5%, 1%, 0.5%, or 0.1% protein (by weight/volume or w/v). In particular embodiments, the concentrated human milk permeate composition is or includes less than 3%, 2.5%, 2%, 1.5%, 1%, 0.5%, or 0.1% fat (w/v).

In particular embodiments, the concentrated human milk permeate composition is or contains a plurality of human milk oligosaccharides. In some embodiments, the concentrated human milk permeate composition is or includes a plurality of, of about, or of at least 2, 3, 5, 10, 25, 50, 75, 100, 125, 150 different individual human milk oligosaccharides, e.g., human milk oligosaccharides with different individual chemical formulas or chemical structures. In certain embodiments, the concentrated human milk permeate composition is or includes a plurality of, of about, or of at least 10, 25, 50, 75, 100, 125, 150 different individual human milk oligosaccharides. In some embodiments, the concentrated human milk permeate composition is or includes a plurality of, of about, or of at least 25 different individual HMOs. In some embodiments, the concentrated human milk permeate composition is or includes a plurality of, of about, or of at least 80 different individual HMOs. Particular embodiments contemplate that one of skill may determine if an oligosaccharide is an HMO, such as if the oligosaccharide has a chemical formula and structure that is identical to an oligosaccharide that is found in human milk. In particular embodiments, determination of whether an oligosaccharide is a human milk oligosaccharide may be performed as a matter of routine.

In some embodiments, the concentrated human milk permeate composition is or is obtained from an ultra-filtered permeate from human skim milk. In some embodiments, the concentrated human milk permeate composition is or is obtained from a process described herein, e.g., in Section-II-A-(i). In certain embodiments, the concentrated human milk permeate composition is similar or identical to those as described in U.S. Pat. No. 8,927,027 or in PCT Application No. WO 2018053535, incorporated herein by reference.

In some embodiments, the concentrated human milk permeate composition contains a plurality of, of about, or of at least 1, 2, 3, 5, 10, 25, 50, 75, 100, 125, or 150 different individual human milk oligosaccharides, e.g., human milk oligosaccharides with different individual chemical formulas or chemical structures. In some embodiments, the concentrated human milk permeate composition is or includes a plurality of, of about, or of at least 25 different individual HMOs. In some embodiments, the concentrated human milk permeate composition is or includes a plurality of, of about, or of at least 80 different individual HMOs.

In some embodiments, the concentrated human milk permeate composition includes some or all of 2′-fucosyllactose, 3-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose. In particular embodiments, the concentrated human milk permeate composition includes all of 2′-fucosyllactose, 3-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose.

In certain embodiments, the concentrated human milk permeate composition includes some or all of 2′-fucosyllactose, lacto-N-tetraorose, 3-sialyllactose, 3-fucosyllactose, lacto-N-fucopentaose I, lacto-N-fucopentaose II, and 6′sialyllactose. In particular embodiments, the concentrated human milk permeate composition includes some or all of 2′-fucosyllactose, 3-fucosyllactose, 3′-sialyllactose, 6′-sialyllactose, Lacto-N-tetraose, Lacto-N-neotetraose, Lacto-N-fucopentaose I, Lacto-N-fucopentaose II, Lacto-N-fucopentaose III, Sialyllacto-N-tetraose b, Sialyllacto-N-tetraose c, Lacto-N-difuco-hexaose I, Lacto-N-difuco-hexaose II, Lacto-N-hexaose, para-Lacto-N-hexaose, Disialyllacto-N-tetraose, Fucosyl-Lacto-N-hexaose, Difucosyl-Lacto-N-hexaose a, and Difucosyl-Lacto-N-hexaose b.

In certain embodiments, the concentrated human milk permeate composition includes some or all of 2′-fucosyllactose, 3-fucosyllactose, 3′-sialyllactose, 6′-sialyllactose, lacto-N-tetraose, lacto-N-neotetraose, lacto-N-fucopentaose I, lacto-N-fucopentaose II, lacto-N-fucopentaose III, sialyllacto-N-tetraose a, sialyllacto-N-tetraose b, sialyllacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-Lacto-N-hexaose a, difucosyl-Lacto-N-hexaose b, lactodifucotetraose, 6′galactosyllactose, 3′galactosyllactose, 3-Sialyl-3-fucosyllactose, sialylfucosyllacto-N-tetraose, sialyllacto-N-fucopentaose V, disialyllacto-n-fucopentaose II, disialyllacto-n-fucopentaose V, lacto-N-neo-difucohexaose II, 3-Fucosyl-sialylacto-N-tetraose c, para-Lacto-N-neohexose, lacto-N-octaose, lacto-N-neooctaose, lacto-N-neohexaose, lacto-N-fucopentaose V, iso-lacto-N-octaose, para-lacto-N-octaose, lacto-decaose, and sialyllacto-N-fucopentaose I.

In certain embodiments, the concentrated human milk permeate composition contains at least 10, 25, 50, 100, 125, or 150 HMOs which include all of 2′-fucosyllactose, lacto-N-tetraorose, 3-sialyllactose, 3-fucosyllactose, lacto-N-fucopentaose I, lacto-N-fucopentaose II, and 6′sialyllactose. In particular embodiments, the concentrated human milk permeate composition contains at least 25, 50, 100, 125, or 150 HMOs which include all of 2′-fucosyl-lacose, 3-fucosyllactose, 3′-sialyllactose, 6′-sialyllactose, lacto-N-tetraose, lacto-N-neotetraose, lacto-N-fucopentaose I, lacto-N-fucopentaose II, lacto-N-fucopentaose III, sialyllacto-N-tetraose b, sialyllacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-lacto-N-hexaose a, and difucosyl-Lacto-N-hexaose b. In particular embodiments, the concentrated human milk permeate composition contains at least 25, 50, 100, 125, or 150 HMOs which include all of 2′-fucosyllactose, 3-fucosyllactose, 3′-sialyllactose, 6′-sialyllactose, lacto-N-tetraose, lacto-N-neotetraose, lacto-N-fucopentaose I, lacto-N-fucopentaose II, lacto-N-fucopentaose III, sialyllacto-N-tetraose a, sialyllacto-N-tetraose b, sialyllacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-lacto-N-hexaose a, difucosyl-lacto-N-hexaose b, lactodifucotetraose (LD), 6′galactosyllactose, 3′galactosyllactose, 3-sialyl-3-fucosyllactose, sialylfucosyllacto-N-tetraose, sialyllacto-N-fucopentaose V, disialyllacto-n-fucopentaose II, disialyllacto-n-fucopentaose V, lacto-N-neo-difucohexaose II, 3-fucosyl-sialylacto-N-tetraose c, para-lacto-N-neohexose, lacto-N-octaose, lacto-N-neooctaose, lacto-N-neohexaose, lacto-N-fucopentaose V, iso-lacto-N-octaose, para-lacto-N-octaose, lacto-decaose, and sialyllacto-N-fucopentaose I.

In some embodiments, the concentrated human milk permeate composition has an increased amount, level, or concentration of one or more HMOs as compared to what is typically found human milk. In particular embodiments, the concentrated human milk permeate composition has an increased amount, level, or concentration of one or more HMOs as compared to what is typically found in untreated human milk permeate, e.g., permeate resulting from ultrafiltration of pooled human skim milk, such as described herein, or produced by a process described herein, e.g., in Section II-A-(i). In particular embodiments, the concentrated human milk permeate composition is or includes at least 25, 50, 75, 100, 125, 150, of the different HMOs found, present, or detected in pooled human milk (e.g., pooled from the milk of at least 10, 25, 50, or 100 individual donors) or in permeate (e.g., permeate resulting from ultra-filtering human milk skim) obtained from pooled human milk. In some embodiments, the concentrated human milk permeate composition is or includes at least 50%, 60%, 70%, 80%, 85%, 90%, 95%, 97%, 99%, or 99.9% of the different HMOs found, present, or detected in pooled human milk or in permeate) obtained from pooled human milk. In certain embodiments, the concentrated human milk permeate composition is or includes at least 50%, 60%, 70%, 80%, 85%, 90%, 95%, 97%, 99%, or 99.9% of the individual HMOs that may be found, present, or detected across samples of human milk. In some embodiments, the concentrated human milk permeate composition of HMOs is or includes the same or substantially the same HMOs found, present, or detected in pooled human milk or in permeate obtained from pooled human milk. In certain embodiments the concentrated human milk permeate composition is or includes a human milk permeate resulting from the ultrafiltration of human whole or skim milk pooled from milk collected from at least 10, 25, 50, or 100 individual human milk donors that is further concentrated, e.g., by nanofiltration or reverse osmosis, to increase the concentration of total HMO (e.g., by w/w). In some embodiments, the concentration of total HMO is increased to at least 0.5%, 1%, 2%, 3%, 4%, 5%, 6%, 7%, 8%, 9%, or 10%. In certain embodiments, the concentration of total HMO is increased to at least 5% (w/w). In certain embodiments, the concentration of total HMO is increased to between 8% and 12% (w/w).

In certain embodiments, the concentrated human milk permeate composition is free or essentially free of oligosaccharides that are not HMOs.

In some embodiments, the concentrated human milk permeate composition contains a plurality of HMOs that are or are derived from a concentrated ultra-filtered human milk permeate, e.g., any ultra-filtered human milk permeate described herein or produced by a method described herein such as in Section II-(A)-(i).

In some embodiments, the provided concentrated human milk permeate compositions have or include an HMO profile that is substantially similar both structurally and functionally to the profile of HMOs observed across the population of whole human milk. That is to say, in some aspects, since the prebiotics may be obtained from a source of human milk derived from a pool of donors, rather than an individual donor, the array of HMOs will be more diverse than in any one typical individual, and will represent or more closely represent the spectrum of HMOs that are found in human milk as opposed to the spectrum of HMOs that are found or typically found in the human milk produced by any particular individual.

In some embodiments, the concentrated human milk permeate composition is or includes a greater amount of different individual HMOs than the number of different individual HMOs found in human milk from an individual donor. In certain embodiments, the concentrated human milk permeate composition includes at least 1, 2, 3, 4, 5, 10, 15, 20, 25, 30, 35, 40, 45, or 50 more individual HMOs than the number of different individual HMOs found in human milk from an individual donor. In particular embodiments, a concentrated human milk permeate composition is or includes a greater amount of different individual HMOs than the mean or median number of different individual HMOs found in a plurality of human milk samples from individual donors. In certain embodiments, the concentrated human milk permeate composition includes at least 1, 2, 3, 4, 5, 10, 15, 20, 25, 30, 35, 40, 45, or 50 more individual HMOs than the number of different individual HMOs found in human milk from an individual donor.

In some aspects, one of the biggest variables in HMO diversity derives from the mother's Lewis blood group and specifically whether or not she has an active fucosyltrasferase 2 (FUT2) and/or fucosyltrasferase 3 (FUT3) gene. When there is an active FUT2 gene, an α1-2 linked fucose is produced, whereas fucose residues are α1-4 linked when the FUT3 gene is active. The result of this “secretor status” is, generally, that “secretors” (i.e. those with an active FUT2 gene) produce a much more diverse profile of HMOs dominated by α1-2 linked oligosaccharides, whereas “non-secretors” (i.e. those without an active FUT2 gene) may comprise a more varied array of, for example α1,-4 linked oligosaccharides (as compared to secretors), but comprise an overall decrease in diversity since they are unable to synthesize a major component of the secretor's HMO repertoire. In some embodiments, the concentrated human milk permeate composition includes human milk oligosaccharides that include α1-2 linked fucose and human milk oligosaccharides that include α1-4 linked fucose.

In some embodiments, the concentrated human milk permeate composition is or includes at least 5% total HMO (w/w). In particular embodiments, the concentrated human milk permeate composition is or includes least 5%, 6%, 7%, 8%, 9%, 10%, 11%, 12%, 15%, 20%, 25%, or 50% total HMO (w/w). In certain embodiments, the concentrated human milk permeate composition is or includes between 5% and 15%, 7.5% and 12.5%, 8% and 12%, 8.5% and 11%, or 8.4% and 10.6% total HMO (w/w). In certain embodiments, the concentrated human milk permeate composition is or includes between 8.5% and 11% total HMO (w/w). In some embodiments, the concentrated human milk permeate composition is or includes between 8.4% and 10.6% total HMO (w/w).

In some embodiments, the concentrated human milk permeate composition has a pH of between 4.0 and 5.5. In certain embodiments, the concentrated human milk permeate composition has less than 10%, 5%, 1%, or 0.1% lactose (w/w). In some embodiments, the concentrated human milk permeate composition has less than 10%, 5%, 1%, or 0.1% glucose (w/w). In particular embodiments, the concentrated human milk permeate composition has less than 10%, 5%, 1%, or 0.1% galactose (w/w). In certain embodiments, the concentrated human milk permeate composition has less than 10% galactose, less than 10% glucose, and less than 0.1% lactose.

In some embodiments, the concentrated human milk permeate composition is a liquid formulation. In some embodiments, the concentrated human milk permeate composition is in powdered form, e.g., a lyophilized or spray dried composition.

### I). Methods of Generating a Concentrated Human Milk Permeate Composition

In some embodiments, the concentrated human milk permeate composition is or includes human milk oligosaccharides (HMOs) obtained or purified from ultra-filtered permeate from donated human milk. In some embodiments, the permeate is concentrated to increase the concentration of HMOs. In certain embodiments, the donated human milk is pooled to provide a pool of human milk. In some embodiments, a pool of human milk comprises milk from two or more (e.g., ten or more) donors. In certain embodiments, the pooled human milk contains milk from at least 50, 75, 100, 150, or 200 individual donors. In certain embodiments, the pooled human milk contains human milk from at least 100 individual donors or between 100 and 300 individual donors. In some embodiments, the pooled human milk contains milk from at least ten, at least twenty-five, at least fifty, at least seventy-five, at least one hundred, or at least one hundred fifty individual human milk donors.

In some embodiments, the concentrated human milk permeate composition is or includes a concentrated, ultra-filtered permeate from pooled human milk. In some embodiments, the concentrated human milk permeate composition contains at least 10, 25, 30, 50, 75, 100, 125, 150 different individual HMO species (e.g., HMOs with different individual chemical formulas or chemical structures). In particular embodiments, the concentrated human milk permeate composition contains at least 50 HMOs which include all of 2′-fucosyllactose, 3-fucosyllactose, 3′-sialyllactose, 6′-sialyllactose, Lacto-N-tetraose, Lacto-N-neotetraose, Lacto-N-fucopentaose I, Lacto-N-fucopentaose II, Lacto-N-fucopentaose III, sialyllacto-N-tetraose b, sialyllacto-N-tetraose c, Lacto-N-difuco-hexaose I, Lacto-N-difuco-hexaose II, Lacto-N-hexaose, para-lacto-N-hexaose, disialyllacto-N-tetraose, fucosyl-Lacto-N-hexaose, difucosyl-lacto-N-hexaose a, and difucosyl-lacto-N-hexaose b.

In some aspects, the profile of HMOs contained by the concentrated human milk permeate composition is substantially similar both structurally and functionally to the profile or array of HMOs observed across the population of whole human milk. In certain embodiments, since the concentrated human milk permeate composition is derived from human milk obtained from a pool of donors rather than an individual donor, the profile or array of HMOs will be more diverse than in any one typical individual. In particular embodiments, the concentrated human milk permeate composition includes HMOs produced from secretor and non-secretor mothers. In some embodiments, the permeate contains or includes α1-2 linked HMOs and α1,4 linked HMOs.

In certain embodiments, the concentrated human milk permeate composition includes about or at least 0.1%, 0.5%, 1.0%, 1.5%, 2.0%, 2.5%, 3.0%, 4.0%, 5.0%, 7.5%, or 10% or more (w/w) of human milk oligosaccharides. In some embodiments, the concentrated human milk permeate composition is lyophilized or freeze-dried or otherwise powdered. In some embodiments, the permeate composition is an aqueous mixture.

In certain embodiments, the concentrated human milk permeate composition is produced from human milk permeate, e.g., concentrated ultra-filtered permeate from pooled human milk. In some embodiments, the concentrated human milk permeate composition contains or is formulated with human milk permeate, e.g., concentrated ultra-filtered permeate from pooled human milk. In some embodiments, the concentrated ultra-filtered permeate may be made according to any suitable method or technique known in the art. In some aspects, suitable methods and techniques include those described in U.S. Pat. No. 8,927,027 and PCT Pub. No. WO2018053535, hereby incorporated by reference in their entirety. Exemplary methods and techniques for producing the human milk compositions are briefly summarized herein.

In certain embodiments, the concentrated human milk permeate composition is or includes human milk permeate, e.g., permeate obtained by ultra-filtering human skim milk. In particular embodiments, the permeate is a concentrated ultra-filtered human milk permeate, e.g., ultra-filtered and concentrated as described herein, e.g., in Section-II-A-(i)-(a). In certain embodiments, the concentrated, ultra-filtered human milk permeate is derived or produced from an ultra-filtered human milk permeate that includes between or between about 84 g/L to 106 g/L HMO (w/v), at least 10 HMO including all of 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In certain embodiments, the concentrated human milk permeate composition is or includes no more than 1% lactose (by weight/weight or w/w), no more than 15% glucose (w/w), no more than 15% galactose (w/w), no more than 250 mg per 100 g calcium, no more than 250 mg per 100 g potassium, no more than 100 mg per 100 g magnesium, no more than 100 mg per 100 g sodium, and/or no more than 250 mg per 100 mg of phosphorus.

A). Processing Ultra-Filtered Permeate from Human Milk

In some embodiments, the donor milk is received frozen, and when desired, is thawed and pooled. In some embodiments, donor milk is then screened, e.g., to identify contaminants, by one or more of the methods discussed herein.

In some embodiments, the pooled milk is filtered, e.g., through about a 200-micron filter. In some embodiments, the pooled milk is heated, e.g., at about 63° C. or greater for about 30 minutes or more. In some embodiments, the milk is transferred to a separator, e.g., a centrifuge, to separate the cream from the skim. In some embodiments, the cream may go through separation once again to yield more skim. In some embodiments, a desired amount of cream is added to the skim prior to ultra-filtration. In certain embodiments, material that did not pass through the filter is collected as the retentate fraction, and material that passes through the filter is collected as the permeate fraction.

In some embodiments, the skim fraction undergoes ultra-filtration. In some embodiments, the ultrafiltration is performed with a filter between 1 kDa and 1000 kDa to obtain a protein rich retentate and the HMO-containing permeate. Details of this process can be found, for example, in U.S. Pat. Nos. 8,545,920; 7,914,822; 7,943,315; 8,278,046; 8,628,921; and 9,149,052, each of which is hereby incorporated by reference in its entirety. In some embodiments, the ultra-filtration is performed with a filter that is between 1 kDa and 100 kDa, 5 kDa and 50 kDa, or 10 kDa and 25 kDa. In certain embodiments, the filter is about or at least 5 kDa, 10 kDa, 20 kDa, 25 kDa, 50 kDa, or 100 kDa. In some embodiments, the skim fraction undergoes ultrafiltration with a filter that is about 10 kDa. In certain embodiments, the skim fraction undergoes ultrafiltration with a filter that is about 25 kDa. In particular embodiments, the skim fraction undergoes ultrafiltration with a filter that is about 50 kDa.

In some embodiments, the ultra-filtered permeate undergoes a process for reducing lactose. In certain embodiments, a process for producing a purified HMO composition with substantially reduced levels of lactose is provided. In certain embodiments, the substantial reduction includes or requires the biochemical and/or enzymatic removal of lactose from the lactose-rich human milk permeate fraction, without loss of yield or change in molecular profile of the HMO content of human milk permeate. And, in particular embodiments, without leaving residual inactivated foreign protein, if enzymatic digestion is used to reduce lactose. In certain embodiments, about or at least 50%, 60%, 70%, 80%, 90%, 95%, 99%, 99.9%, or 99.99% of the lactose present in the permeate after ultrafiltration is removed, e.g., enzymatically digested. In certain embodiments, the permeate is free or essentially free of lactose following the enzymatic digestion.

In certain embodiments, the process for reducing lactose from human milk permeate includes one or more of the steps of a) adjusting the pH of the permeate mixture; b) heating the pH adjusted mixture; c) adding lactase enzyme to the heated permeate mixture to create a permeate/lactase mixture and incubating for a period of time; d) removing the lactase from the mixture and filtering the mixture to remove lactase; and e) concentrating human milk oligosaccharides. In some embodiments, the order of when steps (a)-(c) are performed may be varied. Thus, in some aspects, the steps may be performed in the order of (a)-(b)-(c); (a)-(c)-(b); (c)-(b)-(a); (c)-(a)-(b); (b)-(a)-(c); or (b)-(c)-(a), such that, for example, the lactase enzyme may be added prior to heating the mixture, or, alternatively at any point during the heating process. Similarly, and also by way of example only, the mixture may be heated prior to adjustment of the pH. Furthermore, several steps may be grouped into a single step, for example “enzymatically digesting lactose” or “lactases digestion of lactose” involves steps (a)-(c) as described. These steps may be performed concurrently or consecutive in any order. Therefore, as used herein “lactose digestion” refers to the performance of at least these three steps, in any order, consecutively or concurrently.

In certain embodiments, the pH of the permeate is adjusted to a pH of about 3 to about 7.5. In some embodiments, the pH is adjusted to a pH of about 3.5 to about 7.0. In particular embodiments, the pH is adjusted to a pH of about 3.0 to about 6.0. In certain embodiments, the pH is adjusted to a pH of about 4 to about 6.5. In some embodiments, pH is adjusted to a pH of about 4.5 to about 6.0. In particular embodiments, the pH is adjusted to a pH of about 5.0 to about 5.5. In certain embodiments, the pH is adjusted to a pH of about 4.3 to about 4.7, preferably 4.5. The pH may be adjusted by adding acid or base. In some embodiments, pH is adjusted by adding acid, for example HCl. In particular embodiments, pH is adjusted by adding 1N HCl and mixing for a period of time e.g. about 15 minutes.

In some embodiments, the pH-adjusted permeate is heated to a temperature of about of about 25° C. to about 60° C. In certain embodiments, the permeate is heated to a temperature of about 30° C. to about 55° C. In some embodiments, the permeate is heated to a temperature of about 40° C. to about 50° C. In some embodiments, the permeate is heated to a temperature of about 40° C. to about 60° C., 45° C. to about 55° C., 47° C. to about 53° C., or 49° C. to about 51° C. In certain embodiments, the permeate is heated to a temperature of about 48° C. to about 50° C. In some embodiments, the permeate is heated to a temperature about 50° C. In some embodiments, the permeate is heated to a temperature less than or equal to about 40° C.

In particular embodiments, lactase enzyme is added to the pH-adjusted, heated permeate to create a permeate/lactase mixture. In certain embodiments, lactose within the permeate/lactase mixture is broken down into monosaccharides. In certain embodiments, lactase enzyme is added at about 0.1% w/w to about 0.5% w/w concentration. In certain embodiments, lactase enzyme is added at about 0.1% w/w, or 0.2% or 0.3% or 0.4% or 0.5% w/w. There are many commercially available lactase enzymes that may be used. As such, the lactase enzyme may be derived from any origin (e.g. fungal or bacterial in origin).

In some embodiments, the pH-adjusted, heated permeate is incubated with the lactase enzyme for about 5 to about 225 minutes. In certain embodiments, the incubation time is about 15 min to about 90 min. In some embodiments, the incubation time is about 30 minutes to about 90 minutes. In particular embodiments, the incubation time is about 60 minutes. Some aspects contemplate that incubation time is dependent upon myriad of factors including, but not limited to, the source of the enzyme used, the temperature and pH of the mixture and the concentration of enzyme used. Thus, in some embodiments, incubation time with the lactase enzyme may be adjusted to factor in such variables as a matter of routine. While the pH, temperature, and enzyme incubation conditions provided here are what work optimally for the process described herein, one of skill in the art would understand that modifications may be made to one or more of these variables to achieve similar results. For example, if less enzyme is used than the about 0.1% w/w to about 0.5% w/w described herein, the incubation time may need to be extended to achieve the same level of lactose digestion. Similar adjustments may be made to both the temperature and pH variables as well.

In certain embodiments, the permeate/lactase mixture is cooled to a temperature of about 20° C. to about 30° C. after the incubation. In a particular embodiment, the permeate/lactase mixture is cooled to a temperature of about 25° C.

In some embodiments, the permeate/lactase mixture is clarified to remove insoluble constituents. In certain embodiments, insoluble material may form throughout the change in pH and temperature. Thus, in some embodiments, it may be necessary or beneficial to clarify the mixture to remove these insoluble constituents, for example, through a depth filter. The filters may be 0.1 to 10 micron filters. In some embodiments, the filters are about 1 to about 5 micron filters. Alternatively, removal of insoluble constituents can be achieved through a centrifugation process or a combination of centrifugation and membrane filtration. The clarification step is not essential for the preparation of a diverse HMO composition, as described herein, rather, this optional step aids in obtaining a more purified permeate composition. Furthermore, the clarification step is important in the reusability of the filtration membranes and thus to the scalability of the process. Some aspects contemplate that, without adequate clarification, substantially more filter material is required, increasing the difficulty and expense to produce permeate compositions at clinical scale. However, one will understand that more or less stringent clarification may be formed at this stage in order to produce more or less purified permeate compositions, depending on formulation and application. For example, precipitated minerals may be less of a problem for a formulation destined for lyophilization.

In certain embodiments, the spent and excess lactase enzyme is removed from the clarified permeate/lactase mixture. There may, however, be some instances where the inactivated foreign protein will carry no biological risk and therefore the added steps of lactase removal or even inactivation may not be necessary. In some embodiments, the spent and excess lactase is inactivated, for example by high temperature, pressure, or both. In some embodiments, the inactivated lactase is not removed from the composition.

In certain embodiments, however, a further purification to remove foreign proteins is performed. In such embodiments lactase enzyme removal may be accomplished by ultrafiltration. In some embodiments, ultrafiltration is accomplished using an ultrafiltration membrane, for example using a membrane with molecular weight cut-off of ≤50,000 Dalton, e.g., a BIOMAX-50K, ≤25,000 Dalton, e.g., a BIOMAX-25K, or ≤10,000 Dalton e.g., a BIOMAX-10K. In some embodiments, the molecular weight cut-off is less than or equal to about 10 kDa. In certain embodiments, the molecular weight cut-off is less than or equal to about 25 kDa. In particular embodiments, the molecular weight cut-off is less than or equal to about 50 kDa.

In certain embodiments, an additional ultrafiltration is performed through a smaller membrane than the initial membrane) e.g., with molecular weight cut-off of ≤50,000, ≤25,000, or ≤10,000 Dalton. In some embodiments, the additional ultrafiltration is performed with a membrane with a molecular weight cut off of between 10 kDa and 50 kDa, 1 kDa and 10 kDa, 1 kDa and 5 kDa, or 2 kDa and 3 kDa. In certain embodiments, the additional ultrafiltration is performed with a membrane with a molecular weight cut off of between 2 kDa and 3 kDa. In certain embodiments, an additional ultrafiltration is not performed. In some embodiments, the additional filtration step is performed, such as to aid in the overall purity of the permeate product, such as by assisting in the removal of smaller potentially bioactive and/or immunogenic factors.

In some embodiments, the clarified mixture that has undergone at least one, and in some cases two or more rounds of ultrafiltration (or alternative lactase removal means) is further filtered to purify and concentrate human milk oligosaccharides and to reduce the mineral and monosaccharides content.

In some embodiments, filtration can be accomplished using a nanofiltration membrane. In some embodiments, the membrane has a molecular weight cut-off of ≤1,000 Dalton. In certain embodiments, the membrane has a molecular weight cut-off of between 1 kDa and 1,000 kDa. In certain embodiments, the membrane has a molecular weight cut-off of less than 600 Da. In certain embodiments, the membrane has a molecular weight cut-off between 400 Da and 500 Da. In some aspects, the additional nanofiltration removes monosaccharides, minerals, particularly calcium, and smaller molecules to produce the final purified HMO composition, e.g., the concentrated ultra-filtered human milk permeate.

In some embodiments, additional or alternative steps may be taken for the removal of minerals. Such an additional step may include, for example, centrifugation, membrane clarification (≤0.6 micron), or combination of centrifugation and membrane filtration of heated (≥40° C.) or refrigerated/frozen and thawing of concentrated ultra-filtered permeate, e.g., HMO concentrate. The collected supernatant or filtrate of these additional or alternative steps, in some embodiments, is concentrated further using a nanofiltration membrane. In some embodiments, the nanofiltration comprises filtration through a membrane with a molecular cut off of ≤600 Dalton. In some embodiments, these additional steps may be performed at any stage of the process, including but not limited to prior to or after pasteurization.

In some embodiments, the physical property of nanofiltration membranes can be modified, such as chemical modification, to selectively concentrate sialylated HMOs, for example, allowing greater efficiency of neutral HMOs removal from HMO concentrate, in instances where concentrated sialylated HMOs are preferred.

In some embodiments, the permeate is treated to reduce bioburden, such as by any means known in the art. In some embodiments, the purified HMO composition is pasteurized. In some aspects, pasteurization is accomplished at ≥63° C. for a minimum of 30 minutes. Following pasteurization, the composition is cooled to about 25° C. to about 30° C. and clarified through a 0.2 micron filter to remove any residual precipitated material.

In certain embodiments, the permeate may be further processed, e.g., concentrated or diluted. In some embodiments, the permeate may be concentrated by a suitable process such as nanofiltration, reverse osmosis, or dried, e.g., lyophilized. In some embodiments, the purified HMO compositions made by the methods herein may be lyophilized or freeze-dried or otherwise powdered.

### b.) Obtaining Pooled Human Milk

In some embodiments, permeate is obtained from human milk pooled from multiple qualified human milk donors. The process for obtaining, testing, and qualifying the pooled human milk is described briefly herein.

In some embodiments, the human milk is provided by donors, and the donors are pre-screened and approved before any milk is processed. In some aspects, various techniques are used to identify and qualify suitable donors. In some embodiments, a potential donor must obtain a release from her physician and her child's pediatrician as part of the approval process. This helps to insure, inter alia, that the donor is not chronically ill and that her child will not suffer as a result of the donation(s). Methods and systems for qualifying and monitoring milk collection and distribution are described, e.g., in U.S. Pat. Nos. 8,545,920; 7,943,315; 9,149,052; 7,914,822 and 8,278,046, which are incorporated herein by reference in its entirety. Donors may or may not be compensated for their donation.

In certain embodiments, the donor screening includes a comprehensive lifestyle and medical history questionnaire that includes an evaluation of prescription and non-prescription medications, testing for drugs of abuse, and testing for certain pathogens. In some embodiments, a biological sample, e.g., a blood sample and/or a milk sample, may be screened for the presence of an infectious agent such as a bacteria or virus by any suitable routine technique, e.g., qPCR or ELISA. Such infectious agents may include, but are not limited to, human immunodeficiency virus Type 1 (HIV-1), HIV-2, human T-lymphotropic virus Type 1 (HTLV-I), HTLV-II, hepatitis B virus (HBV), hepatitis C virus (HCV), and syphilis.

In some embodiments, donors may continuously provide samples over a period of time, e.g., about or at least one month, three months, six months, a year, or over a year. In some embodiments, during the period of time, the donor may be requalified. In some aspects, a donor who does not requalify or fails qualification is deferred until such time as they do, or permanently deferred if warranted by the results of requalification screening. In the event of the latter situation, all remaining milk provided by that donor is removed from inventory and destroyed or used for research purposes only.

In some embodiments, once the donor has been approved, donor identity matching may be performed on donated human milk such as to ensure that the donated milk was expressed from the qualified donor and not another, e.g., when the milk was expressed by a donor away from the milk banking facility. In particular embodiments, the donor's milk may be sampled for genetic markers, e.g., DNA markers, to guarantee that the milk is truly from the approved donor. Such subject identification techniques are known in the art (see, e.g., U.S. Pat. No. 7,943,315, which is incorporated herein by reference in its entirety). In some embodiments, milk may be stored (e.g., at −20° C. or colder) and quarantined until the test results are received.

The milk is also tested for pathogens. In some embodiments, the milk is genetically screened, e.g., by polymerase chain reaction (PCR), to identify, e.g., viruses, such as HIV-1, HBV and HCV. In some embodiments, a microorganism panel that screens for various bacterial species, fungus and mold via culture may also be used to detect contaminants. In some embodiments, a microorganism panel may test for aerobic count, Bacillus cereus, Escherichia coli, Salmonella, Pseudomonas, coliforms, Staphylococcus aureus, yeast, and mold. In some embodiments, pathogen screening may be performed both before and after pasteurization.

In addition to screening for pathogens, the donor milk may also be tested for drugs of abuse (e.g., including but not limited to cocaine, opiates, synthetic opioids (e.g. oxycodone/oxymorphone) methamphetamines, benzodiazepine, amphetamines, and THC) and/or adulterants such as non-human proteins. In certain embodiments, an ELISA may be used to test the milk for a non-human protein, such as bovine proteins, to ensure, e.g., that cow milk or cow milk infant formula has not been added to the human milk, for example to increase donation volume when donors are compensated for donations.

In certain embodiments, adulterants may include any non-human milk fluid or filler that is added to a human milk donation, thereby causing the donation to no longer be unadulterated, pure human milk. Particular adulterants to be screened for include non-human milk and infant formula. In particular embodiments, the adulterants that are screened for include cow milk, cow milk formula, goat milk, soy milk, and soy formula. In some embodiments, methods that are known and routine by those of skill in the art may be adapted to detect non-human milk proteins, e.g., cow milk and soy proteins, in a human milk sample. In particular, immunoassays that utilize antibodies specific for a protein found in an adulterant that is not found in human milk can be used to detect the presence of the protein in a human milk sample, e.g., an enzyme-linked immunosorbent assay (ELISA), a western blot, or immunoblot.

### B.) Synthetic Oligosaccharides

In particular embodiments, the one or more synthetic oligosaccharides are administered to a subject, e.g., to promote or maintain engraftment of the at least one probiotic strain within the subject's gut or intestinal microbiome. In some embodiments, the one or more synthetic oligosaccharides are non-digestible carbohydrates that promote the growth or expansion of the at least one probiotic strain, e.g., in vivo such as in the human gut or intestinal microbiome. In certain embodiments, the one or more synthetic oligosaccharides, e.g., non-digestible carbohydrates such as human milk oligosaccharides, promotes, e.g., selectively or exclusively, the colonization, expansion, extension, or increased presence of the at least one probiotic strain within the microbiome. In particular embodiments, the one or more synthetic oligosaccharides promotes the growth or expansion of a Bifidobacterium probiotic strain such as B. longum subsp. infantis, e.g., in vivo such as in the human gut or intestinal microbiome.

In some embodiments, the one or more synthetic oligosaccharides is or includes synthetic non-digestible carbohydrates. In various embodiments, the one or more synthetic oligosaccharides is or includes one or more synthesized oligosaccharides that are identical to those found in or naturally occurring in a mammalian milk. In certain embodiments, the one or more synthetic oligosaccharides is or includes one or more synthetic human milk oligosaccharides.

In certain embodiments, the synthetic oligosaccharides may include one or more of a fructo-oligosaccharide (FOS), galactooligosaccharide (GOS), transgalactooligosaccharide (TOS), gluco-oligosaccharide, xylo-oligosaccharide (XOS), chitosan oligosaccharide (COS), soy oligosaccharide (SOS), isomalto-oligosaccharide (IMOS), or derivatives thereof. In certain embodiments, such derivatives include those with modifications that may increase the likelihood or probability of consumption, metabolism, and/or internalization (such as by transport or import) of the oligosaccharide by the probiotic strain, e.g., B. longum subsp. infantis. Such modifications may include but are not limited to fucosylation or sialylation. In some embodiments, the synthetic oligosaccharides may include one or more of a FOS, GOS, TOS, gluco-oligosaccharide, XOS, COS, SOS, IMOS, or derivatives or any or all of the foregoing, that are capable of being metabolized, consumed, and/or internalized by one or more strains, species, or subspecies of Bifidobacterium, e.g., B. longum subsp. infantis. In certain embodiments, the synthetic oligosaccharides include one or more oligosaccharides that are obtained or derived from a resistant starch, pectin, psyllium, arabinogalactan, glucomannan, galactomannan, xylan, lactosucrose, lactulose, lactitol and various other types of gums such as tara gum, acacia, carob, oat, bamboo, citrus fibers, such as by treatment with enzymes that hydrolyze fiber or polysaccharides. In some embodiments, the one or more synthetic oligosaccharides that are obtained by these means are capable of being consumed, metabolized, and/or internalized by at least one strain of Bifidobacterium such as B. longum subsp. infantis.

In certain embodiments, the one or more synthetic oligosaccharides are identical, e.g., by chemical structure or formula, to oligosaccharides found in a mammalian milk. In some embodiments, the synthetic oligosaccharides may be internalized and metabolized by certain strains of Bifidobacterium, e.g., B. longum subsp. infantis. In certain embodiments, the one or more synthetic oligosaccharides are identical to one or more mammalian milk oligosaccharides. In certain embodiments, the one or more synthetic oligosaccharides are synthetic mammalian milk oligosaccharides. In particular embodiments, the one or more synthetic oligosaccharides are identical to one or more oligosaccharides that are found in a milk that includes but is not limited to milk from dog, cat, camel, goat, cow, yak, buffalo, horse, donkey, zebu, sheep, reindeer, giraffe, elephant, non-human primate, or human.

In certain embodiments, the one or more synthetic oligosaccharides are synthetic human milk oligosaccharides. In certain embodiments, the synthetic human milk oligosaccharides are oligosaccharides that are synthesized, produced, derived, obtained, or manufactured from a non-human milk source. In some aspects, synthetic human milk oligosaccharides, as well as methods for synthesizing oligosaccharides and human milk oligosaccharides, are known, and include but are not limited to those described in PCT Publication Nos.: WO2017101958, WO2015197082, WO2015032413, WO2014167538, WO2014167537, WO2014135167, WO2013190531, WO2013190530, WO2013139344, WO2013182206, WO2013044928, WO2019043029, WO2019008133, WO2018077892, WO2017042382, WO2015150328, WO2015106943, WO2015049331, WO2015036138, and WO2012097950, each of which is incorporated by reference herein in its entirety.

In some embodiments, the one or more synthetic human milk oligosaccharides is or includes one or more of some or all of 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In certain embodiments, the one or more synthetic human milk oligosaccharides is or includes one or more of 2-fucosyllactose, lacto-N-tetraorose, 3-sialyllactose, 3-fucosyllactose, lacto-N-fucopentaose I, lacto-N-fucopentaose II, and 6′sialyllactose. In certain embodiments, the one or more synthetic human milk oligosaccharides includes one or more of 2′-fucosyllactose, 3-fucosyllactose, 3′-sialyllactose, 6′-sialyllactose, lacto-N-tetraose, lacto-N-neotetraose, lacto-N-fucopentaose I, lacto-N-fucopentaose II, lacto-N-fucopentaose III, sialyllacto-N-tetraose a, sialyllacto-N-tetraose b, sialyllacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-Lacto-N-hexaose a, difucosyl-lacto-N-hexaose b, lactodifucotetraose (LD), 6′galactosyllactose, 3′galactosyllactose, 3-sialyl-3-fucosyllactose, sialylfucosyllacto-N-tetraose, sialyllacto-N-fucopentaose V, disialyllacto-n-fucopentaose II, disialyllacto-n-fucopentaose V, lacto-N-neo-difucohexaose II, 3-fucosyl-sialylacto-N-tetraose c, para-lacto-N-neohexose, lacto-N-octaose, lacto-N-neooctaose, lacto-N-neohexaose, lacto-N-fucopentaose V, iso-Lacto-N-octaose, para-lacto-N-octaose, lacto-decaose, and sialyllacto-N-fucopentaose I.

In certain embodiments, the one or more synthetic human milk oligosaccharides is or includes one or more of 2′-fucosyllactose, 3-fucosyllactose, lacto-N-tetraose, or lacto-N-neotetraose. In particular embodiments, the one or more synthetic human milk oligosaccharides is or includes two or more of 2′-fucosyllactose, 3-fucosyllactose, lacto-N-tetraose, or lacto-N-neotetraose. In some embodiments, the one or more synthetic human milk oligosaccharides is or includes 2′-fucosyllactose. In certain embodiments, the one or more synthetic human milk oligosaccharides is or includes 3-fucosyllactose. In particular embodiments, the one or more synthetic human milk oligosaccharides is or includes lacto-N-tetraose. In some embodiments, the one or more synthetic human milk oligosaccharides is or includes lacto-N-neotetraose. In certain embodiments, the one or more synthetic human milk oligosaccharides is or includes (i) 2′-fucosyllactose and 3-fucosyllactose, (ii) 2′-fucosyllactose and lacto-N-tetraose, (iii) 2′-fucosyllactose and lacto-N-neotetraose, (iv) 3-fucosyllactose and lacto-N-tetraose, (v) 3-fucosyllactose and lacto-N-neotetraose, or (vi) lacto-N-tetraose and lacto-N-neotetraose. In particular embodiments, the one or more synthetic human milk oligosaccharides is or includes (i) 3-fucosyllactose, lacto-N-tetraose, and lacto-N-neotetraose, (ii) 2′-fucosyllactose, Lacto-N-tetraose, and Lacto-N-neotetraose, (iii) 2′-fucosyllactose, 3-fucosyllactose, and lacto-N-neotetraose, or (iv) 2′-fucosyllactose, 3-fucosyllactose, and lacto-N-tetraose. In some embodiments, the one or more synthetic human milk oligosaccharides are or include 2′-fucosyllactose, 3-fucosyllactose, lacto-N-tetraose, and lacto-N-neotetraose.

In certain embodiments, the one or more synthetic human milk oligosaccharides is or includes 2′-fucosyllactose. In particular embodiments, the one or more synthetic human milk oligosaccharides is or includes disialyllacto-N-tetraose.

### C.) Probiotics

In particular embodiments, provided herein are compositions that are or include at least one probiotic strain of bacteria, e.g., a strain of Bifidobacterium such as B. longum subsp. infantis. In some embodiments, the at least one probiotic strain, e.g., B. longum subsp. infantis, is contained or included in the same composition as the prebiotics. In certain embodiments, the at least one probiotic strain, e.g., B. longum subsp. infantis, is contained or included in a separate composition from the prebiotics.

In particular embodiments, the at least one probiotic strain is capable of consuming or metabolizing oligosaccharides such as HMOs. In some embodiments, the at least one probiotic strain is capable of utilizing HMOs as a carbon source. In particular embodiments, HMOs are preferentially consumed or metabolized by the at least one probiotic strain, e.g., as compared to other microbes or bacteria present in the gut or microbiome. In certain embodiments, the at least one probiotic strain is capable of consuming or metabolizing one or more prebiotics, including of those described herein, e.g., in Section II-A or II-B. In certain embodiments, the at least one probiotic strain is capable of consuming or metabolizing all or essentially all of the oligosaccharides of the concentrated human milk permeate composition.

In certain embodiments, the at least one probiotic strain is capable of consuming or metabolizing HMOs. In some embodiments, the at least one probiotic strain is capable of internalizing HMOs prior to consuming or metabolizing the HMOs. Particular embodiments contemplate that probiotic strains that consume or metabolize HMOs are known and may be identified by routine techniques such as those described in Gotoh et al. Sci Rep. 2018 Sep. 18; 8(1):13958, incorporated by reference herein in its entirety.

In some embodiments, the at least one probiotic strain contains one or more enzymes capable of hydrolyzing the prebiotics. In certain embodiments, the at least one probiotic strain contains one or more enzymes capable of hydrolyzing the human milk oligosaccharides. In particular embodiments, the one or more enzymes hydrolyze external oligosaccharides, e.g., oligosaccharides such as HMOs that are outside of the probiotic cell. In some embodiments, the one or more enzymes hydrolyze oligosaccharides such as human milk oligosaccharides internally or within the probiotic cell. In certain embodiments, the one or more enzymes hydrolyze internalized human milk oligosaccharides.

In particular embodiments, the at least one probiotic strain contains one or more enzymes capable of hydrolyzing one or more HMOs. In particular embodiments, the one or more enzymes hydrolyze external HMOs. In some embodiments, the one or more enzymes hydrolyze HMOs that are outside of the probiotic cell. In some embodiments, the one or more enzymes hydrolyze HMOs internally. In particular embodiments, the one or more enzymes hydrolyze HMOs within the probiotic cell. In certain embodiments, the one or more enzymes hydrolyze internalized HMOs.

In some embodiments, the at least one probiotic strain is capable of internalizing human milk oligosaccharides. In certain embodiments, the at least one probiotic strain internalizes human milk oligosaccharides prior to hydrolyzing the human milk oligosaccharides. In various embodiments, the at least one probiotic selectively or exclusively utilizes human milk oligosaccharides as a carbon source. Thus, in certain embodiments, if the at least one probiotic is administered to the subject and/or has engrafted, e.g., within the subject's microbiome (such as the intestinal microbiome), the at least one probiotic is present, expands, or increases in amount within the subject's microbiome when human milk oligosaccharides are administered to and/or ingested by the subject, and, in certain embodiments, the at least one probiotic is no longer present and/or decreases in amount within the subject's microbiome when the human milk oligosaccharides are no longer ingested or administered.

In some embodiments, the at least one probiotic strain is capable of internalizing oligosaccharides, such as to consume or metabolize the oligosaccharides. In certain embodiments, the at least one probiotic strain is capable of internalizing one or more oligosaccharides, including those of any of the oligosaccharides described herein, e.g., in Section II-A or II-B. In certain embodiments, the at least one probiotic strain is capable of internalizing HMOs.

In certain embodiments, the at least one probiotic strain is one or more of a Bifidobacterium, Lactobacillus, Clostridium, Eubacterium, or Streptococcus strain, e.g., capable of consuming or metabolizing HMOs. In certain embodiments, the at least one probiotic strain is or includes at least one strain of Bifidobacterium such as, but not limited to, B. adolescentis, B. animalis (e.g., B. animalis subsp. animalis or B. animalis subsp. lactis), B. bifidum, B. breve, B. catenulatum, B. longum (e.g., B. longum subsp. infantis or B. longum subsp. longum), B. pseudocatanulatum, B. pseudolongum; and/or at least one strain of Lactobacillus, such as L. acidophilus, L. antri, L. brevis, L. casei, L. coleohominis, L. crispatus, L. curvatus, L. delbrueckii, L. fermentum, L. gasseri, L. johnsonii, L. mucosae, L. pentosus, L. plantarum, L. reuteri, L rhamnosus, L. sakei, L. salivarius, L. paracasei, L. kisonensis., L. paralimentarius, L. perolens, L. apis, L. ghanensis, L. dextrinicus, L. harbinensis; and/or at least one strain of Bacteroides such as Bacteroides vulgatus or non-toxigenic Bacteroides fragilis; and/or at least one strain of Clostridium such as C. difficile or C. perfringens; and/or at least one strain of Eubacterium such as E. rectale; and/or at least one species of Streptococcus such as S. thermophilus; and/or at least one strain of Faecalibacterium such as Faecalibacterium prausnitzii, and/or at least one strain of Pediococcus, such as P. parvulus, P. lolii, P. acidilactici, P. argentinicus, P. claussenii, P. pentosaceus, or P. stilesii; and/or at least one strain of Lactococcus lactis. In some embodiments, the one or more probiotic may contain more than one strain, such as two or more of any of the species listed herein. As used herein, the terms “B. longum subsp. infantis” and “B. infantis” are be used interchangeably unless otherwise indicated. The terms “B. longum subsp. longum” and “B. longum” are also used interchangeably herein, unless indicated otherwise.

In particular embodiments, the at least one probiotic strain is one or more of a strain of B. longum subsp. infantis, B. bifidum, Bacteroides fragilis, Bacteroides vulgatus, Faecalibacterium prausnitzii, Eubacterium rectale, Lactobacillus acidophilus, Lactobacillus delbrueckii, Lactococcus lactis, or Streptococcus thermophilus, e.g., that is capable of consuming or metabolizing HMOs. In some embodiments, the at least one probiotic strain is one or more strains of B. longum subsp. infantis, B. bifidum, Bacteroides fragilis, or Bacteroides vulgatus, e.g., that is capable of consuming or metabolizing HMOs.

In particular embodiments, the species or subspecies of a given probiotic strain may be identified by routine techniques. For example, in some embodiments, the species or subspecies is identified by assessing the sequence similarity of one or more genes to corresponding sequences of known members of bacterial species or subspecies. In certain embodiments, a probiotic strain falls within a species or subspecies if all or a portion of its 16S gene has at least 97% sequence identity to all or a portion of a known 16S sequence of a known strain falling within the species. In particular embodiments, a probiotic strain falls within a species or subspecies if all or a portion of its 16S gene has at least 97% sequence identity to all or a portion of a known 16S sequence of a known strain falling within the species. Exemplary full or partial 16S sequences are summarized in Table 1.

TABLE 1
Exemplary 16S sequences

| Bacterial species or subspecies | Exemplary 16S Sequence |
|---|---|
| B. longum subsp. infantis | SEQ ID NOS: 1-7 |
| B. adolescentis | SEQ ID NO: 8 |
| B. animalis subsp. animalis | SEQ ID NO: 9 |
| B. animalis subsp. lactis | SEQ ID NO: 10 |
| B. bifidum | SEQ ID NO: 11 |
| B. breve | SEQ ID NO: 12 |
| B. catenulatum | SEQ ID NO: 13 |
| B. longum subsp. longum | SEQ ID NO: 14 |
| B. pseudocatanulatum | SEQ ID NO: 15 |
| B. pseudolongum | SEQ ID NO: 16 |
| L. acidophilus | SEQ ID NO: 17 |
| L. antri | SEQ ID NO: 18 |
| L. brevis | SEQ ID NO: 19 |
| L. casei | SEQ ID NO: 20 |
| L. coleohominis | SEQ ID NO: 21 |
| L. crispatus | SEQ ID NO: 22 |
| L. curvatus | SEQ ID NO: 23 |
| L. delbrueckii | SEQ ID NO: 24 |
| L. fermentum | SEQ ID NO: 25 |
| L. gasseri | SEQ ID NO: 26 |
| L. johnsonii | SEQ ID NO: 27 |
| L. harbinensis | SEQ ID NO: 28 |
| L. mucosae | SEQ ID NO: 29 |
| L. pentosus | SEQ ID NO: 30 |
| L. plantarum | SEQ ID NO: 31 |
| L. reuteri | SEQ ID NO: 32 |
| L rhamnosus | SEQ ID NO: 33 |
| L. sakei | SEQ ID NO: 34 |
| L. salivarius | SEQ ID NO: 35 |
| L. paracasei | SEQ ID NO: 36 |
| L. kisonensis | SEQ ID NO: 37 |
| L. paralimentarius | SEQ ID NO: 38 |
| L. perolens | SEQ ID NO: 39 |
| L. apis | SEQ ID NO: 40 |
| L. ghanensis | SEQ ID NO: 41 |
| L. dextrinicus | SEQ ID NO: 42 |
| Lactococcus lactis | SEQ ID NO: 43 |
| Bacteroides vulgatus | SEQ ID NO: 44 |
| Bacteroides fragilis | SEQ ID NO: 45 |
| Faecalibacterium prausnitzii | SEQ ID NO: 46 |
| E. rectale | SEQ ID NO: 47 |
| S. thermophilus | SEQ ID NO: 48 |
| P. parvulus | SEQ ID NO: 49 |
| P. lolii or P. acidilactci | SEQ ID NO: 50 or 51 |
| P. argentinicus | SEQ ID NO: 52 |
| P. claussenii | SEQ ID NO: 53 |
| P. pentosaceus | SEQ ID NO: 54 |
| P. stilesii | SEQ ID NO: 55 |

In certain embodiments, the at least one probiotic strain has or includes a nucleic acid sequence with at least 97%, at least 98%, at least 99%, or at least 99.5% identity to a nucleic acid sequence set forth in any of SEQ ID NOS: 1-55. In particular embodiments, the at least one probiotic strain has or includes a nucleic acid sequence with at least 97% at least 98%, at least 99% or at least 99.5% identity to a nucleic acid sequence set forth in any of SEQ TD NOS: 1-16 or 43-46. In certain embodiments, the at least one probiotic strain has or includes a nucleic acid sequence with at least 97% at least 980%, at least 99% or at least 99.5% identity to a nucleic acid sequence set forth in any of SEQ TD NOS: 1-7, 11, 12, 17, 24, or 43-47. In some embodiments, the at least one probiotic strain has or includes a nucleic acid sequence with at least 97% at least 980%, at least 99% or at least 99.5% identity to a nucleic acid sequence set forth in any of SEQ TD NOS: 1-7, 11, 44, or 45. In certain embodiments, the at least one probiotic strain has or includes a nucleic acid sequence with at least 97%, at least 98%, at least 99%, or at least 99.5% identity to a nucleic acid sequence set forth in any of SEQ TD NOS: 1-16. In particular embodiments, the at least one probiotic strain has or includes a nucleic acid sequence with at least 97%, at least 98%, at least 99%, or at least 99.5% identity to a nucleic acid sequence set forth in any of SEQ ID NOS: 1-7.

In particular embodiments, the at least one probiotic strain is or includes a strain of B. longum subsp. infantis. In particular embodiments, the strain of B. longum subsp. infantis has or includes a nucleic acid sequence with at least 97%, at least 98%, at least 99%, or at least 99.5% identity to a nucleic acid sequence set forth in any of SEQ ID NOS: 1-7. In particular embodiments, the strain of B. longum subsp. infantis has or includes a nucleic acid sequence of at least 200, 300, 400, 500, 600, 700, 800, 900, 1,000, 1,200, or 1,500 nucleotides in length with at least 60%, 70%, 80%, 90%, 95%, 99%, or 99.9% sequence identity to a nucleic acid sequence set forth in SEQ ID NOS: 59-78. In some embodiments, the strain of B. longum subsp. infantis has or includes a nucleic acid sequence having at least 60%, 70%, 80%, 90%, 95%, 99%, or 99.9% sequence identity to a nucleic acid sequence set forth in SEQ ID NOS: 59-78. In certain embodiments, the strain of B. longum subsp. infantis has or includes a nucleic acid sequence having at least 70%, 80%, or 90%, sequence identity to a nucleic acid sequence set forth in SEQ ID NOS: 59-69. In some embodiments, the strain of B. longum subsp. infantis has or includes a nucleic acid sequence having at least 80%, 85%, or 90% sequence identity to a nucleic acid sequence set forth in SEQ ID NOS: 70-74. In particular embodiments, the strain of B. longum subsp. infantis has or includes a nucleic acid sequence having at least 90%, 95%, or 99% sequence identity to a nucleic acid sequence set forth in SEQ ID NOS: 75-78. In some embodiments, the strain of B. longum subsp. infantis has or includes nucleic acid sequences having at least 90%, 95%, or 99% sequence identity to one or more of the nucleic acid sequences set forth in SEQ ID NOS: 59-78. In some embodiments, the strain of B. longum subsp. infantis has or includes the nucleic acid sequences set forth in one or more of SEQ ID NOS: 59-78. In particular embodiments, the strain of B. longum subsp. infantis has or includes nucleic acid sequences having at least 90%, 95%, or 99% sequence identity to all of the nucleic acid sequences set forth in SEQ ID NOS: 59-78. In various embodiments, the strain of B. longum subsp. infantis has or includes the nucleic acid sequences set forth in SEQ ID NOS: 59-78.

In some embodiments, the at least one probiotic strain is or includes a strain of Bifidobacterium or a Bacteroides capable of consuming, metabolizing, and/or internalizing HMOs. In some aspects, HMO cannot be metabolized by the host, e.g., mammals such as humans, or most bacteria, including many species of pathogenic bacteria and most bacteria commonly found in the microbiome of adult humans. In particular aspects, some strains, species, or subspecies of Bifidobacterium, such as B. longum subsp. infantis, or Bacteroides have enzymatic activity able to degrade specific alpha and beta bonds of HMOs. Five monosaccharides can be found in different HMO structures, glucose, galactose, N-acetyl glucosamine, fucose, and sialic acid (also referred to herein as N-acetyl neuraminic acid). Some strains, species, or subspecies of Bifidobacterium are able to fully degrade HMO intracellularly. Such Bifidobacterium possess genes encoding specific transporters (e.g., ABC transporters such as those described in Sela et al. PNAS (2008) 105 (48) 18964-18969; Schell, et al. PNAS. (2002) 99(22):14422-14427 and LoCascio et al. Appl Environ Microbiol. (2010) 76(22):7373-81), incorporated by reference herein, that selectively transport or import HMO and enzymes necessary for HMO degradation (alpha-fucosidase, alpha-sialidase, beta-galactosidase, and beta-N-hexosaminidase). Other Bifidobacterium strains, such as for example B. bifidum degrades HMO externally or extracellularly, such as for example by lacto-N-biosidase, which cleaves lacto-N-biose I (LNB) from HMO. The LNB is then internalized by a transporter and degraded by LNB-phosphorylase. In some embodiments, the at least one probiotic strain is at least one strain of bacterium having one or more genes encoding all or a portion of a transporter, e.g., an ABC transporter, capable of internalizing an oligosaccharide such as an HMO. In particular embodiments, the at least one probiotic strain is a bacterium having one or more genes encoding one or more enzymes, e.g., alpha-fucosidase, alpha-sialidase, beta-galactosidase, and beta-N-hexosaminidase, capable degrading an oligosaccharide such as an HMO. In certain embodiments, the at least one probiotic strain is at least one strain of Bifidobacterium or Bacteroides having one or more genes encoding all or a portion of a transporter, e.g., an ABC transporter, capable of internalizing an oligosaccharide, e.g., an HMO.

In some embodiments, the at least one probiotic strain is B. longum subsp. infantis. Particular embodiments contemplate that B. longum subsp. infantis is known and readily identifiable by those of skill in the art using routine techniques. In some embodiments, B. longum subsp. infantis, including its genome and biology, are known and for example have been described, including in Sela et al. PNAS (2008) 105 (48) 18964-18969; Underwood et al., Pediatr Res. (2015) 77(0): 229-235, incorporated by reference herein. In certain embodiments, Bifidobacterium, e.g., B. longum subsp. infantis, may be isolated using known selective microbiological media, e.g., De Man, Rogosa and Sharpe agar (MRS), optionally in combination with mupirocin, or those described in O'Sullivan et al., J Appl Microbiol. 2011 August; 111(2):467-73, incorporated by reference herein. In some embodiments, suitable sources for isolating Bifidobacterium, e.g., B. infantis, are known, and include stool samples obtained from breast fed infants. In certain embodiments, bacterial colonies may be identified or characterized by routine biochemical techniques, such as PCR. In some embodiments, B. longum subsp. infantis is identified by tagman qPCR, such as described in Lawley et al., PeerJ. 2017 May 25; 5: e3375. e.g., as performed with forward primer sequence ATACAGCAGAACCTTGGCCT (SEQ ID NO: 56), reverse primer sequence GCGATCACATGGACGAGAAC (SEQ ID NO: 57) and probe sequence [FAM dye]-TTTCACGGA-[ZEN quencher]-TCACCGGACCATACG-[3IABkFQ quencher](SEQ ID NO: 58). In some aspects, a strain may be confirmed as B. longum subsp. infantis by observing growth when HMOs are provided as the sole carbon source, such as with an assay described in Gotoh et al. Sci Rep. 2018 Sep. 18; 8(1):13958, incorporated by reference herein.

### D.) Exemplary Compositions, Kits, and Articles of Manufacture

In some embodiments, provided herein are compositions, kits, or articles of manufacture that are or include a combination of prebiotics, e.g., a concentrated human milk permeate composition and/or synthetic oligosaccharides, and at least one probiotic, e.g., a strain of Bifidobacteria such as B. longum subsp. infantis. In certain aspects, the prebiotics and/or the probiotic strain may be formulated as a pharmaceutical composition or a nutritional composition. In particular embodiments, the at least one probiotic strain may be formulated as a pharmaceutical composition or a nutritional composition. In certain embodiments, the prebiotics and the at least one probiotic strain are contained within separate compositions. In some embodiments, provided herein are kits or articles of manufacture that are or include separate prebiotic and probiotic compositions.

In some embodiments, provided herein are kits or articles of manufacture that are or include a composition of prebiotics that contain one or more human milk oligosaccharides and a composition that is or includes at least one strain of probiotic bacteria. In certain embodiments, the probiotic strain is capable of consuming (e.g., hydrolyzing) the prebiotics of the concentrated human milk permeate composition and/or the synthetic oligosaccharides. In particular embodiments, the probiotic strain is capable of internalizing and consuming (e.g., hydrolyzing) the prebiotics. In various embodiments, the probiotic strain is capable of internalizing and consuming (e.g., hydrolyzing) human milk oligosaccharides. In particular embodiments, the probiotic strain is capable of consuming, internalizing, and/or hydrolyzing a prebiotic in vivo such as within the human gut.

In particular embodiments, formulations for use in accordance with the invention are or include prebiotics and a probiotic strain for administration to the subject. The prebiotics and the probiotic strain may be administered simultaneously, separately and/or sequentially in relation to each other. In certain embodiments, the prebiotics are or include human milk oligosaccharides and the at least one probiotic strain is or includes any of the probiotic strains listed in Table 1.

In some embodiments, the kits or articles of manufacture are or include (i) a concentrated human milk permeate composition comprising human milk oligosaccharides, (ii) one or more synthetic oligosaccharides, and (iii) at least one probiotic strain of bacterium capable of consuming human milk oligosaccharides. In various embodiments, the at least one probiotic is or includes a strain of Bifidobacteria. In particular embodiments, the strain of Bifidobacteria is or includes B. longum subsp. infantis. In some embodiments, the one concentrated human milk permeate composition is or includes at least at least 5, 10, 25, 50, or 100 human milk oligosaccharides. In particular embodiments, the concentrated human milk permeate composition is produced by a method described herein, e.g., in Section-II-A-(i). In certain embodiments, the one or more synthetic oligosaccharides are or include one or more human milk oligosaccharides.

In some embodiments, the kits or articles of manufacture are or include one or more of 2′-fucosyllactose, 3-fucosyllactose, lacto-N-tetraose, or lacto-N-neotetraose, and at least one probiotic strain of B. longum subsp. infantis. In particular embodiments, the kits or articles of manufacture are or include at least two, at least three, or all four of 2′-fucosyllactose, 3-fucosyllactose, lacto-N-tetraose, or lacto-N-neotetraose. In certain embodiments, the kits or articles of manufacture include 2′-fucosyllactose and lacto-N-neotetraose.

In certain embodiments, the kits or articles of manufacture are or include one or more of 2′-fucosyllactose, 3-fucosyllactose, 3′-sialyllactose, 6′-sialyllactose, lacto-N-tetraose, lacto-N-neotetraose, or difucosyllactose and at least one probiotic strain of B. longum subsp. infantis. In particular embodiments, the kits or articles of manufacture are or include at least two, three, four, five, or all of 2′-fucosyllactose, 3-fucosyllactose, 3′-sialyllactose, 6′-sialyllactose, lacto-N-tetraose, lacto-N-neotetraose, or difucosyllactose and at least one probiotic strain of B. longum subsp. infantis.

In some embodiments, the kits or articles of manufacture are or include (i) a concentrated human milk permeate composition containing at least 10, 25, 50, or 100 human milk oligosaccharides, (ii) one or more synthetic human milk oligosaccharides, and (iii) at least one probiotic strain of B. longum subsp. infantis.

### III. CONDITIONS, DISEASES, AND DISORDERS

In certain embodiments, provided herein are methods for treating, preventing, or ameliorating one or more diseases, disorders, or conditions that are or may be associated with dysbiosis, e.g., of the intestinal microbiome, in a subject in need thereof. In certain embodiments, administration of the prebiotics, e.g., the concentrated human milk permeate composition and/or the synthetic oligosaccharides, and the probiotic strain, e.g., B. longum subsp. infantis, are useful to treat, ameliorate, remedy, or prevent diseases, disorders, or conditions such as obesity, inflammatory bowel disease (IBD), celiac disease, irritable bowel syndrome (IBS), colon cancer, diabetes, liver disorders, cystic fibrosis, and allergies.

In certain embodiments, the prebiotics, e.g., the concentrated human milk permeate composition and/or the synthetic oligosaccharides, and the at least one probiotic strain, e.g., B. longum subsp. infantis, are administered to a subject to treat, ameliorate, remedy, or prevent a gastrointestinal condition, disease, or disorder associated with, related to, or caused by dysbiosis, e.g., of the intestinal microbiome. In certain embodiments, the gastrointestinal condition, disease, or disorder is or includes one or more of a chronic inflammatory disease, an autoimmune disease, an infection, bowel resection, and/or a condition associated with chronic diarrhea. In certain embodiments, the gastrointestinal condition, disease, or disorder is or includes one or more of irritable bowel syndrome (IBS), inflammatory bowel disease (IBD) including Crohn's Disease and colitis, short bowel syndrome (SBS), celiac disease, small intestinal bacterial overgrowth (SIBO), gastroenteritis, leaky gut syndrome, and gastric lymphoma. In certain embodiments, the gastrointestinal condition, disease, or disorder is associated with a bacterial, viral, or parasitic infection or overgrowth. In a particular embodiment, the disease or disorder is associated with infection by drug-resistant bacteria, e.g., vancomycin-resistant enterococcus (VRE). In particular embodiments, administration of the prebiotics, e.g., the concentrated human milk permeate composition and/or the synthetic oligosaccharides, and the at least one probiotic strain, e.g., B. longum subsp. infantis, prevents, reduces, or ameliorates one or more symptoms of the gastrointestinal condition.

In some embodiments, the prebiotics, e.g., the concentrated human milk permeate composition and/or the synthetic oligosaccharides, and the at least one probiotic strain, e.g., B. longum subsp. infantis, are administered to a subject with an immune dysfunction. In some embodiments, the subject is immunocompromised. In certain embodiments, the administration prevents, reduces, treats, or ameliorates an infection in the immunocompromised subject. In some embodiments, the administration prevents, reduces, treats, or ameliorates overgrowth or domination of pathogenic bacteria. In some embodiments, the immunocompromised subject has undergone one or more treatments for cancer. In some embodiments, the treatments are or include chemotherapy. In certain embodiments, the treatment is or includes an allogenic transplant, e.g., a hematopoietic stem cell transplant or bone marrow transplant. In certain embodiments, the immunocompromised subject is in an ICU, has received an organ transplant, is elderly (e.g., at least 65 or 75 years old) and/or has been on prolonged antibiotic treatment (e.g., for at least 2, 3, 4, 6, 8, 10, or 12 weeks, or at least 1, 2, 3, 6, 12, 18, or 24 months). In certain embodiments, the administration prevents or reduces the probability or likelihood of a systemic infection by, by about, or by at least 10%, 20%, 30%, 40%, 50%, 60%, 70%, 75%, 80%, 90%, or 95%, e.g., as compared to a subject administered an alternative treatment and/or not administered the at least one probiotic strain and/or the prebiotics.

In particular embodiments, the prebiotics, e.g., the concentrated human milk permeate composition and/or the synthetic oligosaccharides, and the probiotic strain, are administered to treat or prevent overgrowth or domination of pathogenic bacteria (also referred to herein as gut domination). In some aspects, domination of pathogenic bacteria refers to the presence of a species of bacteria (e.g., a pathogenic species), of at least 1%, 5%, 10%, 20%, or 30%, relative to the bacteria present in the subject's gut or intestinal microbiome. Particular embodiments contemplate that overgrowth or domination may be determined by routine techniques in the art, such as including but not limited to PCR or high throughput sequencing.

In certain embodiments, the prebiotics, e.g., the concentrated human milk permeate composition and/or the synthetic oligosaccharides, and the at least one probiotic strain, e.g., B. longum subsp. infantis, are administered to a subject having, suspected of having, or at risk of having dysbiosis, e.g., of the intestinal microbiome. In certain embodiments, the transient presence, engraftment, or expansion of the probiotic strain, e.g., B. longum subsp. infantis, reduces, decreases, or ameliorates the dysbiosis. Particular embodiments contemplate that the presence, engraftment, or expansion of the probiotic strain, e.g., B. longum subsp. infantis, creates, promotes, or generates an environment and/or one or more conditions that (i) promotes the presence, growth, or expansion of beneficial microbiota; (ii) decreases the presence, growth, or expansion of pathogenic microbiota; (iii) promotes diversity of microbiota present within the microbiome; or (iv) any or all of (i) through (iii).

In certain embodiments, administration of the prebiotics, e.g., concentrated human milk permeate composition and/or the synthetic oligosaccharides, and at least one probiotic strain of bacterium, e.g., B. longum subsp. infantis, reduces the presence or abundance of pathogenic bacteria in the subject's gut. In certain embodiments, administration of the prebiotics and at least one probiotic strain of bacterium reduces gut domination by pathogenic taxa (e.g., Enterobacteriaceae, Enterococcus, Staphylococcus). In particular embodiments, the growth of the at least one probiotic strain, e.g., B. longum subsp. infantis, within the gut or microbiome reduces the abundance, level, activity, or presence of pathogenic taxa. In certain embodiments, administration of the prebiotics, e.g., the concentrated human milk permeate composition and/or the synthetic oligosaccharides, and the probiotic strain, e.g., B. longum subsp. infantis, reduces the abundance, level, activity, or presence of pathogenic bacteria and/or taxa by, by about, or by at least 10%, 20%, 30%, 40%, 50%, 60%, 70%, 75%, 80%, 90%, 95%, or 100%, e.g., as compared to prior to the administration or as compared to the gut or microbiome of a subject not administered the at least one probiotic strain and/or the prebiotics. In particular embodiments, the growth of the at least one probiotic strain, e.g., B. longum subsp. infantis, within the gut or microbiome increases the amount, level, presence, or concentration of at least one short chain fatty acid, e.g., acetate or butyrate, within the gut.

In certain embodiments, the prebiotics, e.g., the concentrated human milk permeate composition and/or the synthetic oligosaccharides, and the probiotic strain, e.g., B. longum subsp. infantis, are administered to a subject who is at risk of an infection or gut domination, e.g., by pathogenic bacteria. In some embodiments, the subject has an increased risk of infection or gut domination, e.g., as compared to the general population. In certain embodiments the subject is immunocompromised, undergoing an extended antibiotic treatment regimen (e.g., lasting at least 2, 3, 4, 5, 6, 8 10, or 12 weeks or 2, 3, 6, 12, 18, or 24 months), is elderly, is hospitalized e.g., in an intensive care unit (ICU), has received an organ transplant, and/or is immunosuppressed. In certain aspects, the subject will undergo or has received a medical procedure such a surgery or a chemotherapy that may increase the risk, likelihood, or probability of infection.

In certain embodiments, administration of the prebiotic strain and probiotics reduces the risk, likelihood, or probability of infection, e.g., by pathogenic bacteria, is reduced by at least 10%, 20%, 25%, 30%, 40%, 50%, 60%, 70%, 75%, 80%, 90%, 99%, or 99.9% as compared to alternative treatments or no treatments, or as compared to administration of the probiotic strain or prebiotics alone. In some embodiments, the prebiotics and the probiotic strain are administered at least once at least 1 hour, 2 hours, 3 hours, 4 hours, 6 hours, 8 hours, 12 hours, 18 hours, 24 hours, 1 day, 2 days, 3 days, 4 days, 5 days, 7 days, 10 days, 1 week, 2 weeks, 4 weeks, 6 weeks, one month, or two months prior to the medical procedure, e.g., surgery or chemotherapy. In particular embodiments, the prebiotics and the probiotic strain are administered at least once during the medical procedure, e.g., surgery or chemotherapy. In certain embodiments, the prebiotics and the probiotic strain are administered at least once at least 1 hour, 2 hours, 3 hours, 4 hours, 6 hours, 8 hours, 12 hours, 18 hours, 24 hours, 1 day, 2 days, 3 days, 4 days, 5 days, 7 days, 10 days, 1 week, 2 weeks, 4 weeks, 6 weeks, one month, or two months after to the medical procedure, e.g., surgery or chemotherapy.

Pathogenic bacteria may include known microbes with pathogenicity for the gastrointestinal tract, e.g., from esophagus down to rectum. In some embodiments, pathogenic bacteria are or include one or more species, subspecies, or strains of Proteobacteria. In certain embodiments, the pathogenic bacteria may include, but are not limited to strains, species, subspecies, or strains of one or more of Firmicutes, Clostridium, Enterobacteriaceae, Enterococcus, Staphylococcus, Corynebacteria, Salmonella, Shigella, Staphylococcus, Campylobacter (e.g., Campylobacter jejuni), Clostridia, Escherichia coli, Yersinia, Vibrio cholerae, Mycobacterium avium subspecies paratuberculosis, Brachyspira hyodysenteriae, or Lawsonia intracellularis. In some embodiments, the pathogenic bacteria may include, but are not limited to species, subspecies, or strains of Aeromonas, Bacillus, Bordetella, Borrelia, Brucella, Burkholderia, Campylobacter, Chlamydia, Chlamydophila, Citrobacter, Clostridium, Corynebacterium, Coxiella, Ehrlichia, Enterobacter, Enterobacteriaceae, Enterococcus, Escherichia, Francisella, Haemophilus, Helicobacter, Klebsiella, Legionella, Leptospira, Listeria, Morganella, Mycobacterium, Mycoplasma, Neisseria, Orientia, Plesiomonas, Proteus, Pseudomonas, Rickettsia, Salmonella, Shigella, Staphylococcus, Streptococcus, Treponema, Vibrio, or Yersinia. In particular embodiments, administration of the prebiotics and the at least one probiotic strain reduces or decreases the presence, growth, or abundance of pathogenic bacteria within the gut.

In some embodiments, administration of the prebiotics and the at least one probiotic strain impairs the growth of one or more pathogens. Such pathogens treated by the provided methods include, but are not limited to, Aeromonas hydrophila, Bacillus, e.g., Bacillus cereus, Bifidobacterium, Bordetella, Borrelia, Brucella, Burkholderia, C. difficile, Campylobacter, e.g., Campylobacter fetus and Campylobacter jejuni, Chlamydia, Chlamydophila, Clostridium, e.g., Clostridium botulinum, Clostridium difficile, and Clostridium perfringens, Corynebacterium, Coxiella, Ehrlichia, Enterobacteriaceae, e.g., Carbapenem-resistant Enterobacteriaceae (CRE) and Extended Spectrum Beta-Lactamase producing Enterobacteriaceae (ESBL-E), fluoroquinolone-resistant Enterobacteriaceae, Enterococcus, e.g., vancomycin-resistant Enterococcus spp., extended spectrum beta-lactam resistant Enterococci (ESBL), and vancomycin-resistant Enterococci (VRE), Escherichia, e.g., enteroaggregative Escherichia coli, enterohemorrhagic Escherichia coli, enteroinvasive Escherichia coli, enteropathogenic E. coli, enterotoxigenic Escherichia coli (such as but not limited to LT and/or ST), Escherichia coli 0157:H7, and multi-drug resistant bacteria E. coli, Francisella, Haemophilus, Helicobacter, e.g., Helicobacter pylori, Klebsiella, e.g., Klebsiellia pneumonia and multi-drug resistant bacteria Klebsiella, Legionella, Leptospira, Listeria, e.g., Lysteria monocytogenes, Morganella, Mycobacterium, Mycoplasma, Neisseria, Orientia, Plesiomonas shigelloides, Antibiotic-resistant Proteobacteria, Proteus, Pseudomonas, Rickettsia, Salmonella, e.g., Salmonella paratyphi, Salmonella spp., and Salmonella typhi, Shigella, e.g., Shigella spp., Staphylococcus, e.g., Staphylococcus aureus and Staphylococcus spp., Streptococcus, Treponema, Vibrio, e.g., Vibrio cholerae, Vibrio parahaemolyticus, Vibrio spp., and Vibrio vulnificus, and Yersinia, e.g., Yersinia enterocolitica. At least one of the one or more pathogens can be an antibiotic-resistant bacterium (ARB), e.g., Antibiotic-resistant Proteobacteria, Vancomycin Resistant Enterococcus (VRE), Carbapenem Resistant Enterobacteriaceae (CRE), fluoroquinolone-resistant Enterobacteriaceae, or Extended Spectrum Beta-Lactamase producing Enterobacteriaceae (ESBL-E).

In some embodiments, the condition, disease, or disorder is an immune dysfunction that is an autoimmune disorder. In some embodiments, the autoimmune disorder includes, but is not limited to, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosis, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's Disease), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & Ill autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

In some embodiments, the condition, disease, or disorder is a diarrheal disease including, but not limited to, acute bloody diarrhea (e.g., dysentery), acute watery diarrhea (e.g., cholera), checkpoint inhibitor-associated colitis, diarrhea due to food poisoning, persistent diarrhea, and traveler's diarrhea.

In some embodiments, administration of the at least one probiotic strain and the prebiotics treat or prevent various GI disorders known to result from or be associated or accompanied with dysbiosis of the intestinal microbiome. In certain embodiments, administration of the at least on probiotic strain and prebiotics reduces GI immunoactivation and/or inflammation. In some embodiments, GI immuno-activation and inflammation may be assessed by known methods that are routine in the art. In some embodiments, the condition, disease, or disorder is an inflammatory bowel disease (IBD) or related disease including, but not limited to, Behcet's disease, collagenous colitis, Crohn's disease, diversion colitis, fulminant colitis, intermediate colitis, left-sided colitis, lymphocytic colitis, pancolitis, pouchitis, proctosigmoiditis, short bowel syndrome, ulcerative colitis, and ulcerative proctitis.

In various embodiments, administration of the at least one probiotic strain and the prebiotics treats or prevents various bloodstream infections (BSI). In certain embodiments, administration of the probiotic strain and the prebiotics treats or prevents catheter or intravascular-line infections (e.g., central-line infections). In some embodiments, administration of the probiotic strain and the prebiotics treats or prevents chronic inflammatory diseases.

In particular embodiments, administration of the at least one probiotic strain and the prebiotics treats or prevents meningitis; pneumonia, e.g., ventilator-associated pneumonia; skin and soft tissue infections; surgical-site infections; urinary tract infections (e.g., antibiotic-resistant urinary tract infections and catheter-associated urinary tract infections); wound infections; and/or antibiotic-resistant infections and antibiotic-sensitive infections.

In certain embodiments, administration of the at least one probiotic strain and the prebiotics treats or prevents diseases or disorders relating to the “gut-brain axis”, including neurodegenerative, neurodevelopmental, and neurocognitive disorders, such as anorexia, anxiety, autism-spectrum disorder, depression, Parkinson's, and Schizophrenia. In certain embodiments, administration of the at least one probiotic strain and the prebiotics reduces one or more symptoms associated with anorexia, anxiety, autism-spectrum disorder, depression, Parkinson's, and/or Schizophrenia.

In some embodiments, administration of the at least one probiotic strain and the prebiotics treats or prevents a side effect of an anti-cancer therapy and/or increases efficacy of an anti-cancer therapeutic agent and/or anti-cancer therapy. In some embodiments, the anti-cancer therapy is surgery, radiation therapy, chemotherapy (including hormonal therapy) and/or targeted therapy (including an immunotherapy). Illustrative chemotherapeutics agents are provided elsewhere herein. In particular embodiments, the immunotherapy binds to and/or recognizes a tumor-cell antigen and/or a cancer-cell antigen, e.g., CTLA-4, PD-1, PD-L1, or PD-L2. In some embodiments, the immunotherapy comprises administration of Keytruda (Pembrolizumab), Opdivo (Nivolumab), Yervoy (Ipilimumab), Tecentriq (atezolizumab), Bavencio (avelumab), and Imfinzi (durvalumab).

In some embodiments, the subject is refractory and/or non-responsive to an anti-cancer therapy. In certain embodiments, the probiotic strain and prebiotics treats a subject that presents a non-curative response, a limited response, or no response to the anti-cancer therapy, or even progress, after 12 weeks or so of receiving the anti-cancer therapy. Thus, in some aspects, the provided probiotic strain and prebiotics of the present invention can rescue subjects that are refractory and/or non-responsive to the anti-cancer therapy. In certain embodiments, the subject is refractory and/or non-responsive to a treatment directed to a checkpoint molecule, e.g., CTLA-4, PD-1, PD-L1, and/or PD-L2. In particular embodiments, the treatment directed to a checkpoint molecule comprises administration of Keytruda (Pembrolizumab), Opdivo (Nivolumab), Yervoy (Ipilimumab), Tecentriq (atezolizumab), Bavencio (avelumab), or lmfinzi (durvalumab).

In some embodiments, the prebiotics, e.g., concentrated human milk permeate composition and/or the synthetic oligosaccharides, and the at least one probiotic strain, e.g., B. longum subsp. infantis, are administered to an immunocompromised subject. In certain embodiments, the administration prevents, reduces, treats, or ameliorates an infection in the immunocompromised subject. In some embodiments, the administration prevents, reduces, treats, or ameliorates overgrowth or domination of pathogenic bacteria. In some embodiments, the immunocompromised subject has undergone one or more treatments for cancer. In some embodiments, the treatments are or include chemotherapy. In certain embodiments, the treatment is or includes an allogenic transplant, e.g., a hematopoietic stem cell transplant or bone marrow transplant. In certain embodiments, the administration prevents or reduces the probability or likelihood of a systemic infection by, by about, or by at least 10%, 20%, 30%, 40%, 50%, 60%, 70%, 75%, 80%, 90%, or 95%, e.g., as compared to an alternative treatment or treatment with either the probiotic strain or prebiotics alone.

In certain embodiments, the prebiotics, e.g., the concentrated human milk permeate composition and/or the synthetic oligosaccharides, and the at least one probiotic strain, e.g., B. longum subsp. infantis, are administered to a subject who has or is at risk of sepsis. In some embodiments, the probability or likelihood of sepsis is reduced or decreased by at least 10%, 20%, 30%, 40%, 50%, 60%, 70%, 80%, 90%, 95%, or 99%, e.g., as compared to a subject (e.g., who has or is at risk for sepsis) not administered the prebiotics or the at least one probiotic. In certain embodiments, the administration of the prebiotics and the at least one probiotic improves or increases the survival of the subject over 6 months, 12 months, 18 months, 1 year, 2 years, 5 years, 10 years, and/or 20 years or more by, by about, or by at least 5%, 10%, 20%, 25%, 30%, 40%, 50%, 60%, 70%, 75%, 80%, 90%, 95%, 100%, or 1-fold, 2-fold, 3-fold, 4-fold, or 5-fold greater than in subjects (e.g., who have or are at risk for sepsis) not administered the prebiotics and the at least one probiotic strain.

In particular embodiments, administration of the prebiotics and the at least one probiotic strain prevents, reduces, decreases, remedies, or ameliorates one or more symptoms associated with a gastrointestinal condition, disease, or disorder. In certain embodiments, the one or more symptoms associated with gastrointestinal condition, disease, or disorder may include, but are not limited to, diarrhea, fever, fatigue, abdominal pain and cramping, blood in stool, mouth sores, weight loss, fistula, inflammation (of skin, eyes, or joints), inflamed liver or bile ducts, delayed growth (in children). In particular embodiments, administration of the prebiotics and the at least one probiotic strain reduces the risk or probability for the subject of experiencing one or more symptoms associated with the gastrointestinal condition, disease, or disorder by, by about, or by at least 10%, 20%, 30%, 40%, 50%, 60%, 70%, 75%, 80%, 90%, or 95%, e.g., as compared to a subject not administered the at least one probiotic strain and/or the prebiotics. In certain embodiments, administration of the prebiotics and the at least one probiotic strain increases probability or likelihood for remission by, by about, or by at least 10%, 20%, 30%, 40%, 50%, 60%, 70%, 75%, 80%, 90%, 100%, or 1-fold, 2-fold, 5-fold, 10-fold, 20-fold, 50-fold, or 100-fold e.g., as compared to a subject not administered the at least one probiotic strain and/or the prebiotics. In some embodiments, administration of the prebiotics and the at least one probiotic strain increases probability or likelihood for remission within 12 weeks, 10 weeks, 8 weeks, 6 weeks, 4 weeks, or less than 4 weeks, e.g., from the initiation or termination of the administration.

In various embodiments, the prebiotics, e.g., the concentrated human milk permeate composition and/or the synthetic oligosaccharides, and the at least one probiotic strain, e.g., B. longum subsp. infantis, are administered to a subject to treat, ameliorate, remedy, or prevent a chronic inflammatory disease, an autoimmune disease, an infection, bowel resection, and/or a condition associated with chronic diarrhea. According to particular embodiments, the pathology is selected from the group consisting of: irritable bowel syndrome (IBS), inflammatory bowel disease (IBD), short bowel syndrome (SBS), celiac disease, small intestinal bacterial overgrowth (SIBO), gastroenteritis, leaky gut syndrome, and gastric lymphoma. In another embodiment the disease or disorder is associated with a bacterial, viral, or parasitic infection or overgrowth, e.g. by drug-resistant bacteria. In some embodiments, administration of the prebiotics and the at least one probiotic strain increases probability or likelihood for cure or remission of the chronic inflammatory disease, autoimmune disease, infection, bowel resection, and/or chronic diarrhea for by, by about, or by at least 10%, 20%, 30%, 40%, 50%, 60%, 70%, 75%, 80%, 90%, 100%, or 1-fold, 2-fold, 5-fold, 10-fold, 20-fold, 50-fold, or 100-fold e.g., as compared to a subject not administered the at least one probiotic strain and/or the prebiotics. In some embodiments, administration of the prebiotics and the at least one probiotic strain increases probability or likelihood for the cure or remission within 12 weeks, 10 weeks, 8 weeks, 6 weeks, 4 weeks, or less than 4 weeks, e.g., from the initiation or termination of the administration.

In certain embodiments, the probiotic strain and the prebiotics are administered to a subject to treat, prevent, or ameliorate an allergy. In some embodiments, the allergy is a food allergy. In certain embodiments, the food allergy is or includes a chronic or acute immunological hypersensitivity reaction (e.g. a type I hypersensitivity reaction) elicited in a mammal in response to an ingested material or food antigen (also referred to in the art as a “food allergen”). Identification and diagnosis of food allergy is routine among persons of ordinary skill in the art. Food allergies may include, but are not limited to, allergies to nuts, peanuts, shellfish, fish, milk, eggs, wheat, or soybeans.

In some embodiments, the probiotic strain and the prebiotics are administered to treat or ameliorate an allergy, e.g., a food allergy. In certain embodiments, the probiotic strain and the prebiotics reduce or decrease the severity of the allergic response to the allergen, e.g., as compared to the allergic response prior to any treatment with the probiotic strain and prebiotics. In certain embodiments, the probiotic strain and the prebiotics attenuates or reduces the severity or intensity of one or more symptoms or clinical manifestations of the allergy, e.g., food allergy, to subsequent exposures to the allergen, e.g., as compared to symptoms or clinical manifestations observed prior to treatment with the probiotic strain and prebiotics. In some embodiments, the symptoms or clinical manifestations of the allergy may include, but are not limited to rash, eczema, atopic dermatitis, hives, urticaria, angioedema, asthma, rhinitis, wheezing, sneezing, dyspnea, swelling of the airways, shortness of breath, other respiratory symptoms, abdominal pain, cramping, nausea, vomiting, diarrhea, melena, tachycardia, hypotension, syncope, seizures, and anaphylactic shock.

In particular embodiments, the probiotic strain and the prebiotics are administered to a subject, e.g., a subject at risk of having or developing an allergy, to prevent or reduce or decrease the probability or likelihood experiencing an allergic response. In certain embodiments, administration of the probiotic strain and prebiotics reduce the likelihood or probability of having an allergic response within the next month, 3 months, 6 months, 12 months, 18 months, year, 2 years, 3 years, 5 years, 10 years, or 20 years. In some embodiments, the probability or likelihood of developing the allergy is reduced by at least 10%, 20%, 30%, 40%, 50%, 60%, 70%, 75%, 80%, 90%, or 99% as compared to a subject with a similar risk profile who is not administered the probiotic strain and the prebiotics. In some embodiments, administration of the probiotic strain and prebiotics reduces the severity of one or more symptoms or clinical manifestations of an allergic response following exposure to the allergen over the next month, 3 months, 6 months, 12 months, 18 months, year, 2 years, 3 years, 5 years, 10 years, or 20 years, e.g., as compared to exposure of the allergen to a subject with the same or similar allergy who was not administered the probiotic strain and the prebiotics.

In some embodiments, the prebiotics, e.g., the concentrated human milk permeate composition and/or the synthetic oligosaccharides, and the at least one probiotic strain, e.g., B. longum subsp. infantis, are administered to a subject to treat, ameliorate, remedy, or prevent pouchitis. In certain aspects, pouchitis is inflammation that occurs in the lining of a pouch created during surgery to treat ulcerative colitis or certain other diseases. In some embodiments, the surgery is or includes removal of a diseased colon or portion thereof. In certain embodiments, the surgery is a J pouch surgery (ileoanal anastomosis IPAA).

In some embodiments, the prebiotics, e.g., the concentrated human milk permeate composition and/or the synthetic oligosaccharides, and the at least one probiotic strain, e.g., B. longum subsp. infantis, are administered to a subject to treat, ameliorate, remedy, or prevent pouchitis in a subject in need thereof, e.g., a subject who has undergone an IPAA surgery. In particular embodiments, administration of the prebiotics and the at least one probiotic strain prevents, reduces, decreases, remedies, or ameliorates one or more symptoms associated with pouchitis. In certain embodiments, the one or more symptoms associated with pouchitis may include, but are not limited to, increased stool frequency, tenesmus, straining during defecation, blood in the stool, incontinence, seepage of waste matter during sleep, abdominal cramps, pelvic or abdominal discomfort, or tail bone pain. In certain embodiments, symptoms associated with more severe pouchitis include, but are not limited to, fever, dehydration, malnutrition, fatigue, iron-deficiency anemia, or joint pain. In particular embodiments, administration of the prebiotics and the at least one probiotic strain reduces the risk or probability for the subject of experiencing pouchitis by, by about, or by at least 10%, 20%, 30%, 40%, 50%, 60%, 70%, 75%, 80%, 90%, or 95%, e.g., as compared to a subject not administered the at least one probiotic strain and/or the prebiotics.

In various embodiments, the prebiotics, e.g., of human milk oligosaccharides, and the at least one probiotic strain, e.g., B. longum subsp. infantis, are administered to a subject to treat, ameliorate, remedy, or prevent a chronic inflammatory disease, an autoimmune disease, an infection, bowel resection, and/or a condition associated with chronic diarrhea. Such pathology includes, but is not limited to: irritable bowel syndrome (IBS), inflammatory bowel disease (IBD), short bowel syndrome (SBS), celiac disease, small intestinal bacterial overgrowth (SIBO), gastroenteritis, leaky gut syndrome, and gastric lymphoma. In some embodiments the disease or disorder is associated with a bacterial, viral, or parasitic infection or overgrowth, e.g. by drug-resistant bacteria. In some embodiments, administration of the prebiotics and the probiotic strain increases probability or likelihood for cure or remission of the chronic inflammatory disease, autoimmune disease, infection, bowel resection, and/or chronic diarrhea for by, by about, or by at least 10%, 20%, 30%, 40%, 50%, 60%, 70%, 75%, 80%, 90%, 100%, or 1-fold, 2-fold, 5-fold, 10-fold, 20-fold, 50-fold, or 100-fold e.g., as compared to a subject not administered the probiotic strain and prebiotics and/or a subject administered an alternative therapy. In some embodiments, administration of the prebiotics and the probiotic strain increases probability or likelihood for the cure or remission within 12 weeks, 10 weeks, 8 weeks, 6 weeks, 4 weeks, or less than 4 weeks, e.g., from the initiation or termination of the administration e.g., as compared to a subject not administered the probiotic strain and prebiotics and/or a subject administered an alternative therapy.

In some embodiments, the subject is a patient in an intensive care unit (ICU). In some embodiments, the subject is an organ transplant recipient. In some embodiments, the subject is a geriatric patient (e.g., at least 65, 70, 75, 80, or 85 years old). In some embodiments, the subject has received prolonged antibiotic treatment (e.g., at least 2, 3, 4, 5, 6, 8, 10, or 12 weeks, or at least 1, 2, 3, 6, 12, 18, or 24 months). In some embodiments, the subject is a recipient of a broad-spectrum antibiotic treatment. In some embodiments, the subject is a recipient, or recent recipient (e.g., within at least 1, 2, 3, 4, 5, 6, or 7 days, or within at least 1, 2, 3, or 4 weeks), of parenteral nutrition (e.g., total parenteral nutrition or partial parenteral nutrition). In some embodiments, the subject is a recipient of enteral nutrition.

### A.) GVHD

In particular embodiments, provided herein are methods of preventing or reducing the risk, incidence, and/or the severity of graft versus host disease (GVHD) in a subject in need thereof. In certain embodiments, the provided methods prevent or reduce incidence or severity of GVHD in a subject that has received or will receive an allogenic stem cell transplant. In some embodiments, the provided prebiotics are formulated to be administered to subjects who have, are, or will undergo an allogenic transplant, e.g., BMT or HSCT. In some embodiments, the at least one probiotic stain is formulated to be administered to subject who has underwent, is undergoing, or will undergo an allogenic transplant. In certain embodiments, the method is or includes steps for administering to the subject the prebiotics, such as any described herein e.g., in Section II-A or Section II-B, and at least one probiotic strain, such as a probiotic strain described herein, e.g., in Section II-C or listed in Table 1. In particular embodiments, the at least one probiotic strain and the prebiotics are administered by any of the methods and/or treatment regimens as described herein, e.g., in Section I.

In certain embodiments, the method is or includes administering prebiotics, e.g., the concentrated human milk permeate composition and/or the synthetic oligosaccharides, and at least one probiotic strain, e.g., B. longum subsp. infantis. In some embodiments, the method includes administering the concentrated human milk permeate composition and/or the synthetic oligosaccharides, such as any of those that are described herein, e.g., in Section II-A or II-B, and administering at least one probiotic strain, e.g., a Bifidobacterium, such as any one or more of those described herein, e.g., in Section II-C. In certain embodiments, the concentrated human milk permeate composition, the one or more synthetic oligosaccharides, and the at least one probiotic strain, e.g., B. longum subsp. infantis, are administered separately, such as at different times or in separate compositions, formulations, or doses. In particular embodiments, the concentrated human milk permeate composition, the one or more synthetic oligosaccharides, and the at least one probiotic strain, e.g., B. longum subsp. infantis, are administered together, such as at the same time or in the same composition, formulation, or dose.

In certain embodiments, the prebiotics, e.g., the concentrated human milk permeate composition and/or the one or more synthetic oligosaccharides, are administered to treat, prevent, ameliorate, reduce, or decrease GVHD in a subject in need thereof. In some embodiments, the probiotic strain and the prebiotics are administered to treat, prevent, ameliorate, reduce, or decrease GVHD in a subject in need thereof. In certain embodiments, the subject is a mammal. In particular embodiments, the subject is a human. In certain embodiments, the subject is a human infant, child, adolescent, or adult. In particular embodiments, the subject is at risk or suspected of being at risk of having GVHD. In some embodiments, the GHVD is associated with, or accompanied by, an allogenic transplant, such as an allogenic bone marrow transplant (BMT) or an allogenic hematopoietic stem cell transplant (allo-HSCT).

In some embodiments, the prebiotics and the at least one probiotic strain are administered to a subject that has undergone or will undergo an allogenic stem cell transplant. In certain embodiments, the allogenic transplant is a bone marrow transplant (BMT). In particular embodiments, the allogenic transplant is a hematopoietic stem cell transplantation (HSCT). In particular embodiments, the subject has undergone the allogenic stem cell transplant within 12 weeks, 8 weeks, 6 weeks, 4 weeks, 3 weeks, 2 weeks, 14 days, 12 days 10 days, 7 days, 5 days, 4 days, 3 days, 2 days, or 1 day prior to administration of a first dose of the prebiotics or the at least one probiotic strain. In certain embodiments, the first dose of the prebiotics or the at least one probiotic strain is administered within 12 weeks, 8 weeks, 6 weeks, 4 weeks, 3 weeks, 2 weeks, 14 days, 12 days 10 days, 7 days, 5 days, 4 days, 3 days, 2 days, or 1 day prior to receiving the allogenic stem cell transplant.

In some embodiments, provided herein are methods for treating, preventing, or ameliorating GVHD in a subject in need thereof. In certain embodiments, provided herein are methods for treating, preventing, or ameliorating a condition or disease associated or accompanied with GVHD in a subject in need thereof. In certain embodiments, provided herein are methods for treating, preventing, reducing, decreasing, or ameliorating the severity or presence of one or more symptoms associated with GVHD or a disease or condition associated or accompanied with GVHD in a subject in need thereof.

In particular embodiments, administration of the prebiotics and the at least one probiotic strain reduces or decreases the probability or likelihood of experiencing GVHD. In certain embodiments, the probability or likelihood is reduced or decreased by at least 10%, 20%, 30%, 40%, 50%, 60%, 70%, 80%, 90%, 95%, or 99%, e.g., as compared to a subject not administered the prebiotics or the at least one probiotic. In certain embodiments, the probability or likelihood of experiencing GVHD within 20 years, 10 years, 7 years, 5 years, 2 years or 1 year, or within the subject's lifetime, is reduced or decreased, e.g., as compared to a subject not administered the prebiotics or the at least one probiotic.

In certain embodiments, the prebiotics, e.g., the concentrated human milk permeate composition and/or the one or more synthetic oligosaccharides, and the at least one probiotic strain, e.g., B. longum subsp. infantis, are administered to decrease or reduce mortality associated with an allogenic transplant, e.g., BMT or HSCT, or with GVHD. In some embodiments, the prebiotics and the probiotic strain are administered to increase survival of subjects who undergo an allogenic transplant, e.g., BMT or HSCT. In particular embodiments, administration of the prebiotics and the probiotic strain improves or increases the survival of the subject over 6 months, 12 months, 18 months, 1 year, 2 years, 5 years, 10 years, and/or 20 years or more by, by about, or by at least 5%, 10%, 20%, 25%, 30%, 40%, 50%, 60%, 70%, 75%, 80%, 90%, 95%, 100%, or 1-fold, 2-fold, 3-fold, 4-fold, or 5-fold greater than in subjects (e.g., subjects who received an allogenic transplant, e.g., BMT or HSCT) not administered the prebiotics and the at least one probiotic strain.

In some embodiments, the prebiotics, e.g., the concentrated human milk permeate composition and/or the one or more synthetic oligosaccharides, and the at least one probiotic strain, e.g., B. longum subsp. infantis, are administered to treat, prevent, ameliorate, reduce, or decrease the severity, occurrence, or likelihood of experiencing one or more symptoms, e.g., symptoms associated with or accompanying GHVD. In particular embodiments, the GVHD is acute GVHD. In some embodiments, the GVHD is chronic GVHD. In particular embodiments, the symptoms of GVHD are or include, but are not limited to, a rash, such as with burning or itching sensation; blistering, e.g., of the skin; flaking of the skin; nausea; vomiting; abdominal cramps; loss of appetite; diarrhea; and jaundice. In some embodiments, the symptoms of GVHD are or include, but are not limited to dry mouth, mouth ulcers, difficulty eating, gum disease, tooth decay, rash, itchy sensation, thickening and tightening of the skin, jaundice, changes in skin coloration, hair loss, premature gray hair, loss of body hair, loss of appetite, unexplained weight loss, nausea, vomiting diarrhea, stomach pain, shortness of breath, difficulty breathing, persistent or chronic cough, wheezing, impaired liver function, abdominal swelling, muscle weakness, muscle cramps, and joint stiffness. In particular embodiments, administration of the prebiotics and the at least one probiotic strain, e.g., B. longum subsp. infantis, treats, prevents, ameliorates, reduces, or decreases the severity, occurrence, or likelihood of the one or more symptoms as compared to what is observed in subjects (e.g., subjects who have had or will undergo an allogeneic transplant) that are not administered the prebiotics and/or probiotic strain. In some aspects, the presence, occurrence, and severity of a symptom may be recognized, identified, or scored by skilled person (e.g., a healthcare practitioner) as a matter of routine.

In certain embodiments, the provided methods are or include administering the concentrated human milk permeate composition, the one or more synthetic oligosaccharides, and at least one probiotic strain to a subject who will undergo or who has undergone an allogenic transplant, e.g., a BMT or HSCT. In particular embodiments, the concentrated human milk permeate composition is obtained from a permeate resulting from the ultra-filtration of skim from pooled human milk (such as described herein or produced by a method described herein, e.g., in Section II-A-(i)). In certain embodiments, the concentrated human milk permeate composition is or incudes at least 10, 25, 50, or 80 different human milk oligosaccharides. In certain embodiments, the one or more synthetic oligosaccharides are one or more synthetic human milk oligosaccharides. In certain aspects, the at least one probiotic strain is a Bifidobacterium. In particular embodiments, the at least one probiotic strain is B. longum subsp. infantis. In certain embodiments, administration of a concentrated human milk permeate composition, one or more synthetic human milk oligosaccharides, and a probiotic strain of B. longum subsp. infantis reduces or decreases the probability or likelihood of experiencing GVHD. In some embodiments, administration of a concentrated human milk permeate composition, one or more synthetic human milk oligosaccharides, and a probiotic strain of B. longum subsp. infantis reduces the incidence or severity of one or more symptoms associated with GVHD.

### B.) Solid Organ Transplant

In certain embodiments, provided herein are methods of preventing or reducing the risk, incidence, and/or the likelihood of rejection, e.g., an acute immune rejection, in a subject that has received or will receive a solid organ transplant. In some embodiments, the at least one probiotic stain, e.g., B. longum subsp. infantis, is formulated to be administered to subject who has underwent, is undergoing, or will undergo an allogenic transplant. In certain embodiments, the method is or includes steps for administering to the subject the prebiotics, such as any described herein e.g., in Section II-A or Section II-B, and at least one probiotic strain, such as a probiotic strain described herein, e.g., in Section II-C or listed in Table 1. In certain embodiments, the method is or includes administering prebiotics, e.g., the concentrated human milk permeate composition and/or the synthetic oligosaccharides, and at least one probiotic strain, e.g., B. longum subsp. infantis, to a subject that has received or will receive a solid organ transplant. In particular embodiments, the at least one probiotic and the prebiotics are administered as described herein, e.g., in Section I.

In some embodiments, the method includes administering the concentrated human milk permeate composition and/or the synthetic oligosaccharides, such as any of those that are described herein, e.g., in Section II-A or II-B, and administering at least one probiotic strain, e.g., a Bifidobacterium, such as any one or more of those described herein, e.g., in Section II-C. In certain embodiments, the concentrated human milk permeate composition, the one or more synthetic oligosaccharides, and the at least one probiotic strain, e.g., B. longum subsp. infantis, are administered separately, such as at different times or in separate compositions, formulations, or doses. In particular embodiments, the concentrated human milk permeate composition, the one or more synthetic oligosaccharides, and the at least one probiotic strain, e.g., B. longum subsp. infantis, are administered together, such as at the same time or in the same composition, formulation, or dose.

In some aspects, a solid organ transplant is an effective treatment for patients with end-stage dysfunction of kidney, liver, heart, lung, pancreas, or intestine. However, within the first year after transplant, up to 25% of recipients suffer from episodes of acute immune rejection. In certain aspects, many transplant recipients also receive immunosuppressive drugs as part of the transplantation procedure to prevent immediate rejection of the new organ (80% of patients for kidney, heart, lung, pancreas, intestine; 30% for liver), and roughly 90% of transplant recipients leave the hospital with a prescription for costly immunosuppressive drugs. Given that maintenance therapy with immunosuppressive drugs leads to increased risk of infection, solid organ transplant patients also receive prophylactic antibiotic therapy prior to transplant and may receive antibiotics post-transplant. Although immunosuppressive drugs and antibiotics are intended to broadly prevent rejection and infection, emerging evidence suggests that disruptive impacts of these drugs on the patient microbiome may in fact contribute to rejection or infection in the long-term by inducing microbiome dysbiosis. Thus, developing clinical interventions to restore and protect the microbiome in solid organ transplant recipients, including those receiving immunosuppressive drugs or antibiotics, is critical to improving outcomes and quality of life in this patient population. Particular embodiments of the provided methods and compositions address these needs.

In some embodiments, the prebiotics, e.g., the concentrated human milk permeate composition and/or the one or more synthetic oligosaccharides, and the at least one probiotic strain, e.g., B. longum subsp. infantis, are administered to reduce or decrease the risk or likelihood of rejection, e.g., acute immune rejection, in a subject who has or who will receive a solid organ transplant. In certain embodiments, the subject has or will receive a kidney, heart, lung, pancreas, intestine, or liver transplant. In particular embodiments, the subject has undergone the solid organ transplant within 12 weeks, 8 weeks, 6 weeks, 4 weeks, 3 weeks, 2 weeks, 14 days, 12 days 10 days, 7 days, 5 days, 4 days, 3 days, 2 days, or 1 day prior to administration of a first dose of the prebiotics or the at least one probiotic strain. In certain embodiments, the first dose of the prebiotics or the at least one probiotic strain is administered within 12 weeks, 8 weeks, 6 weeks, 4 weeks, 3 weeks, 2 weeks, 14 days, 12 days 10 days, 7 days, 5 days, 4 days, 3 days, 2 days, or 1 day prior to receiving the solid organ transplant.

In particular embodiments, administration of the prebiotics and the at least one probiotic strain, e.g., as described herein such as in Section I, reduces or decreases the probability or likelihood of experiencing rejection, e.g., as compared to a subject not administered the at least one probiotic strain and/or the prebiotics. In certain embodiments, the probability or likelihood is reduced or decreased by at least 10%, 20%, 30%, 40%, 50%, 60%, 70%, 80%, 90%, 95%, or 99%, e.g., as compared to a subject not administered the prebiotics and/or the at least one probiotic. In certain embodiments, the probability or likelihood of experiencing rejection within 1 year, 2 years, 5 years, 10 years, 20 years, or within the subject's lifetime, is reduced or decreased, e.g., as compared to a subject not administered the prebiotics or the at least one probiotic.

In certain embodiments, the prebiotics, e.g., the concentrated human milk permeate composition and/or the one or more synthetic oligosaccharides, and the at least one probiotic strain, e.g., B. longum subsp. infantis, are administered to decrease or reduce mortality associated with a solid organ transplant. In some embodiments, the prebiotics and the probiotic strain are administered to increase survival of subjects who undergo a solid organ transplant. In particular embodiments, administration of the prebiotics and the probiotic strain improves or increases the survival of the subject over 6 months, 12 months, 18 months, 1 year, 2 years, 5 years, 10 years, and/or 20 years or more by, by about, or by at least 5%, 10%, 20%, 25%, 30%, 40%, 50%, 60%, 70%, 75%, 80%, 90%, 95%, 100%, or 1-fold, 2-fold, 3-fold, 4-fold, or 5-fold greater than in subjects (e.g., subjects who received a solid organ transplant) that were not administered the prebiotics and/or the at least one probiotic strain.

### C.) Hyperammonemia

Also provided herein are compositions, methods, kits, and articles of manufacture that are useful, inter alia, in the treatment or prevention of hyperammonemia or related conditions and disorders in subjects in need thereof. In some aspects, provided herein is a mixture of human milk oligosaccharides with a low nitrogen content. In some embodiments, some, most, or all of the human milk oligosaccharides of the prebiotic mixture lack or do not incorporate one or more nitrogen containing residues e.g., an N-acetyl glucosamine residue, or chemical groups, e.g., an N-acetyl group. In certain aspects, the low nitrogen containing human milk oligosaccharides are administered with a probiotic strain of bacterium described herein, e.g., a Bifidobacterium such as B. longum subsp. infantis, capable of consuming or metabolizing human milk oligosaccharides. In certain embodiments, one or both of the at least one probiotic strain of bacterium and the human milk oligosaccharides are administered to a subject to treat, mend, remedy, ameliorate, or prevent hyperammonemia or one or more symptoms associated with hyperammonemia. In certain embodiments, the at least one probiotic strain is capable of internalizing the human milk oligosaccharides prior to consuming or metabolizing.

Ammonia is highly toxic and generated during metabolism. In mammals, the healthy liver protects the body from accumulating excess ammonia by converting it to non-toxic molecules, e.g., urea or glutamine, and preventing excess amounts of ammonia from entering the systemic circulation. Hyperammonemia is characterized by the decreased detoxification and/or increased production of ammonia. In healthy individuals, the urea cycle detoxifies ammonia by enzymatically converting ammonia into urea, which is then removed in the urine. Decreased ammonia detoxification may be caused by urea cycle disorders (UCDs) in which urea cycle enzymes are defective, such as argininosuccinic aciduria, arginase deficiency, carbamoylphosphate synthetase deficiency, citrullinemia, N-acetylglutamate synthetase deficiency, and ornithine transcarbamylase deficiency. In addition, several non-UCD disorders, such as hepatic encephalopathy, portosystemic shunting, and organic acid disorders, can also cause hyperammonemia. Hyperammonemia can produce neurological manifestations, e.g., seizures, ataxia, stroke-like lesions, coma, psychosis, vision loss, acute encephalopathy, cerebral edema, as well as vomiting, respiratory alkalosis, hypothermia, or death. Other conditions where elevated blood or serum levels of ammonia may be detected include autism spectrum disorder.

Current therapies for hyperammonemia, and associated conditions or diseases such as hepatic encephalopathy and UCDs, aim to reduce excess ammonia, but are widely regarded as suboptimal. For example, hepatic encephalopathy is associated with impairment of normal cognitive function due to confusion and memory loss, which can make it extremely difficult for individuals with hepatic encephalopathy to carry out repeated complex tasks such as preparing and timely taking their therapeutic agent regimen. Thus, it can be extremely difficult for individuals suffering from hyperammonemia to carry out the various tasks required of compliance including preparing the therapeutic agents (i.e. mixing solutions) and remembering to take the therapeutic agents. Furthermore, the currently available treatments such as lactulose and Rifaximin can have side effects which patients may find unbearable, such as including but not limited to diarrhea, nausea, vomiting, gas, stomach pain and abdominal discomfort. In addition, lactulose, a traditional therapeutic, is unpalatable to most individuals. As such, compliance of patients with hyperammonemia, e.g., hepatic encephalopathy patients, with therapeutic regiment is relatively poor. Thus, there is significant unmet need for effective, reliable, and/or long-term treatment for disorders associated with hyperammonemia, including hepatic encephalopathy.

In some embodiments, compositions, methods, techniques, kits, and articles of manufacture are provided that address these needs. In some aspects, administration of human milk oligosaccharides lacking or containing low amounts of nitrogen in conjunction with the probiotic strain, e.g., B. longum subsp. infantis, efficiently reduces the levels of ammonia, and in particular aspects demonstrate an improved reduction of ammonia as compared to known existing treatments. In certain aspects, the human milk oligosaccharides lacking or containing low nitrogen, reduces the amount or level of ammonia in a subject with less or even none of the unwanted side effects that may accompany the known alternative treatments. Thus, the compositions, methods, techniques, kits, and articles of manufacture of the invention provide improved treatments for hyperammonemia and associated conditions such as hepatic encephalopathy or UCD.

In some embodiments, the engraftment, growth, or expansion of the probiotic strain, e.g., B. longum subsp. infantis, reduces the amount, level, or presence of bacteria that produce ammonia, such as Enterobacteriaceae and other species, subspecies, or strains of bacteria with urease activity. In certain embodiments, the probiotic strain is any one or more of the probiotic strains described herein, such as in Section II-C. In certain embodiments, the probiotic strain is or includes B. longum subsp. infantis.

In some embodiments, compositions and methods useful for the treatment or prevention of hyperammonemia are described in PCT. App. No. PCT/US2020/052501, hereby incorporated by reference in its entirety.

In certain embodiments, provided herein is a composition or kit comprising one or more of 2′-fucosyllactose, 3-fucosyllactose, Lacto-N-tetraose, or Lacto-N-neotetraose and a strain of B. longum subsp. infantis. In particular embodiments, provided herein is a kit or composition containing 2′-fucosyllactose, 3-fucosyllactose, Lacto-N-tetraose, and Lacto-N-neotetraose, and at least one strain of B. longum subsp. infantis.

In some embodiments, the composition or the compositions within the kit are used in the manufacture of a medicament for the treatment or prevention of hyperammonemia or to reduce ammonia in a subject in need thereof.

In certain embodiments, provided herein is a method of treating hyperammonemia comprising administering to a subject in need thereof one or more of one or more of 2′-fucosyllactose, 3-fucosyllactose, Lacto-N-tetraose, or Lacto-N-neotetraose and B. longum subsp. infantis. In particular embodiments, the human milk oligosaccharides that are not 2′-fucosyllactose, 3-fucosyllactose, Lacto-N-tetraose, or Lacto-N-neotetraose are not administered as part of the treatment.

In certain embodiments, one or more human milk oligosaccharides, e.g., synthetic human milk oligosaccharides, are administered to treat or prevent hyperammonemia. In some embodiments, the human milk oligosaccharides are or include one or more of 2′-fucosyllactose, 3-fucosyllactose, Lacto-N-tetraose, or Lacto-N-neotetraose. In particular embodiments, the human milk oligosaccharides are or include two or more of 2′-fucosyllactose, 3-fucosyllactose, Lacto-N-tetraose, or Lacto-N-neotetraose. In some embodiments, the human milk oligosaccharides are or includes 2′-fucosyllactose. In certain embodiments, the human milk oligosaccharides are or includes 3-fucosyllactose. In particular embodiments, the human milk oligosaccharides are or include Lacto-N-tetraose. In certain embodiments, the human milk oligosaccharides are or include Lacto-N-neotetraose. In certain embodiments, the human milk oligosaccharides are (i) 2′-fucosyllactose and 3-fucosyllactose, (ii) 2′-fucosyllactose and Lacto-N-tetraose, (iii) 2′-fucosyllactose and Lacto-N-neotetraose, (iv) 3-fucosyllactose and Lacto-N-tetraose, (v) 3-fucosyllactose and Lacto-N-neotetraose, or (vi) Lacto-N-tetraose and Lacto-N-neotetraose. In particular embodiments, the human milk oligosaccharides are (i) 3-fucosyllactose, Lacto-N-tetraose, and Lacto-N-neotetraose, (ii) 2′-fucosyllactose, Lacto-N-tetraose, and Lacto-N-neotetraose, (iii) 2′-fucosyllactose, 3-fucosyllactose, and Lacto-N-neotetraose, or (iv) 2′-fucosyllactose, 3-fucosyllactose, and Lacto-N-tetraose. In some embodiments, human milk oligosaccharides are 2′-fucosyllactose, 3-fucosyllactose, Lacto-N-tetraose, and Lacto-N-neotetraose.

In some embodiments, the percentage by weight of the administered human milk oligosaccharides comprising nitrogen is less than 50%. In certain embodiments, the percentage by weight of human milk oligosaccharides comprising nitrogen is less than 40%, 30%, 25%, 20%, 10%, 5%, or 1% of the human milk oligosaccharides that are administered.

In some embodiments, the subject in need thereof has, is at risk of having, or is suspected of having hyperammonemia. In certain embodiments, the subject in need thereof has, is suspected of having, or is at risk of having hepatic encephalopathy. In particular embodiments, the subject in need thereof has, is suspected of having, or is at risk of having a urea cycle disorder. In some embodiments, the subject in need thereof has or is suspected of having autism spectrum disorder.

### IV. FORMULATIONS

In certain embodiments, the provided at least one probiotic bacteria, e.g., B. longum subsp. infantis, and the provided prebiotics, e.g., the concentrated human milk permeate composition and/or the one or more synthetic oligosaccharides, are formulated together or separately, e.g., for administering to a human subject. In certain embodiments, the provided probiotic strain and prebiotics are formulated into the same pharmaceutical or nutritional composition. In particular embodiments, the provided probiotic strain and prebiotics are formulated into the different pharmaceutical or nutritional compositions.

The compositions, e.g., one or both of the prebiotics and the probiotic strains, described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the compositions described herein are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The probiotic strain and prebiotics described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, immediate-release, pulsatile-release, delayed-release, or sustained release). Suitable dosage amounts for the provided at least one probiotic bacteria strain may range from about 10⁵ to 10¹² bacteria, e.g., at, at about, or at least 10⁵ bacteria, 10⁶ bacteria, 10⁷ bacteria, 10⁸ bacteria, 10⁹ bacteria, 10¹⁰ bacteria, 10¹¹ bacteria, or 10¹² bacteria.

In some embodiments, the provided prebiotics, e.g., the concentrated human milk permeate composition and the one or more synthetic oligosaccharides, is administered to the subject generally in the range of about 20 mg to about 20 g, e.g., total prebiotic weight (such as weight of total HMO) per dose. In certain embodiments, the dose of the prebiotics, e.g., oligosaccharides, is from 50 mg and 50 g, 1 g to 20 g, 500 mg to 5 g, 2 g to 5 g, 5 g to 10 g, 8 g to 10 g, or is or is about 2 g, 4.5 g, 8 g, or 18 g. In some embodiments, a dose of the prebiotics is administered at least once per month, once per week, once every other day, or once per day, or twice per day. In some embodiments, a dose of the prebiotics is administered at least once, twice, three times, four times, five times, six times, eight times, ten times, or twelve times daily.

In some embodiments, the pharmaceutical compositions, e.g., pharmaceutical compositions containing one or both of the at least one probiotic bacteria strain or prebiotics, may be administered once or more daily, weekly, or monthly. The at least one probiotic bacteria strain and the prebiotics may be formulated, together or separately, into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the probiotic strain may be formulated in a solution of sodium bicarbonate, e.g., 1 molar solution of sodium bicarbonate (to buffer an acidic cellular environment, such as the stomach, for example).

In some embodiments, the pharmaceutical compositions containing the provided prebiotics and probiotic bacteria strain, e.g., together or as separate compositions, may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, methyl cellulose, hydroxypropylmethyl-cellulose, sodium carbomethylcellulose; and/or physiologically acceptable polymers such as) polyethylene glycol (PEG). Disintegrating agents may also be added, such as cross-linked agar, alginic acid or a salt thereof such as sodium alginate.

In certain embodiments, the concentrated human milk permeate composition is administered daily for at least 2, 3, 4, 5, 7, 10, 14, 21, or 28 days, e.g., consecutive days. In certain embodiments, the concentrated human milk permeate composition is administered in an amount of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day, e.g., by total weight of the human milk oligosaccharides of the composition. In particular embodiments, the concentrated human milk permeate composition is administered in an amount of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g total human milk oligosaccharides per day. In some embodiments, the concentrated human milk permeate composition is administered in an amount of between 0.1 g and 50 g; 0.5 g and 25 g, 1 g and 20 g, 2 g and 18 g, 1 g and 5 g, 2 g and 3 g, 3 g and 6 g, 4 g and 5 g, 5 g and 10 g, 8 g and 10 g, 10 g and 20 g, 15 g and 20 g, or 17 g and 19 g total human milk oligosaccharides per day. In some embodiments, the concentrated human milk permeate composition is administered in an amount of, of about, or of at least 2 g, 4.5 g, 6 g, 9 g, 12 g, 16 g, or 18 g total human milk oligosaccharides per day.

In certain embodiments, the one or more synthetic oligosaccharides, e.g., synthetic human milk oligosaccharides, are administered daily for at least 2, 3, 4, 5, 7, 10, 14, 21, or 28 days, e.g., consecutive days. In certain embodiments, the one or more synthetic oligosaccharides are administered in an amount of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day. In particular embodiments, the one or more synthetic oligosaccharides is administered in an amount of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day. In some embodiments, the one or more synthetic oligosaccharides are administered in an amount of between 0.1 g and 50 g; 0.5 g and 25 g, 1 g and 20 g, 2 g and 18 g, 1 g and 5 g, 2 g and 3 g, 3 g and 6 g, 4 g and 5 g, 5 g and 10 g, 8 g and 10 g, 10 g and 20 g, 15 g and 20 g, or 17 g and 19 g per day. In some embodiments, the one or more synthetic oligosaccharides are administered in an amount of, of about, or of at least 2 g, 4.5 g, 6 g, 9 g, 12 g, 16 g, or 18 g per day.

In particular embodiments, the probiotic strain is administered daily for at least 2, 3, 4, 5, 7, 10, 14, 21, or 28 days, e.g., consecutive days. In some embodiments, the at least one probiotic strain is administered in an amount of at least 1×10¹, 1×10², 1×10³, 1×10⁴, 1×10⁵, 1×10⁶, 5×10⁶, 1×10⁷, 1×10⁷, 5×10⁷, 1×10⁸, or 5×10⁸ colony forming units (CFU) per day. In various embodiments, the at least one probiotic strain is administered in an amount of at least 1×10¹, 1×10², 1×10³, 1×10⁴, 1×10⁵, 1×10⁶, 5×10⁶, 1×10⁷, 1×10⁷, 5×10⁷, 1×10⁸, or 5×10⁸ colony forming units (CFU) per dose or per day. In certain embodiments, the at least one probiotic strain is administered in an amount of between 1×10⁶ and 1×10¹², 5×10⁶ and 1×10¹⁰, 1×10⁷ and 1×10⁹, or 1×10⁷ and 1×10⁸ CFU per dose or per day. In some embodiments, the at least one probiotic strain is administered in an amount of, of about, or at least 5×10⁶ colony forming units (CFU) per dose or per day. In some embodiments, the at least one probiotic strain is administered in an amount of, of about, or at least 8×10⁷ colony forming units (CFU) per dose or per day.

Tablets or capsules can be prepared by conventional means with pharmaceutically acceptable excipients such as binding agents (e.g., pregelatinized maize starch, hydroxypropyl methylcellulose, carboxymethyl cellulose, polyethylene glycol, sucrose, glucose, sorbitol, starch, gum, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, such as with membrane selected from, but not limited to, polylactide, polyglycolic acid, polyanhydride, other biodegradable polymers, hydroymethylacrylate-methyl methacrylate (HEMA-MMA), multilayered HEMA-MMA-MAA, polyethylene glycol/poly pentamethylcyclopentasiloxane/polydimethylsiloxane (PEG/PD5/PDMS), siliceous encapsulates, cellulose acetate phthalate, calcium alginate, k-carrageenan-locust bean gum gel beads, poly(lactide-co-glycolides), carrageenan, starch polyanhydrides, starch polymethacrylates, and enteric coating polymers.

In some embodiments, the one or both of the prebiotics and the probiotic strain are enterically coated, such as in order to remain viable during transit through the stomach, reduce contact with bile acids in the small intestine, or for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In certain embodiments, the pharmaceutical compositions are formulated as liquid preparations. Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the bacteria described herein.

In some embodiments, the one or both of the probiotic strain and the prebiotics may be formulated in a composition suitable for administration to pediatric subjects. As is well known in the art, children differ from adults in many aspects, including different rates of gastric emptying, pH, gastrointestinal permeability, etc. Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, taste blockers. or suitable to be mixed in a foodstuff, e.g., applesauce. In one embodiment, a composition suitable for administration to pediatric subjects may also be suitable for administration to adults.

In certain embodiments, the pharmaceutical composition, e.g., containing one or both of the probiotic strain and the prebiotics, that is suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In some embodiments, the pharmaceutical composition, e.g., composition suitable for administration to pediatric subjects, may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, cherry, and chocolate.

In particular embodiments, the prebiotics and the probiotic strain may, together or separately, be orally administered, such as with an inert diluent or an assimilable edible carrier. In some aspects, the prebiotics and the probiotic strain may also be enclosed in a hard or soft-shell gelatin capsule, a hydroxypropylmethyl cellulose (HPMC) capsule, compressed into tablets, or incorporated directly into the subject's diet. For oral therapeutic administration, the probiotic strain and the prebiotics may, together or separately, be incorporated with excipients and used in the form of ingestible tablets, buccal tablets, troches, capsules, elixirs, suspensions, syrups, wafers, and the like. In some aspects, it may be necessary to coat or co-administer the pharmaceutical composition with a material to prevent inactivation of the probiotic strain and/or the prebiotics.

In some embodiments, the composition containing one or both of the probiotic strain and the prebiotics may be a nutritional or a comestible product, e.g., a food product or nutritional composition. In some embodiments, the composition is a nutritional composition such as food product. In certain embodiments, the food product or nutritional composition is or includes milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In some embodiments, the nutritional composition or food product is a fermented food, such as a fermented dairy product. In particular embodiments, the fermented dairy product is yogurt. In certain embodiments, the fermented dairy product is cheese, milk, cream, ice cream, milk shake, or kefir. In some embodiments, the probiotic strain of the invention, e.g., a B. longum subsp. infantis strain, is combined in a preparation containing other live bacterial cells intended to serve as probiotics. In some embodiments, the food product is a beverage. In one embodiment, the beverage is a fruit juice-based beverage or a beverage containing plant or herbal extracts. In certain embodiments, the food product or nutritional composition is a jelly or a pudding. Other food products suitable for administration of the probiotic strain and prebiotics provided herein are known, such as those described in U.S. Application Nos. 2015/0359894 and 2015/0238545. In yet another embodiment, the pharmaceutical composition of the invention is injected into, sprayed onto, or sprinkled onto a food product, such as bread, yogurt, or cheese.

In some embodiments, the composition, e.g., pharmaceutical composition, that includes one or both of the prebiotics and the probiotic strain is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

In some embodiments, disclosed herein are pharmaceutically acceptable compositions continuing one or both of the probiotic strain and prebiotics in single dosage forms. Single dosage forms may be in a liquid or a solid form. Single dosage forms may be administered directly to a subject without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition containing one or both of the probiotic strain and prebiotics may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a subject.

In certain embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see, e.g., U.S. Pat. No. 5,989,463). Examples of polymers used in sustained release formulations include, but are not limited to, poly((2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens of one or both of the prebiotics or the probiotic strain may be adjusted to provide a therapeutic response, e.g., to improve or maintain SCFA or lactate production. Dosing can depend on several factors, including severity and responsiveness of the disease, route of administration, time course of treatment (days to months to years), and time to amelioration of the disease. For example, a single bolus of one or both of the prebiotics and the probiotic strain may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

In some embodiments, ingredients (e.g., one or more of probiotic strain, concentrated human milk permeate composition, or one or more synthetic oligosaccharides along with pharmaceutically acceptable excipients) are supplied either separately or mixed together in unit dosage form, for example, as a dry lyophilized powder or water-free concentrate in a hermetically sealed container such as an ampoule or sachet indicating the quantity of active agent.

The pharmaceutical compositions, e.g., containing one or both of the prebiotics and probiotic strain, may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include polydextrose, dextrins (e.g., maltodextrin (e.g., a native maltodextrin or a resistant maltodextrin)), inulin, β-glucan, resistant starches (e.g., resistant maltodextrin), hydrocolloids (e.g., one or more of gum Arabic, pectin, guar gum, alginate, carrageenan, xanthan gum and cellulose gum), corn syrup solids and the like and polysorbate 80. Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the pharmaceutical compositions, e.g., containing one or both of the probiotic strain and the prebiotics are administered with food. In alternate embodiments, the pharmaceutical composition is administered before or after eating food. The pharmaceutical compositions may be administered in combination with one or more dietary modifications, e.g., low-protein diet and amino acid supplementation. The dosage of the pharmaceutical compositions and the frequency of administration may be selected based on the severity of the symptoms and the progression of the disorder. The appropriate therapeutically effective dose and/or frequency of administration can be selected by a treating clinician.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the described or claimed subject matter. This applies regardless of the breadth of the range.

Throughout this disclosure, ranges that are presented or expressed as “between” two endpoints, e.g., “between A and B” are understood to include the endpoints, e.g. “A” and “B”, unless otherwise indicated.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” a value means within a range of 25%, ±10%, ±5%, ±1%, ±0.1%, or 0.01% of the value.

As used herein the term “pharmaceutical composition” means, for example, a mixture or formulation containing a specified amount, e.g., a therapeutically effective amount, of an active ingredient such as a human milk fraction, in a pharmaceutically acceptable carrier to be administered to a mammal, e.g., a human.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response, and other problem complications commensurate with a reasonable benefit/risk ratio. Such reasonable benefit/risk ratios may be determined by of skill as a matter of routine.

By “human milk oligosaccharide(s)” (also referred to herein as “HMO(s)”) is meant a family of structurally diverse unconjugated glycans that are found in human breast milk. As used herein human milk oligosaccharides include oligosaccharides found in human milk that contain lactose at the reducing end and, typically, fucose, sialic acid or N-acetylglucosamine at the non-reducing end (Morrow et al., J. Nutri. 2005 135:1304-1307). Unless otherwise indicated, human milk oligosaccharides also encompass 3′-sialyllactose (3′-SL) and 6′-sialyllactose (6′-SL) oligosaccharides that are found in human milk.

Unless otherwise noted, a number of human milk oligosaccharides, e.g., “at least 5 human milk oligosaccharides,” refers to the number of unique species of human milk oligosaccharides, e.g., human milk oligosaccharides having different chemical structures or formulas.

Glycans in milk are found as oligosaccharides or conjugated to milk proteins as glycoproteins, or lipid as glycolipids etc. HMO are free glycans that constitute the third most abundant component of human milk, after lactose and lipid (Morrow, 2005). The majority of HMO, however, are not metabolized by the infant and can be found in infant feces largely intact.

By “consisting essentially” of, as used herein refers to compositions containing particular recited components while excluding other major bioactive factors.

“Probiotic” as used herein, refers to any live, non-pathogenic microorganisms, e.g., bacteria, which can confer health benefits to a host organism, e.g., a mammal such as a human, that contains an appropriate amount of the microorganism. In some aspects, those of skill in the art may readily identify species, strains, and/or subtypes of non-pathogenic bacteria that are recognized as probiotic bacteria. Examples of probiotic bacteria may include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006).

“Bifidobacterium” or “Bifidobacteria” as used herein, refers to a genus of gram-positive, nonmotile, anaerobic bacteria. In some aspects, Bifidobacterium are ubiquitous inhabitants of the gastrointestinal tract, vagina, and mouth of mammals, including humans. In certain aspects, Bifidobacteria are one of the major genera of bacteria that make up the gastrointestinal tract microbiota in mammals. In certain aspects, some or all species, subspecies, or strains of Bifidobacterium are probiotics.

The term “dysbiosis” as used herein refers to a state of the microbiota of the gut or other body area in a subject, in which the normal diversity and/or function of the microbial populations is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health. According to non-limitative examples, essential functions may include enhancement of the gut mucosal barrier, direct or indirect reduction and elimination of invading pathogens, enhancement of the absorption of specific substances, and suppression of GI inflammation.

As used herein, the terms “gut microbiome” and “intestinal microbiome” are used interchangeably unless otherwise noted.

The term “essentially” such as when used in the phrase “essentially all” of a given substance may be used to infer that the substance, e.g., oligosaccharides, includes unavoidable impurities, e.g., no more impurities than what might be unavoidable with standard techniques for manufacture, formulation, transporting, and storage. Likewise, when used in the phrase “essentially free” of a given substance (or “essentially no” or “essentially none of” a given substance) may mean no more of the given substance than is unavoidable, e.g., as an impurity.

The term “internalization” such as in reference to an internalization of an oligosaccharide by a bacterial cell refers to the transfer of the oligosaccharide from the outside of the bacterial cell to the inside of the bacterial cell. Unless otherwise indicated, “internalization of an oligosaccharide” refers to the internalization of the intact oligosaccharide.

Unless otherwise indicated, the term “synthetic human milk oligosaccharide” or “synthetic oligosaccharide” refers to an oligosaccharide that is not collected, purified, extracted, isolated, or otherwise obtained from human milk. Synthetic human milk oligosaccharides may include human milk oligosaccharides that are chemically synthesized and/or synthesized by methods that include fermentation of carbohydrates with genetically modified microorganisms.

### VI. EXAMPLES

The following examples are included for illustrative purposes only and are not intended to limit the scope of the invention.

### Example 1: Manufacture of a Concentrated Human Milk Permeate Composition

Human milk from previously screened and approved donors was tested to verify donor identity and then mixed together to generate a pooled bulk of donor milk. In a clean room environment, the pool of donor milk was further tested, including to confirm the absence of specific pathogens and bovine proteins. After testing, the pooled donor milk was filtered through a 200 μm filter, heated to a temperature of at least 63° C. for 30 minutes, and then cooled to between 22° C. and 26° C. The pooled human milk was then transferred to a centrifuge to separate the cream from the skim. The resulting skim milk was processed through an ultra-filtration system with a 10 kDa membrane, and the material that was passed through the filter was collected as the permeate fraction. The permeate was frozen and stored at approximately −20° C. Each permeate lot was tested to confirm quality parameters, including a minimal HMO concentration of approximately 0.2 to 0.4 g/L total HMOs, prior to its release for further processing.

Multiple qualified lots of the permeate were thawed and pooled. The pH of the pooled permeate was adjusted to a target pH of 4.5±0.2. The permeate was then heated to approximately 50° C. Lactase enzyme was added to the permeate at a 0.1% w/w concentration and incubated at approximately 50° C. for 60 minutes. The permeate and lactase enzyme mixture then was cooled to between 20° C. and 30° C. and clarified by depth filtration (Filtrox CH113P). The resulting depth filter filtrate was processed through an ultra-filtration skid (Biomax-10K membrane) to remove the lactase. The ultra-filtered permeate was then concentrated by nanofiltration using membranes with an estimated 400 to 500 Dalton molecular weight cut-off (GE G-5 UF). The concentrated HMO composition was then pasteurized and clarified though 0.2 μm sterile filters. This final HMO composition was then filled into containers and stored at ≤−20° C. The final concentrations of HMO were targeted to between 84.5 to 105.4 g/L and quantified using high performance anion exchange chromatography with pulsed amperometry detection (HPAEC-PAD) with commercially available standards.

### Example 2: Administration of B. longum Subsp. infantis and a Concentrated Human Milk Permeate Composition to Healthy Adult Subjects

Healthy adult men and women between the ages of 18 and 44 were enrolled as subjects in a study to evaluate administration of a B. longum subsp. infantis probiotic and a concentrated human milk permeate composition prepared as described in Example 1.

The subjects were separated into cohorts that were assigned to receive some or all of the B. longum subsp. infantis probiotic, the concentrated human milk permeate composition, and an over-the-counter acid reducing drug. Subjects that received the B. longum subsp. infantis probiotic consumed a dose of at least 8×10⁹ Colony forming units (CFU) daily for the first seven days (days 1-7) of the clinical study. Subjects assigned to receive the concentrated human milk permeate composition consumed two doses daily for the first fourteen days of the study (days 1-14) for total daily doses of 4.5 g/day, 9 g/day, or 18 g/day HMO. Subjects of an additional cohort were assigned to receive an acid reducing drug and the B. longum subsp. infantis probiotic on days 1-7 and 18 g/day HMO on days 1-14 and then a second treatment cycle of the same beginning on day 29 of the study. A summary of the initial study design is shown in Table E1.

TABLE E1
Experimental Cohorts

| | Target HMO Amount | | B. infantis | Acid Reducer |
|---|---|---|---|---|
| Cohort | (Days 1-14) | | (Day 1-7) | (Day 1-7) |
| 1 | 0 | g/day | Yes | Yes |
| 2 | 18 | g/day | No | Yes |
| 3 | 4.5 | g/day | Yes | Yes |
| 4 | 9 | g/day | Yes | Yes |
| 5* | 18 | g/day | Yes | Yes |
| 6 | 18 | g/day | Yes | No |

*Cohort 5 undergoes the same dosing regimen of HMO, B. infantis, and PPI twice. A first dosing regimen from days 1-14 and a second dosing regimen from days 29-43.

The cohorts assigned to receive an acid reducing drug were initially assigned to receive the proton pump inhibitor omeprazole with sodium bicarbonate (ZEGERID™) 1-2 hours prior to consuming the probiotic. After the participation of the first 12 subjects, the study continued with the following changes: subjects assigned to receive doses of B. longum subsp. infantis and the concentrated human milk permeate composition at doses of 4.5 and 9 g/day HMO were not administered an acid reducing drug, and subjects assigned to Cohort 5 were further split into two sub-cohorts, Cohort 5A, which received the H2-receptor antagonist famotidine on days 29-36, and Cohort 5B, which received no acid reducing drugs on days 29-36. One subject from Cohort 5 who had received Omeprazole on days 29-36 was included in Cohort 5A. A summary of the experimental cohorts is summarized in Table E2.

TABLE E2
Summary of Experimental Cohorts

| Cohort | Subjects | B. infantis | HMO | | Acid Reducing Drug |
|---|---|---|---|---|---|
| 1 | 10 | Yes | 0 | g | 2/10 Omeprazole, 8/10 none |
| 2 | 10 | No | 18 | g | 2/10 Omeprazole, 8/10 none |
| 3 | 10 | Yes | 4.5 | g | 2/10 Omeprazole, 8/10 none |
| 4 | 10 | Yes | 9 | g | 2/10 Omeprazole, 8/10 none |
| 5A | 1 | Yes | 18 | g | Omeprazole/Omeprazole |
| 5A | 4 | Yes | 18 | g | Omeprazole/Famotidine |
| 5B | 5 | Yes | 18 | g | Omeprazole/None |
| 6 | 9 | Yes | 18 | g | None |

Stool samples were collected from the subjects at day 1 prior to administration of the B. longum subsp. infantis and/or the concentrated HMO mixture, and at days 5, 8, 15, 22, and 29 of the study. Stool samples from subjects in Cohorts 5A and 5B were also collected on days 33, 36, 43, 50, and 57. Aliquots of stool were refrigerated immediately after collection and then frozen at about −70° C. or colder within 24 hours. DNA was extracted from the stool aliquots and analyzed with species- and strain-specific quantitative PCR analysis to evaluate B. longum subsp. infantis colonization. The quantitative PCR was performed similar to as described in Lawley et al., PeerJ. 2017 May 25; 5:e3375 with forward and reverse primers identical to SEQ ID NOS: 54 and 55 and a probe sequence identical to SEQ ID NO: 56.

For Cohort 1 (administered B. longum subsp. infantis but not the concentrated HMO mixture), B. longum subsp. infantis was detected in the stool from all ten subjects on days 5 and 8. B. longum subsp. infantis was not detected in stool collected from these subjects on day 1 (prior to the administration of B. longum subsp. infantis) or on days 15, 22, and 29 (after the administration of B. longum subsp. infantis), with the exception of detectable levels of B. longum subsp. infantis in stool collected from a single Cohort 1 subject on day 15.

For Cohort 2 (administered the concentrated HMO mixture but not B. longum subsp. infantis), B. longum subsp. infantis was not detected in stools from any of the ten subjects collected at days 1, 8, 15, 22 and 29. Among the stools collected from Cohort 2 at day 5, B. longum subsp. infantis was only detected in the stool from a single subject. A follow-up analysis suggested that this B. longum subsp. infantis detection may have been a false positive due to a technical error. As only one stool sample collected from only one individual at a single time point had detectable levels of B. longum subsp. infantis, these data are consistent with reported absence of B. infantis in the adult gastrointestinal tract (Underwood et al., Pediatr Res. 2015; 77(1-2):229-235).

The qPCR results from samples collected from subjects in Cohorts 3, 4, 5, and 6 (administered B. longum subsp. infantis and the concentrated human milk permeate composition at 4.5 g, 9 g, and 18 g of HMO per day, respectively) were assessed to identify subjects with successful B. longum subsp. infantis colonization or engraftment. Positive qPCR results on days 5, 8, and 15 were required for a subject to be considered successfully engrafted or colonized with B. longum subsp. infantis. Results are summarized in Table E3. Data from only 9 subjects were evaluated from Cohort 6 as one subject in the cohort withdrew consent after the baseline timepoint.

TABLE E3
Subjects colonized with B. infantis

| | Treatment | | Engraftment |
|---|---|---|---|
| Cohort | B. inf | HMO | (Colonized subjects/total) |
| 3 | Yes | 4.5 g/day | 3/10 |
| 4 | Yes | 9 g/day | 2/10 |
| 5A and | Yes | 18 g/day | 5/10 |
| 5B | | | |
| 6 | Yes | 18 g/day | 6/9 |

As discussed above, subjects of Cohorts 5A and 5B received treatment with acid reducing drugs (Omeprazole on days 1-7) in addition to B. longum subsp. infantis and 18 g per day of HMO. As shown in Table E3, combined results from Cohorts 5A and 5B on days 5, 8, and 15 were similar to the results observed from Cohort 6 (not administered acid reducing drugs), consistent with no discernable effect of administration of acid reducing drugs on B. longum subsp. infantis engraftment.

Cohorts 5A and 5B also received a second round of treatments with the concentrated HMO mixture and B. longum subsp. infantis after a two-week washout period. Of the five subjects in Cohorts 5A and 5B deemed to have successful engraftment of B. longum subsp. infantis at days 5, 8, and 15, two subjects sustained successful engraftment again in the second round of dosing (detectable B. longum subsp. infantis in samples collected at days 33, 36, 43). These data are consistent with an ability for subsequent colonization of B. longum subsp. infantis to occur after prior treatments with B. longum subsp. infantis and HMO.

Taken together, these data are consistent with an ability of B. longum subsp. infantis to engraft in the human adult intestinal microbiome when administered with the concentrated human milk permeate composition. These data are also consistent with a maintenance of B. longum subsp. infantis engraftment with continued administration of the human milk permeate composition.

### Example 3: Synbiotic Administration of B. longum Subsp. infantis and Human Milk Oligosaccharides to Healthy Adult Subjects

Healthy adult men and women are enrolled as subjects in a study investigating synbiotic administration of a B. longum subsp. infantis probiotic and human milk oligosaccharides. Subjects are assigned to treatment groups that are administered one or more of: an initial treatment with antibiotics (e.g., vancomycin and/or metronidazole), a B. longum subsp. infantis probiotic, a concentrated human milk permeate (e.g., as described in Example 1), synthetic human milk oligosaccharides (e.g., a mixture of synthetic 2′-FL and LNnT).

Exemplary treatment groups are shown in Table E4. The study may contain treatment groups where subjects receive an initial antibiotic treatment along with doses of the B. longum subsp. infantis probiotic. The probiotic may be administered in conjunction with a prebiotic, such as the concentrated human milk permeate or one or more synthetic human milk oligosaccharides (e.g., a mixture of synthetic 2′-FL and LNnT). Treatment with the prebiotics will persist for two weeks after the final dose of the B. longum subsp. infantis probiotic has been administered.

TABLE E4
Exemplary Treatment Groups

| Exemplary | | B. longum | Concentrated | |
|---|---|---|---|---|
| Dosing | | subsp. | Human Milk | |
| Group | Antibiotics | infantis | Permeate | Synthetic HMO |
| 1 | Day 1-5 | None | None | None |
| 2 | Day 1-5 | Day 1-14 | Day 1-28 | None |
| 3 | Day 1-5 | Day 1-14 | None | None |
| 4 | Day 1-5 | Day 1-14 | Day 1-9 | Day 10-28 |

Stool samples are collected from the subjects prior to administration of the B. longum subsp. infantis and/or the antibiotics and at various timepoints during and after the treatments. Aliquots of stool are refrigerated immediately after collection and then frozen. DNA will be extracted from the stool and analyzed with species- and strain-specific quantitative PCR analysis to evaluate B. longum subsp. infantis colonization. The quantitative PCR is performed similar to as described Example 2. Quantitation limits are demonstrated by qualification assays demonstrating the lower limit of detection in order to establish minimum log-fold change in B. longum subsp. infantis.

Results may indicate that B. longum subsp. infantis is detectable in subjects that receive treatment with the B. longum subsp. infantis probiotic. In subjects that also receive doses of concentrated human milk permeate, results may indicate that colonization of B. longum subsp. infantis persists for the duration of the treatment with the human milk permeate. Colonization of B. longum subsp. infantis may also persist for the duration of treatment with the synthetic human milk oligosaccharides. Such an observation is consistent with successful maintenance of B. longum subsp. infantis colonization by administration of synthetic human milk oligosaccharides.

### Example 4: In Vitro Growth of B. longum Subsp. infantis

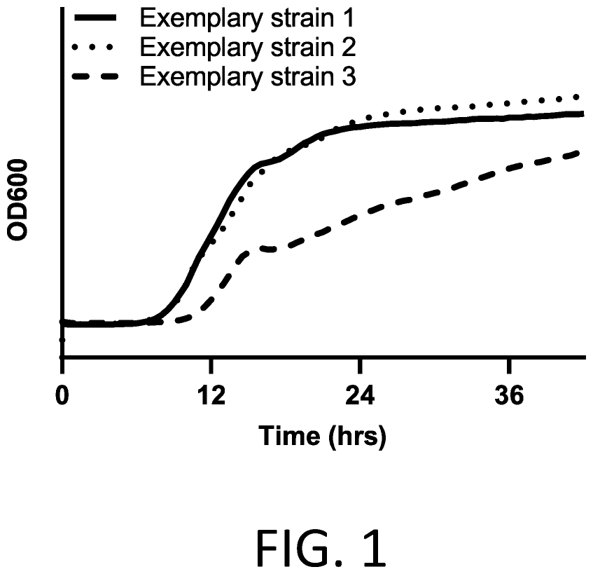

In vitro growth of B. longum subsp. infantis with synthetic human milk oligosaccharides (HMOs) as the sole carbon source was assessed. B. longum subsp. infantis was incubated with synthetically-derived 2′-fucosyllactose (2′-FL) and lacto-N-neotetraose (LNnT). The growth of the B. longum subsp. infantis was assessed by measuring the optical density at 600 nm (OD600) with an automated spectrophotometer at regular 30-minute intervals. As shown in FIG. 1, growth of B. longum subsp. infantis in the presence of 2′FL and LNnT was observed. Results are consistent with an ability of B. longum subsp. infantis to utilize synthetically derived HMO as a carbon source.

1-60. (canceled)

61. A method for maintaining engraftment of a probiotic strain of B. longum subsp. infantis in a subject in need thereof, the method comprising administering to the subject one or more synthetic human milk oligosaccharides, wherein the subject is an adult who has previously been administered B. longum subsp. infantis and a concentrated human milk permeate composition comprising human milk oligosaccharides, thereby maintaining the engraftment of the probiotic strain of B. longum subsp. infantis.

62. The method of claim 61, wherein the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose.

63. The method of claim 61, wherein the one or more synthetic human milk oligosaccharides are administered at least once every other day for at least 3, 5, 7, 10, 14, 21, or 28 days in an amount of at least 10 g, 15 g, 18 g, 20 g, or 25 g of total human milk oligosaccharides per day.

64. The method of claim 63, wherein the one or more synthetic human milk oligosaccharides are administered in an amount of from about 10 g to about 25 g of total human milk oligosaccharides per day.

65. The method of claim 61, wherein the B. longum subsp. infantis was previously administered to the subject in an amount of at least 1×108 colony forming units (CFU) per day for at least 3 days, 7 days, 9 days, or 14 days.

66. The method of claim 61, wherein the concentrated human milk permeate composition comprises at least 10, at least 25, at least 50, or at least 100 human milk oligosaccharides, and wherein the concentrated human milk permeate composition comprises 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose.

67. The method of claim 61, wherein the concentrated human milk permeate composition was obtained from human milk permeate resulting from ultrafiltration of human skim milk, wherein the human skim milk is obtained by removing cream from pooled human milk, and wherein the pooled human milk is pooled from the milk of at least 50, 100, or 150 human milk donors.

68. The method of claim 61, wherein the concentrated human milk permeate composition was previously administered in an amount of at least 10 g, 15 g, 18 g, 20 g, or 25 g of total human milk oligosaccharides per day for at least 3 days.

69. The method of claim 61, wherein the concentrated human milk permeate composition was previously administered in an amount from about 10 g to about 25 g of total human milk oligosaccharides for at least 7, 9, or 14 days.

70. The method of claim 61, wherein the B. longum subsp. infantis and the concentrated human milk permeate composition were previously administered to the subject on the same day for at least 3 days, 5 days, 7 days, 9 days, or 14 days.

71. The method of claim 61, wherein the method comprises administering the one or more synthetic human milk oligosaccharides in an amount of from about 10 g to about 25 g of total human milk oligosaccharides per day for at least 7 days, wherein the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose;
wherein the B. longum subsp. infantis was administered in an amount of at least 1×108 colony forming units (CFU) per day for at least 7 days;
wherein the concentrated human milk permeate composition was administered in an amount of at least 10 g of total human milk oligosaccharides per day for at least 7 days; and
wherein the B. longum subsp. infantis and the concentrated human milk permeate composition were previously administered to the subject on the same day for at least 3 days, 5 days, or 7 days.

72. A method for treating or preventing a disease, disorder, or condition in a subject in need thereof, the method comprising administering to the subject one or more synthetic human milk oligosaccharides, wherein the subject is an adult who has previously been administered B. longum subsp. infantis and a concentrated human milk permeate composition comprising human milk oligosaccharides, thereby treating or preventing the disease, disorder, or condition; wherein the disease, disorder, or condition is associated with one or more of dysbiosis of the intestinal microbiome, inflammation, infection, allergy, or immune dysfunction.

73. The method of claim 72, wherein the method comprises administering the one or more synthetic human milk oligosaccharides in an amount of from about 10 g to about 25 g of total human milk oligosaccharides per day for at least 7 days, wherein the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose;
wherein the B. longum subsp. infantis was administered in an amount of at least 1×108 colony forming units (CFU) per day for at least 7 days;
wherein the concentrated human milk permeate composition was administered in an amount of at least 10 g of total human milk oligosaccharides per day for at least 7 days; and
wherein the B. longum subsp. infantis and the concentrated human milk permeate composition were previously administered to the subject on the same day for at least 3 days, 5 days, or 7 days.

74. The method of claim 72, wherein the disease, disorder, or condition comprises one or more of obesity, type II diabetes, a chronic inflammatory disease, an autoimmune disease, an infection, an infectious disease domination, bowel resection, or a condition associated with chronic diarrhea.

75. The method of claim 72, wherein the disease, disorder, or condition comprises one or more of irritable bowel syndrome (IBS), inflammatory bowel disease (IBD), short bowel syndrome (SBS), celiac disease, small intestinal bacterial overgrowth (SIBO), gastroenteritis, leaky gut syndrome, pouchitis, or gastric lymphoma.

76. The method of claim 72, wherein the disease, condition, or disorder is associated with an infection, optionally wherein the infection comprises a bacterial infection or gut domination.

77. A method for treating or preventing graft versus host disease in a subject in need thereof, the method comprising administering to the subject one or more synthetic human milk oligosaccharides, wherein the subject is an adult who has received or will receive an allogenic hematopoietic stem cell transplant, and wherein the subject has previously been administered B. longum subsp. infantis and a concentrated human milk permeate composition comprising human milk oligosaccharides, thereby treating or preventing the graft versus host disease.

78. The method of claim 77, wherein the subject receives an allogenic hematopoietic stem cell transplant, and wherein the colonization phase takes place beginning at least 7, 14, or 21 days prior to the allogenic hematopoietic stem cell transplant and lasts at least until 7, 14, 21, 28, 35 days after the allogenic hematopoietic stem cell transplant.

79. The method of claim 77, wherein the subject receives treatment with antibiotics beginning at least 5 days prior to the allogenic hematopoietic stem cell transplant lasting until at least 5 days after the allogenic hematopoietic stem cell transplant, optionally wherein the antibiotics comprises one or more of a fourth-generation cephalosporins, a glycopeptide, a piperacillin-tazobactam, a carbapenem, an aminoglycoside, or a quinolone; and wherein the colonization phase lasts until at least 10 days after the end of the treatment with antibiotics.

80. The method of claim 77, wherein the method comprises administering the one or more synthetic human milk oligosaccharides in an amount of from about 10 g to about 25 g of total human milk oligosaccharides per day for at least 7 days, wherein the one or more synthetic human milk oligosaccharides comprise one or more of 2′-fucosyllactose, 3-fucosyllactose, 3′-sialyllactose, 6′-sialyllactose, lacto-N-tetraose, lacto-N-neotetraose, or difucosyllactose;
wherein the B. longum subsp. infantis was administered in an amount of at least 1×10⁸ colony forming units (CFU) per day for at least 7 days;
wherein the concentrated human milk permeate composition was administered in an amount of at least 10 g of total human milk oligosaccharides per day for at least 7 days; and
wherein the B. longum subsp. infantis and the concentrated human milk permeate composition were previously administered to the subject on the same day for at least 3 days, 5 days, or 7 days.
